SEQUENCE LISTING (73 sequences)

SEQ ID NO: 1 - protein, 328 aa, Homo sapiens
  SITE 1..328: note = UniProtKB - Q6ZMC9 (SIG15_HUMAN)
MEKSIWLLACLAWVLPTGSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTVALLLGALGFKALLLLGVLAARAARRRPEHLDTPDTPPRSQAQESNYENLSQMNPRSPPATMCSP

SEQ ID NO: 2 - protein, 342 aa, Mus musculus
  SITE 1..342: note = UniProtKB - A7E1W8 (A7E1W8_MOUSE)
MEGSLQLLACLACVLQMGSLVKTRRDASGDLLNTEAHSAPAQRWSMQVPAEVNAEAGDAAVLPCTFTHPHRHYDGPLTAIWRSGEPYAGPQVFRCTAAPGSELCQTALSLHGRFRLLGNPRRNDLSLRVERLALADSGRYFCRVEFTGDAHDRYESRHGVRLRVTAAAPRIVNISVLPGPAHAFRALCTAEGEPPPALAWSGPAPGNSSAALQGQGHGYQVTAELPALTRDGRYTCTAANSLGRAEASVYLFRFHGAPGTSTLALLLGALGLKALLLLGILGARATRRRLDHLVPQDTPPRSQAQESNYENLSQMSPPGHQLPRVCCEELLSHHHLVIHHEK

SEQ ID NO: 3 - protein, 107 aa, synthetic construct
  REGION 1..107: note = Synthetic: 1H3 Light Chain Variable Region Amino Acid Sequence
DIQMTQASSSLSVSLGGRVTITCKASDHINNWLAWYQQKPGNAPRLLISGATSLETGVPSRFSGSGSGKDYTLSITSLQTEDVATYYCQQYWSSPLTFGAGTKLELK

SEQ ID NO: 4 - protein, 11 aa, synthetic construct
  REGION 1..11: note = Synthetic: 1H3 Light Chain CDR1
KASDHINNWLA

SEQ ID NO: 5 - protein, 7 aa, synthetic construct
  REGION 1..7: note = Synthetic: 1H3 hVL1 CDR2
GATSLET

SEQ ID NO: 6 - protein, 9 aa, synthetic construct
  REGION 1..9: note = Synthetic: 1H3 Light Chain CDR3
QQYWSSPLT

SEQ ID NO: 7 - DNA, 321 bp, synthetic construct
  misc_feature 1..321: note = Synthetic: Nucleic acid sequence encoding the 1H3 light chain variable region
  source 1..321: mol_type = other DNA
gacatccagatgacacaggcttcatcctccttgtctgtatctctaggaggcagagtcaccattacttgcaaggcaagtgaccacattaataattggttggcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagttctcctctcacgttcggtgctgggaccaagctggagctgaaa

SEQ ID NO: 8 - protein, 107 aa, synthetic construct
  REGION 1..107: note = Synthetic: humanized anti-SIGLEC-15 antibody 1H3 hVL1, variable light chain amino acid
DIQMTQSPSSLSASVGDRVTITCKASDHINNWLAWYQQKPGKAPKLLISGATSLETGVPSRFSGSGSGTDYTFTISSLQPEDIATYYCQQYWSSPLTFGGGTKVEIK

SEQ ID NO: 9 - protein, 107 aa, synthetic construct
  REGION 1..107: note = Synthetic: hVL2 antibody variable light chain amino acid sequence
DIQMTQSPSSLSASVGDRVTITCKASDHINNWLAWYQQKPGKVPKLLISGATSLETGVPSRFSGSGSGTDYTLTISSLQPEDVATYYCQQYWSSPLTFGGGTKVEIK

SEQ ID NO: 10 - protein, 107 aa, synthetic construct
  REGION 1..107: note = Synthetic: humanized anti-SIGLEC-15 1H3 hVL3 antibody variable light chain amino acid sequence
DIQMTQSPSSLSASVGDRVTITCKASDHINNWLAWYQQKPGKAPKLLISGATSLETGVPSRFSGSGSGTDYTLTISSLQPEDVATYYCQQYWSSPLTFGGGTKVEIK

SEQ ID NO: 11 - protein, 123 aa, synthetic construct
  REGION 1..123: note = Synthetic: 1H3 Heavy Chain Variable Region Amino Acid Sequence
QVQLKESGPGLVAPSQSLSITCTVSGFSLSNYGVHWVRQPPGKGLEWLVLIWSDGSTTYNSALKSRLSISKDNSKSQVFLKMNSLQTGDTAMYYCARHPYDDYSGYYYTMDYWGQGTSVTVSS

SEQ ID NO: 12 - protein, 5 aa, synthetic construct
  REGION 1..5: note = Synthetic: 1H3 Heavy Chain CDR1
NYGVH

SEQ ID NO: 13 - protein, 16 aa, synthetic construct
  REGION 1..16: note = Synthetic: 1H3 hVH1 CDR2
LIWSDGSTTYNSALKS

SEQ ID NO: 14 - protein, 15 aa, synthetic construct
  REGION 1..15: note = Synthetic: 1H3 Heavy Chain CDR3
HPYDDYSGYYYTMDY

SEQ ID NO: 15 - DNA, 369 bp, synthetic construct
  misc_feature 1..369: note = Synthetic: nucleic acid sequence encoding the 1H3 heavy chain variable region
  source 1..369: mol_type = other DNA
caggtgcagctgaaggagtcaggacctggcctggtggcgccctcacagagcctgtccatcacatgcaccgtctcagggttctcattaagcaattatggtgtacactgggttcgccagcctccaggaaagggtctggagtggctggtactgatatggagtgatggaagcacaacctataattcagctctcaaatccagactgagcatcagcaaggacaactccaagagccaagttttcttaaaaatgaacagtctccaaactggtgacacagccatgtactactgtgccagacatccctatgatgattattccggctattactatactatggactactggggtcaaggaacctcagtcaccgtctcctca

SEQ ID NO: 16 - protein, 123 aa, synthetic construct
  REGION 1..123: note = Synthetic: humanized anti-SIGLEC 15 antibody 1H3 hVH1 variable heavy chain amino acid sequence
QVQLQESGPGLVKPSETLSLTCTVSGFSLSNYGVHWVRQPPGKGLEWIVLIWSDGSTTYNSALKSRVTISKDTSKNQVSLKLSSVTAADTAVYYCARHPYDDYSGYYYTMDYWGQGTLVTVSS

SEQ ID NO: 17 - protein, 122 aa, synthetic construct
  REGION 1..122: note = Synthetic: humanized anti-SIGLEC 15 1H3 hVH2 antibody variable heavy chain amino acid sequence
QVQLQESGPGLVKPSETLSLTCTVSGFSLSNYGVHWVRQPPGKGLEWIGLIWSDGSTTYASALKSRVTISKDTSKNQVSLKLSSVTAADTAVYYCARHPYDDYSGYYYTMDYWGQGTLVTVS

SEQ ID NO: 18 - protein, 16 aa, synthetic construct
  REGION 1..16: note = Synthetic: 1H3 hVH2 CDR2
LIWSDGSTTYASALKS

SEQ ID NO: 19 - protein, 122 aa, synthetic construct
  REGION 1..122: note = Synthetic: humanized anti-SIGLEC 15 1H3 hVH3 antibody variable heavy chain amino acid sequence
QVQLQESGPGLVKPSETLSLTCTVSGFSLSNYGVHWVRQPPGKGLEWIGLIWSDGSTTYNPSLKSRVTISKDTSKNQVSLKLSSVTAADTAVYYCARHPYDDYSGYYYTMDYWGQGTLVTVS

SEQ ID NO: 20 - protein, 16 aa, synthetic construct
  REGION 1..16: note = Synthetic: 1H3 hVH3 CDR2
LIWSDGSTTYNPSLKS

SEQ ID NO: 21 - protein, 122 aa, synthetic construct
  REGION 1..122: note = Synthetic: humanized anti-SIGLEC 15 antibody 1H3 hVH4 variable heavy chain amino acid sequence
QVQLQESGPGLVKPSETLSLTCTVSGFSLSNYGVHWVRQPPGKGLEWIGLIWSEGSTTYASALKSRVTISKDTSKNQVSLKLSSVTAADTAVYYCARHPYDDYSGYYYTMDYWGQGTLVTVS

SEQ ID NO: 22 - protein, 16 aa, synthetic construct
  REGION 1..16: note = Synthetic: 1H3 hVH4 CDR2
LIWSEGSTTYASALKS

SEQ ID NO: 23 - protein, 108 aa, synthetic construct
  REGION 1..108: note = Synthetic: 5G12 Light Chain Variable Region Amino Acid Sequence
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIKR

SEQ ID NO: 24 - protein, 11 aa, synthetic construct
  REGION 1..11: note = Synthetic: 5G12 Light Chain CDR1
KASQDINSYLS

SEQ ID NO: 25 - protein, 7 aa, synthetic construct
  REGION 1..7: note = Synthetic: CDR2 of 5G12 hVL
RANRLVD

SEQ ID NO: 26 - protein, 9 aa, synthetic construct
  REGION 1..9: note = Synthetic: CDR3 of 5G12 hVL
LQYDEFPYT

SEQ ID NO: 27 - DNA, 321 bp, synthetic construct
  misc_feature 1..321: note = Synthetic: nucleic acid sequence encoding the 5G12 light chain variable region
  source 1..321: mol_type = other DNA
gacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaagctggaaataaaa

SEQ ID NO: 28 - protein, 107 aa, synthetic construct
  REGION 1..107: note = Synthetic: humanized anti-SIGLEC-15 antibody 5G12 hVL1 variable light chain amino acid sequence
DIQMTQSPSSLSASVGDRVTITCKASQDINSYLSWFQQKPGKAPKTLIYRANRLVDGVPSRFSGSGSGTDYTLTISSLQPEDFATYYCLQYDEFPYTFGGGTKVEIK

SEQ ID NO: 29 - protein, 107 aa, synthetic construct
  REGION 1..107: note = Synthetic: humanized anti-SIGLEC-15 antibody 5G12 hVL2 variable light chain amino acid sequence
DIQMTQSPSSLSASVGDRVTITCKASQDINTYLSWFQQKPGKAPKTLIYRANRLVDGVPSRFSGSGSGTDYTLTISSLQPEDFATYYCLQYDEFPYTFGGGTKVEIK

SEQ ID NO: 30 - protein, 11 aa, synthetic construct
  REGION 1..11: note = Synthetic: CDR1 of 5G12 hVL2
KASQDINTYLS

SEQ ID NO: 31 - protein, 107 aa, synthetic construct
  REGION 1..107: note = Synthetic: humanized anti-SIGLEC-15 antibody 5G12 hVL3 variable light chain amino acid sequence
DIQMTQSPSSLSASVGDRVTITCKASQDINVYLSWFQQKPGKAPKTLIYRANRLVDGVPSRFSGSGSGTDYTLTISSLQPEDFATYYCLQYDEFPYTFGGGTKVEIK

SEQ ID NO: 32 - protein, 11 aa, synthetic construct
  REGION 1..11: note = Synthetic: CDR1 of 5G12 hVL
KASQDINVYLS

SEQ ID NO: 33 - protein, 107 aa, synthetic construct
  REGION 1..107: note = Synthetic: humanized anti-SIGLEC-15 antibody 5G12 hVL4 variable light chain amino acid sequence
DIQMTQSPSSLSASVGDRVTITCKASQDIQSYLSWFQQKPGKAPKTLIYRANRLVDGVPSRFSGSGSGTDYTLTISSLQPEDFATYYCLQYDEFPYTFGGGTKVEIK

SEQ ID NO: 34 - protein, 11 aa, synthetic construct
  REGION 1..11: note = Synthetic: CDR1 of 5G12 hVL4
KASQDIQSYLS

SEQ ID NO: 35 - protein, 107 aa, synthetic construct
  REGION 1..107: note = Synthetic: humanized anti-SIGLEC-15 antibody 5G12 hVL5 variable light chain amino acid sequence
DIQMTQSPSSLSASVGDRVTITCKASQDINVYLSWFQQKPGKAPKTLIYRANRLTSGVPSRFSGSGSGTDYTLTISSLQPEDFATYYCLQYDEFPYTFGGGTKVEIK

SEQ ID NO: 36 - protein, 7 aa, synthetic construct
  REGION 1..7: note = Synthetic: CDR2 of 5G12 hVL5
RANRLTS

SEQ ID NO: 37 - protein, 122 aa, synthetic construct
  REGION 1..122: note = Synthetic: 5G12 Heavy Chain Variable Region Amino Acid Sequence
QVQLQQPGAELVKPGASVKMSCKASGYTFTSYWITWVIQRPGQGLEWIGDIYCGSDTMHYNEKFKNKATLTVDTSSSTAYMQLSSLTSEDSAVYYCARWWDYGSSYDYFDYWGQGTTLTVSS

SEQ ID NO: 38 - protein, 10 aa, synthetic construct
  REGION 1..10: note = Synthetic: 5G12 Heavy Chain CDR1
GYTFTSYWIT

SEQ ID NO: 39 - protein, 17 aa, synthetic construct
  REGION 1..17: note = Synthetic: 5G12 Heavy Chain CDR2
DIYCGSDTMHYNEKFKN

SEQ ID NO: 40 - protein, 13 aa, synthetic construct
  REGION 1..13: note = Synthetic: 5G12 Heavy Chain CDR3
WWDYGSSYDYFDY

SEQ ID NO: 41 - DNA, 366 bp, synthetic construct
  misc_feature 1..366: note = Synthetic: nucleic acid sequence encoding the 5G12 heavy chain variable region
  source 1..366: mol_type = other DNA
caggtccaactgcagcagcctggggctgagcttgtgaagcctggggcttcagtgaagatgtcctgcaaggcttctggctacaccttcaccagctactggataacctgggtgatacagaggccgggacaaggccttgagtggattggagatatttattgtggtagtgatactatgcactacaatgagaagttcaagaacaaggccacactgactgtagacacatcctccagcacagcctacatgcagctcagcagcctgacatctgaggactctgcggtctattactgtgcaagatggtgggactacggtagtagctacgactactttgactactggggccaaggcaccactctcacagtctcctca

SEQ ID NO: 42 - protein, 122 aa, synthetic construct
  REGION 1..122: note = Synthetic: 5G12 hVH1variable heavy chain amino acid sequence
QVQLVQSGAEVKKPGASVKVSCKASGYTFTSYWITWVRQAPGQGLEWMGDIYSGSDTMHYAEKFQGRVTLTVDTSTSTAYMELSSLRSEDTAVYYCARWWDYGSSYDYFDYWGQGTLVTVSS

SEQ ID NO: 43 - protein, 5 aa, synthetic construct
  REGION 1..5: note = Synthetic: CDR1 of 5G12 hVH
SYWIT

SEQ ID NO: 44 - protein, 17 aa, synthetic construct
  REGION 1..17: note = Synthetic: CDR2 of 5G12 hVH
DIYSGSDTMHYAEKFQG

SEQ ID NO: 45 - protein, 122 aa, synthetic construct
  REGION 1..122: note = Synthetic: 5G12 hVH2 variable heavy chain amino acid sequence
QVQLVQSGAEVKKPGASVKVSCKASGYTFTSYWITWVRQAPGQGLEWMGDIYSGSDTTHYAEKFQGRVTLTVDTSTSTAYMELSSLRSEDTAVYYCARWWDYGSSYDYFDYWGQGTLVTVSS

SEQ ID NO: 46 - protein, 122 aa, synthetic construct
  REGION 1..122: note = Synthetic: 5G12 hVH3 variable heavy chain amino acid sequence
QVQLVQSGAEVKKPGASVKVSCKASGYTFTSYWISWVRQAPGQGLEWMGDIYSGSDTTHYAEKFQGRVTLTVDTSTSTAYMELSSLRSEDTAVYYCARWWDYGSSYDYFDYWGQGTLVTVSS

SEQ ID NO: 47 - protein, 5 aa, synthetic construct
  REGION 1..5: note = Synthetic: CDR1 of 5G12 hVH3
SYWIS

SEQ ID NO: 48 - protein, 640 aa, Homo sapiens
  SITE 1..640: note = UniProtKB - Q9HCJ2 LRC4C_HUMAN
MLNKMTLHPQQIMIGPRFNRALFDPLLVVLLALQLLVVAGLVRAQTCPSVCSCSNQFSKVICVRKNLREVPDGISTNTRLLNLHENQIQIIKVNSFKHLRHLEILQLSRNHIRTIEIGAFNGLANLNTLELFDNRLTTIPNGAFVYLSKLKELWLRNNPIESIPSYAFNRIPSLRRLDLGELKRLSYISEGAFEGLSNLRYLNLAMCNLREIPNLTPLIKLDELDLSGNHLSAIRPGSFQGLMHLQKLWMIQSQIQVIERNAFDNLQSLVEINLAHNNLTLLPHDLFTPLHHLERIHLHHNPWNCNCDILWLSWWIKDMAPSNTACCARCNTPPNLKGRYIGELDQNYFTCYAPVIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTKIIIGCFVAITLMAAVMLVIFYKMRKQHHRQNHHAPTRTVEIINVDDEITGDTPMESHLPMPAIEHEHLNHYNSYKSPFNHTTTVNTINSIHSSVHEPLLIRMNSKDNVQETQI

SEQ ID NO: 49 - protein, 486 aa, synthetic construct
  REGION 1..486: note = Synthetic: exemplary fusion protein is Siglec15 ECD-IgG1 Fc Fusion Protein (L234F/L235E/P331S)
MEWSWVFLFFLSVTTGVHSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 467 aa, synthetic construct
  REGION 1..467: note = Synthetic: fusion protein
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 496 aa, synthetic construct
  REGION 1..496: note = Synthetic: Siglec-15 - FES (hIgG1 FES mutation silenced Fc receptor binding)
MEWSWVFLFFLSVTTGVHSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGDKTHTCPPCPDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 477 aa, synthetic construct
  REGION 1..477: note = Synthetic: Siglec-15 Fc (IgG1 Fc; FES)
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGDKTHTCPPCPDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 488 aa, synthetic construct
  REGION 1..488: note = Synthetic: Siglec-15 Fc (IgG4 Fc; G4P)
MEWSWVFLFFLSVTTGVHSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 54 - protein, 469 aa, synthetic construct
  REGION 1..469: note = Synthetic: Siglec-15 Fc (IgG4 Fc; G4P)
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - protein, 214 aa, synthetic construct
  REGION 1..214: note = Synthetic: 1H3 light chain variable domain-IgG1 Fc fusion protein
DIQMTQSPSSLSASVGDRVTITCKASDHINNWLAWYQQKPGKAPKLLISGATSLETGVPSRFSGSGSGTDYTFTISSLQPEDIATYYCQQYWSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 214 aa, synthetic construct; identical to SEQ ID NO: 55
  REGION 1..214: note = Synthetic: humanized anti-1H3 hVL1-kappa constant domain having an amino acid sequence

SEQ ID NO: 57 - protein, 214 aa, synthetic construct
  REGION 1..214: note = Synthetic: humanized anti-1H3 hVL2-kappa constant domain antibody
DIQMTQSPSSLSASVGDRVTITCKASDHINNWLAWYQQKPGKVPKLLISGATSLETGVPSRFSGSGSGTDYTLTISSLQPEDVATYYCQQYWSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 214 aa, synthetic construct
  REGION 1..214: note = Synthetic: humanized anti-1H3 hVL3-kappa constant domain antibody
DIQMTQSPSSLSASVGDRVTITCKASDHINNWLAWYQQKPGKAPKLLISGATSLETGVPSRFSGSGSGTDYTLTISSLQPEDVATYYCQQYWSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 349 aa, synthetic construct
  REGION 1..349: note = Synthetic: 1H3 heavy chain variable domain-IgG1 Fc fusion protein
QVQLKESGPGLVAPSQSLSITCTVSGFSLSNYGVHWVRQPPGKGLEWLVLIWSDGSTTYNSALKSRLSISKDNSKSQVFLKMNSLQTGDTAMYYCARHPYDDYSGYYYTMDYWGQGTSVTVSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 359 aa, synthetic construct
  REGION 1..359: note = Synthetic: 1H3 heavy chain variable domain Fc (IgG1 Fc; FES)
QVQLKESGPGLVAPSQSLSITCTVSGFSLSNYGVHWVRQPPGKGLEWLVLIWSDGSTTYNSALKSRLSISKDNSKSQVFLKMNSLQTGDTAMYYCARHPYDDYSGYYYTMDYWGQGTSVTVSSDKTHTCPPCPDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 351 aa, synthetic construct
  REGION 1..351: note = Synthetic: 1H3 LC-Fc (IgG4 Fc; G4P)
QVQLKESGPGLVAPSQSLSITCTVSGFSLSNYGVHWVRQPPGKGLEWLVLIWSDGSTTYNSALKSRLSISKDNSKSQVFLKMNSLQTGDTAMYYCARHPYDDYSGYYYTMDYWGQGTSVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 62 - protein, 349 aa, synthetic construct
  REGION 1..349: note = Synthetic: 1H3 hVH1 heavy chain variable domain-IgG1 Fc fusion protein
QVQLQESGPGLVKPSETLSLTCTVSGFSLSNYGVHWVRQPPGKGLEWIVLIWSDGSTTYNSALKSRVTISKDTSKNQVSLKLSSVTAADTAVYYCARHPYDDYSGYYYTMDYWGQGTLVTVSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 359 aa, synthetic construct
  REGION 1..359: note = Synthetic: 1H3 hVH1 heavy chain variable domain Fc (IgG1 Fc; FES)
QVQLQESGPGLVKPSETLSLTCTVSGFSLSNYGVHWVRQPPGKGLEWIVLIWSDGSTTYNSALKSRVTISKDTSKNQVSLKLSSVTAADTAVYYCARHPYDDYSGYYYTMDYWGQGTLVTVSSDKTHTCPPCPDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 351 aa, synthetic construct
  REGION 1..351: note = Synthetic: 1H3 hVH1-Fc (IgG4 Fc; G4P)
QVQLQESGPGLVKPSETLSLTCTVSGFSLSNYGVHWVRQPPGKGLEWIVLIWSDGSTTYNSALKSRVTISKDTSKNQVSLKLSSVTAADTAVYYCARHPYDDYSGYYYTMDYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 348 aa, synthetic construct
  REGION 1..348: note = Synthetic: humanized 1H3 hVH2 heavy chain variable domain-IgG1 Fc fusion protein
QVQLQESGPGLVKPSETLSLTCTVSGFSLSNYGVHWVRQPPGKGLEWIGLIWSDGSTTYASALKSRVTISKDTSKNQVSLKLSSVTAADTAVYYCARHPYDDYSGYYYTMDYWGQGTLVTVSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 358 aa, synthetic construct
  REGION 1..358: note = Synthetic: 1H3 hVH2 heavy chain variable domain Fc (IgG1 Fc; FES)
QVQLQESGPGLVKPSETLSLTCTVSGFSLSNYGVHWVRQPPGKGLEWIGLIWSDGSTTYASALKSRVTISKDTSKNQVSLKLSSVTAADTAVYYCARHPYDDYSGYYYTMDYWGQGTLVTVSDKTHTCPPCPDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 350 aa, synthetic construct
  REGION 1..350: note = Synthetic: 1H3 hVH2-Fc (IgG4 Fc; G4P)
QVQLQESGPGLVKPSETLSLTCTVSGFSLSNYGVHWVRQPPGKGLEWIGLIWSDGSTTYASALKSRVTISKDTSKNQVSLKLSSVTAADTAVYYCARHPYDDYSGYYYTMDYWGQGTLVTVSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 68 - protein, 348 aa, synthetic construct
  REGION 1..348: note = Synthetic: humanized 1H3 hVH3 heavy chain variable domain-IgG1 Fc fusion protein
QVQLQESGPGLVKPSETLSLTCTVSGFSLSNYGVHWVRQPPGKGLEWIGLIWSDGSTTYNPSLKSRVTISKDTSKNQVSLKLSSVTAADTAVYYCARHPYDDYSGYYYTMDYWGQGTLVTVSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 358 aa, synthetic construct
  REGION 1..358: note = Synthetic: 1H3 hVH3 heavy chain variable domain Fc (IgG1 Fc; FES)
QVQLQESGPGLVKPSETLSLTCTVSGFSLSNYGVHWVRQPPGKGLEWIGLIWSDGSTTYNPSLKSRVTISKDTSKNQVSLKLSSVTAADTAVYYCARHPYDDYSGYYYTMDYWGQGTLVTVSDKTHTCPPCPDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 350 aa, synthetic construct
  REGION 1..350: note = Synthetic: 1H3 hVH3-Fc (IgG4 Fc; G4P)
QVQLQESGPGLVKPSETLSLTCTVSGFSLSNYGVHWVRQPPGKGLEWIGLIWSDGSTTYNPSLKSRVTISKDTSKNQVSLKLSSVTAADTAVYYCARHPYDDYSGYYYTMDYWGQGTLVTVSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 71 - protein, 348 aa, synthetic construct
  REGION 1..348: note = Synthetic: 1H3 hVH4 heavy chain variable domain-IgG1 Fc fusion protein
QVQLQESGPGLVKPSETLSLTCTVSGFSLSNYGVHWVRQPPGKGLEWIGLIWSEGSTTYASALKSRVTISKDTSKNQVSLKLSSVTAADTAVYYCARHPYDDYSGYYYTMDYWGQGTLVTVSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 358 aa, synthetic construct
  REGION 1..358: note = Synthetic: 1H3 hVH4 heavy chain variable domain Fc (IgG1 Fc; FES)
QVQLQESGPGLVKPSETLSLTCTVSGFSLSNYGVHWVRQPPGKGLEWIGLIWSEGSTTYASALKSRVTISKDTSKNQVSLKLSSVTAADTAVYYCARHPYDDYSGYYYTMDYWGQGTLVTVSDKTHTCPPCPDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 350 aa, synthetic construct
  REGION 1..350: note = Synthetic: 1H3 hVH4-Fc (IgG4 Fc; G4P)
QVQLQESGPGLVKPSETLSLTCTVSGFSLSNYGVHWVRQPPGKGLEWIGLIWSEGSTTYASALKSRVTISKDTSKNQVSLKLSSVTAADTAVYYCARHPYDDYSGYYYTMDYWGQGTLVTVSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2024/0400678 A1
(43) Pub. Date: Dec. 5, 2024

(54) ANTIBODIES FOR SIGLEC-15 AND METHODS OF USE THEREOF TO TREAT DISUSE OSTEOPOROSIS

(71) Applicant: NEXTCURE, INC., Beltsville, MD (US)

(72) Inventors: Priyanka KOTHARI, Beltsville, MD (US); Weiping QIN, Beltsville, MD (US); Solomon LANGERMANN, Beltsville, MD (US)

(73) Assignee: NEXTCURE, INC., Beltsville, MD (US)

(21) Appl. No.: 18/671,264

(22) Filed: May 22, 2024

Related U.S. Application Data
(60) Provisional application No. 63/503,618, filed on May 22, 2023.

(51) Int. Cl. C07K 16/28; A61K 38/00; A61K 39/00; A61K 45/06; A61P 19/10; C07K 14/705

(52) U.S. Cl. CPC C07K 16/2803; A61K 45/06; A61P 19/10; C07K 14/70503; A61K 38/00; A61K 2039/505; C07K 2317/24; C07K 2317/565; C07K 2317/76; C07K 2319/30

(57) ABSTRACT

Provided herein are compositions that immunospecifically bind to Siglec-15, small molecules, peptides, or other entities that may bind Siglec-15 in a similar fashion. Methods of use are provided for Siglec-15 targeting therapeutics to restore bone homeostasis by inhibiting bone resorption and increase bone formation, specifically in non-ambulatory people or in people with disuse osteoporosis. The therapeutics referenced here may be used to overcome the need for mechanical loading on the bone to maintain high quality skeleton.

### REFERENCE TO RELATED APPLICATIONS

This application claims benefit to and priority to U.S. Provisional Patent Application Ser. No. 63/503,618, filed on May 22, 2023, which is hereby incorporated by this reference in its entirety.

### STATEMENT REGARDING FEDERALLY SPONSORED RESEARCH

This invention was made with government support under 101 RX002089-01A2 awarded by the Department of Veterans Affairs. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on May 22, 2024, is named “064467.116US.xml” and is 107,936 bytes in size. The sequence listing contained in this .XML file is part of the specification and is hereby incorporated by reference herein in its entirety.

### FIELD

The invention is generally related to the field of immunomodulation, specifically a novel approach to mitigate osteoporosis due to immobilization or non-ambulatory state.

### BACKGROUND

Of the total non-institutionalized population in the US in 1988, 3.8% (8.8 million people) were estimated not to be able to perform any major activity (LP, Interview Survey, 1988). Immobilization osteoporosis represents a wide spectrum of conditions and disorders. A few representative examples encompass a range of rates of bone loss including individuals confined to bed rest (e.g., stroke, and poliomyelitis) at 0.1% per week, microgravity at 0.25% per week, and spinal cord injury (SCI) at 1% per week during the initial months (Vico et al, Lancet, 355:9215 (2000); Szollar et al, Am J Phys Med Rehabil, 77(1):28-35 (1998); Garland et al, J Spinal Cord Med, 27(3):202-6 (2004); Warden et al, Osteoporos Int, 13(7):586-92 (2002); Leblanc et al, J Bone Miner Res, 5(8):843-50 (1990)). In contrast, post-menopausal osteoporosis, a non-immobilizing condition associated with bone loss that has heightened awareness in the medical community, has a bone loss of 3-5% per year when not prescribed antiresorptive medications (Recker et al, J Bone Miner Res, 15(10):1965-73 (2000)). Although efficacious strategies have been developed for other forms of osteoporosis (e.g. sex-hormone deficient, glucocorticoid-induced, nutritional deficiency osteoporosis, etc.), the ability to maintain bone when load is acutely reduced is, at present, far from clinically satisfactory (Bauman and Cardozo, Osteoporosis, Osteoporosis Fourth Edition: Academic Press (2013)).

In the United States, there are approximately 288,000 people with SCI, and more than 42,000 of those with SCI are Veterans; about 17,700 new cases of SCI occur annually (National Spinal Cord Injury Statistical Center, 2018; Vestergaard et al, Spinal Cord, 36(11):790-6 (1998)). VA provides care to more than 27,000 Veterans with SCI and related disorders each year, making the department the largest health care system in the world providing lifelong spinal cord care (US Dept Veteran Affairs, 2022).

Over the first couple of years after SCI, 50-60% of BMD (bone mineral density) may be lost at the epiphyseal and metaphyseal regions of the long bones of the lower extremities (Cirnigliaro et al, J Bone Miner Res Plus, 4(8):e10375 (2020)). Almost immediately after SCI, bone resorption and calcium excretion are greatly increased. Histologic measures of bone biopsy samples demonstrated that bone formation was reduced individuals with SCI (Minaire et al, Calcif Tiss Res, 17(1):57-73 (1974)). Because SCI causes rapid and extensive loss of sublesional bone, these individuals are predisposed to an increased risk of fracture (Bauman and Cardozo, Osteoporosis, 2013; Battaglino et al, Curr Osteo Rep, 10(4):278-85 (2012); Qin et al, Ann NY Acad Sci, 1211:66-84 (2010)). Bone may be lost in trabecular regions of the sublesional skeleton at a rate as great as ˜1%/week for the first year after SCI and continues to be lost at a rapid rate for the next year or so, while cortical bone is lost by endocortical resorption at an increased rate for at least the initial 7 years after paralysis (Zehnder et al, Osteoporos Int, 15(3):180-9 (2004); Bauman et al, Osteoporos Int, 10(2):123-7 (1999)). The rate at which bone is lost after SCI is over 10 times greater than that of postmenopausal osteoporosis (PMO) (3-5%/year) (Qin et al, Curr Osteoporos Rep, 8(4):212-8 (2010)) and is more severe than other types of disuse osteoporosis, such as microgravity (0.25%/week) (Vico et al, Lancet, (2000)) and prolonged bed rest (0.1%/week) (Leblanc et al, J Bone Miner Res, 1990). The prevalence of fractures is reported to be as high as 25 to 46 percent in persons with SCI (Vestergaard et al, Spinal Cord, 36(11):790-6 (1998)) with fractures occurring even with minor stress or trauma (Asselin et al, J Spinal Cord Med, 34(1):52-9 (2011)). The average hospital stay following fracture in a patient with SCI is 35 days, 7-times longer than those for admissions without fractures (Morse et al, PM & R, 1(3):240-4 (2009); Morse et al, Osteoporos Int, 20(3):385-92 (2009)). The prolonged hospital stay is, in part, because most of these patients experience local, general, or orthopedic complications. Importantly, a heightened risk of fracture due to bone loss may preclude participation in activity-based rehabilitation, the use of promising exoskeletal devices or modalities of spinal cord stimulation for ambulation, and may prevent the ability to participate in many treatments related to advances made in neurorepair. At the present time, there is no practical intervention available to safely restore a sufficient fraction of the bone loss to be of clinical relevance, highlighting the need for novel approaches and treatments. Thus, persons with SCI have a well appreciated need for an efficacious treatment approach to improve sublesional bone integrity, and in particular at the distal femur and proximal tibia around knee.

Therefore, this invention provides therapeutic compositions for the treatment of severe loss of bone that occurs in regions of the skeleton affected by any non-ambulatory or reduced ambulatory state, including paralysis after SCI.

### SUMMARY

In accordance with the purpose(s) of this invention, as embodied and broadly described herein, this invention, in one aspect, relates a method of treating bone loss in a subject in need thereof, comprising an effective amount of composition comprising an immunomodulatory agent that modulates Siglec-15 expression, ligand binding, crosslinking, Siglec-15 mediated signaling or a combination thereof selected from a group consisting of (i) a soluble Siglec-15 polypeptide or fusion protein, (ii) a function blocking or function activating anti-Siglec-15 antibody, (iii) a monoclonal antibody or antigen-binding fragment thereof that depletes Siglec-15 positive cells, and (iv) combinations thereof; to modulate Siglec-15 mediated signaling in subject in need thereof.. In one embodiment, the monoclonal antibody or antigen-binding fragment thereof comprises a light chain variable region (LCVR) having an amino acids sequence of at least 98%, 99% or more sequence identity to SEQ ID NO: 3, 7, 8, 9, 10, 23, 27, 28, 29, 31, 33, 35, or a variants thereof and a heavy chain variable region (HCVR) having an amino acid sequence of least 98%, 99% or more sequence identity to SEQ ID NO: 11, 15, 16, 17, 19, 21, 37, 41, 42, 45, 46 or variants thereof, and a heavy chain variable region (HCVR) having an amino acid sequence of least 98%, 99% or more sequence identity to SEQ ID NO: 11, 15, 16, 17, 19, 21, 37, 41, 42, 45, 46 or variants thereof, and wherein the monoclonal antibody or antigen-binding fragment thereof exhibits binding to Siglec-15. In another embodiment, the monoclonal antibody or antigen-binding fragment thereof binds specifically to three heavy chain CDRs (HCDR1, HCDR2 and HCDR3) contained within any one of the heavy chain variable region (HCVR) sequences having 95%, 96%, 97%, 98%, 99% or more sequence identity to sequences selected from the group consisting of SEQ ID NOs: 16, 17, 19, 21, 42, 45, and 46; and three light chain CDRs (LCDR1, LCDR2 and LCDR3) contained within any one of the light chain variable region (LCVR) sequences having 95%, 96%, 97%, 98%, 99% or more sequence identity to sequences selected from the group consisting of SEQ ID NOs: 8, 9, 10, 28, 29, 31, 33, and 35. The monoclonal antibody or antigen-binding fragment thereof may be comprised of one or more of the heavy chain CDR sequences having an amino acid sequences with 95%, 96%, 97%, 98%, 99% or more sequence identity to sequences selected from the group consisting of SEQ ID NO: 11, 15, 16, 17, 19, 21, 37, 41, 42, 45 and 46 and light chain CDR (LCDR) sequences having an amino acid sequences with 95%, 96%, 97%, 98%, 99% or 100% sequence identity to sequences selected from the group consisting of SEQ ID NO:4, 5, 6, 24, 25, 26, 30, 32, 34, and 36.

In one embodiment, the monoclonal antibody or antigen-binding fragment thereof comprises (a) an HCDR1 domain having 95%, 96%, 97%, 98%, 99% or more sequence identity to sequences selected from the group consisting of SEQ ID NOs: 12, 38, 43, and 47; (b) a HCDR2 domain having 95%, 96%, 97%, 98%, 99% or more sequence identity to sequences selected from the group consisting of SEQ ID NO: 13, 18, 20, 22, 39, and 44; (c) an HCDR3 domain having 95%, 96%, 97%, 98%, 99% or more sequence identity to sequences selected from the group consisting of SEQ ID NO: 14 and 40; (d) an LCDR1 domain having 95%, 96%, 97%, 98%, 99% or more sequence identity to sequences selected from the group consisting of SEQ ID NOs: 4, 24, 30, 32 and 34; (e) an LCDR2 domain having 95%, 96%, 97%, 98%, 99% or more sequence identity to sequences selected from the group consisting SEQ ID NOs: 5, 25, and 36; and (f) a LCDR3 domain having 95%, 96%, 97%, 98%, 99% or more sequence identity to sequences selected from the group consisting SEQ ID NO: 6, and 26.

In another embodiment, the monoclonal antibody or antigen-binding fragment thereof is administered with an additional therapeutic agent that functions to enhance bone formation or inhibit bone loss. The therapeutic agent may be selected from a group consisting of bisphosphonates, cytokines, other immunotherapeutics, enzymes, antibiotics, growth factors, growth inhibitors, hormones (including testosterone and parathyroid hormone), hormone antagonists, antibodies and bioactive fragments thereof (including humanized, single chain, and chimeric antibodies), antigen and vaccine formulations (including adjuvants), peptide drugs, anti-inflammatories, molecules that target additional bone metabolic pathways and modulate osteoclastogenesis or osteoblastogenesis. In another embodiment, the monoclonal antibody or antigen-binding fragment thereof binds osteoclasts to reduce osteoclast maturation and osteoclastogenesis while inducing osteoblastogenesis. Thus, the SIGLEC-15 monoclonal antibody is established as a unique therapeutic agent in bone disease including disuse osteoporosis in that it has antiresorptive activity while preserving bone formation function for a net anabolic outcome.

In another embodiment, the immunomodulatory agent is a SIGLEC-15 fusion protein comprises an extracellular domain of SIGLEC-15 for functional variant thereof linked to an immunoglobulin domain, wherein the fusion protein inhibits, reduces, or blocks SIGLEC-15 mediated signal transduction. In another embodiment, the SIGLEC-15 fusion protein comprises the amino acid sequence of any one of SEQ ID NO:1 or 2, or a functional variant thereof linked to an immunoglobulin domain, wherein the fusion modulates SIGLEC-15 mediated signal transduction. In another embodiment, the SIGLEC-15 fusion protein has 95%, 99%, or 100% to any one of SEQ ID NO: 49, 50, 51, 52, 53, and 54.

In another aspect, the invention relates to a method of promoting an immune response in a subject having bone loss comprising administering an effective amount of the monoclonal antibody of claim 1 in an amount effective to inhibit or reduce bone loss, maintain bone formation or promote rebuilding of the bone in the subject, wherein the subject has disuse osteoporosis due to immobilization, reduction of mechanical loading pressure and activity that is required for homeostatic bone maintenance or remodeling. The subject's immobilization, reduction of pressure and activity may be due to a condition selected from a group including spinal cord injury, hip fracture, cerebral palsy, stroke, paralysis, comas, time spent at zero gravity, medical conditions leading to prolonged bed rest, diseases or syndromes by which a person is dependent on a wheelchair for mobility purposes, including but not limited to Guillain-Barre syndrome, Ehlers-Danlos syndrome, Multiple Sclerosis, Muscular Dystrophy, Amyotrophic Lateral Sclerosis, Spina bifida, poliomyelitis (polio) or Parkinson's disease. Likewise, the application of SIGLEC-15 monoclonal antibody can also be extended to other conditions of osteoporosis with activity-limiting rheumatological diseases.

In one embodiment, promoting the immune response in the subject maintains bone formation while inhibiting bone resorption, inhibits loss of bone mineral density, inhibits declines in trabecular bone volume and connectivity by increasing trabecular number and preserving trabecular space, increases bone stiffness and preserves bone strength, reduces osteoclast maturation and osteoclastogenesis, and induces osteoblastogenesis.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIGS. 1A-1I show the binding of two anti-Siglec-15 antibodies (1H3 and 5G12) to human osteoclasts generated in vitro. In FIG. 1A-1H osteoclasts were generated in vitro from PBMCs by differentiating with MCSF and RANKL. Antibody staining was performed as described below. Antibody binding is shown in red (AF-647 conjugated antibodies and nuclei are shown in blue (Hoescht). Control isotype antibodies are shown for comparison. FIG. 1I shows the quantification of binding intensity. The experiment was completed with samples from two donors. Representative data from one donor shown herein.

FIG. 2A-2D show the effect of an anti-Siglec-15 antibody on increasing BMD without significantly affecting body weight. Areal BMD for the indicated sites determined by analysis of images acquired by DXA scanning. Data are expressed as mean±SEM. N=11-13/group. Significance of differences was determined using one-way ANOVA with a Newman-Keuls test post hoc. **P<0.01 and ***P<0.001.

FIGS. 3A-3I show the effect of an anti-Siglec-15 antibody on trabecular architecture of the distal femur as assessed by micro-CT. FIGS. 3A-3C are representative 3D images of trabecular microarchitecture. Measurements are shown for: FIG. 3D is a bar graph showing trabecular bone volume over total volume (BV/TV). FIG. 3E is a bar graph showing trabecular number (Tb.N, mm-1). FIG. 3F is a bar graph showing trabecular separation (Tb.Sp, μm). FIG. 3G is a bar graph showing connectivity density (Conn.D, mm-3). FIG. 3H is a bar graph showing structure model index (SMI, ranges from 0 to 3 with 0=platelike and 3=rodlike). FIG. 3I is a bar graph showing bone stiffness (N/mm) by finite element analysis, respectively. Data are expressed as mean±SEM. N=10/group. Significance of differences was determined using one-way ANOVA with a Newman-Keuls test post hoc. *P<0.05, **P<0.01 and ***P<0.001.

FIGS. 4A-4E show the effect of anti-Siglec-15 antibody on bone resorption of trabecular bone at the distal femur. FIGS. 4A-4C are representative sections of trabecular bone from the femur immunostained for TRAP (20×). The reddish areas of TRAP staining on trabecular surfaces represent osteoclasts. Parameters of trabecular bone resorption by histomorphometric quantification: FIG. 4D-4E are bar graphs showing (FIG. 4D) eroded surface/bone surface (ES/BS) and ((FIG. 4E)) osteoclast number/bone perimeter (N.Oc/B.Pm). Data are expressed as mean±SEM. N=6-7/group. Significance of differences was determined using one-way ANOVA with a Newman-Keuls test post hoc. *p<0.05.

FIGS. 5A-5F are images and representation of the effect of anti-Siglec-15 antibody on bone formation. FIGS. 5A-5C are representative 6-mm-thick bone specimen showing double-labeling of calcein green and xylenol orange under fluorescence microscopy (20×). Measurement is shown for (FIG. 5D) BFR/BS (mm3/mm2/year), (FIG. 5E) MS/BS (%), and (FIG. 5F) MAR (mm/day). Data are expressed as mean±SEM. N=5-7/group. NS, no significance. BFR/BS, bone formation rate; MS/BS, mineralizing surface/bone surface; MAR, mineral apposition rate.

FIGS. 6A-6G are representations of the effect of anti-Siglec-15 antibody on osteoclast maturation. FIGS. 6A-6C show TRAP staining of cultured osteoclasts. FIG. 6D is a bar graph showing the quantification of TRAP+ multinucleated cells. FIGS. 6E-6F are bar graphs showing the gene expression of bone resorption markers in cultured osteoclasts determined by qPCR: (FIG. 6E) TRAP, (FIG. 6F) Calcitonin (Calr). FIG. 6G shows Osteoblastic regulator: miR-183 levels determined by quantitative PCR. N=4-5/group. Significance of differences was determined using one-way ANOVA with a Newman-Keuls test post hoc. *p<0.05, **p<0.01, ***p<0.001.

FIGS. 7A-7J show the effects of anti-Siglec-15 antibody on osteoblastic differentiation. FIGS. 7A-7F are representative images and quantification of ALP staining (CFU-F (FIGS. 7A-7C)) and von Kossa staining (CFU-ob (FIGS. 7D-7F)) of marrow stromal cells. FIGS. 7G-7I are bar graphs showing the mRNA levels of bone formation markers by qPCR: osteocalcin and BSP. Data are expressed as mean±SEM. N=4/group. Significance of differences was determined using one-way ANOVA with a Newman-Keuls test post hoc. **p<0.01, ***p<0.001.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

As used herein, a molecule is said to be able to “immunospecifically bind” a second molecule if such binding exhibits the specificity and affinity of an antibody to its cognate antigen. Antibodies are said to be capable of immunospecifically binding to a target region or conformation (“epitope”) of an antigen if such binding involves the antigen recognition site of the immunoglobulin molecule. An antibody that immunospecifically binds to a particular antigen may bind to other antigens with lower affinity if the other antigen has some sequence or conformational similarity that is recognized by the antigen recognition site as determined by, e.g., immunoassays, BIACORE® assays, or other assays known in the art, but would not bind to a totally unrelated antigen. Preferably, however, antibodies (and their antigen binding fragments) will not cross-react with other antigens. Antibodies may also bind to other molecules in a way that is not immunospecific, such as to FcR receptors, by virtue of binding domains in other regions/domains of the molecule that do not involve the antigen recognition site, such as the Fc region.

As used herein, a molecule is said to “physiospecifically bind” a second molecule if such binding exhibits the specificity and affinity of a receptor to its cognate binding ligand. A molecule can be capable of physiospecifically binding to more than one other molecule.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

As used herein the term “modulate” relates to a capacity to alter an effect, result, or activity (e.g, signal transduction). Such modulation can agonistic or antagonistic. Antagonistic modulation can be partial (i.e., attenuating, but not abolishing) or it can completely abolish such activity (e.g., neutralizing). Modulation can include internalization of a receptor following binding of an antibody or a reduction in expression of a receptor on the target cell. Agonistic modulation can enhance or otherwise increase or enhance an activity (e.g., signal transduction). In a still further embodiment, such modulation can alter the nature of the interaction between a ligand and its cognate receptor so as to alter the nature of the elicited signal transduction. For example, the molecules can, by binding to the ligand or receptor, alter the ability of such molecules to bind to other ligands or receptors and thereby alter their overall activity. Preferably, such modulation will provide at least a 10% change in a measurable immune system activity, more preferably, at least a 50% change in such activity, or at least a 2-fold, 5-fold, 10-fold, or still more preferably, at least a 100-fold change in such activity.

The term “substantially,” as used in the context of binding or exhibited effect, is intended to denote that the observed effect is physiologically or therapeutically relevant. Thus, for example, a molecule is able to substantially block an activity of a ligand or receptor if the extent of blockage is physiologically or therapeutically relevant (for example if such extent is greater than 60% complete, greater than 70% complete, greater than 75% complete, greater than 80% complete, greater than 85% complete, greater than 90% complete, greater than 95% complete, or greater than 97% complete). Similarly, a molecule is said to have substantially the same immunospecificity and/or characteristic as another molecule, if such immunospecificities and characteristics are greater than 60% identical, greater than 70% identical, greater than 75% identical, greater than 80% identical, greater than 85% identical, greater than 90% identical, greater than 95% identical, or greater than 97% identical).

As used herein, the “co-stimulatory” signals encompass positive co-stimulatory signals (e.g., signals that result in enhancing an activity) and negative co-stimulatory signals (e.g., signals that result in inhibiting an activity).

As used herein, the term “derivative” refers to an antibody or antigen-binding fragment thereof that immunospecifically binds to the same target of a parent or reference antibody but which differs in amino acid sequence from the parent or reference antibody or antigen binding fragment thereof by including one, two, three, four, five or more amino acid substitutions, additions, deletions or modifications relative to the parent or reference antibody or antigen binding fragment thereof. Preferably such derivatives will have substantially the same immunospecificity and/or characteristics, or the same immunospecificity and characteristics as the parent or reference antibody or antigen binding fragment thereof. The amino acid substitutions or additions of such derivatives can include naturally occurring (i.e., DNA-encoded) or non-naturally occurring amino acid residues. The term “derivative” encompasses, for example, chimeric or humanized variants, as well as variants having altered CH1, hinge, CH2, CH3 or CH4 regions, so as to form, for example antibodies, etc., having variant Fc regions that exhibit enhanced or impaired effector or binding characteristics.

As used herein, a “chimeric antibody” is a molecule in which different portions of the antibody are derived from different immunoglobulin molecules such as antibodies having a variable region derived from a non-human antibody and a human immunoglobulin constant region.

As used herein, the term “humanized antibody” refers to an immunoglobulin including a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they should be substantially identical to human immunoglobulin constant regions, i.e., at least about 85-99%, preferably about 95% or more identical. Hence, all parts of a humanized immunoglobulin, except possibly the CDR's, are substantially identical to corresponding parts of natural human immunoglobulin sequences. A humanized antibody is an antibody including a humanized light chain and a humanized heavy chain immunoglobulin. For example, a humanized antibody would not encompass a typical chimeric antibody, because, e.g., the entire variable region of a chimeric antibody is non-human.

As used herein, the term “endogenous concentration” refers to the level at which a molecule is natively expressed (i.e., in the absence of expression vectors or recombinant promoters) by a cell (which cell can be a normal cell, a cancer cell or an infected cell).

As used herein, the terms “treat,” “treating,” “treatment” and “therapeutic use” refer to the elimination, reduction or amelioration of one or more symptoms of a disease or disorder exacerbated by Siglec-15 or a ligand thereof.

As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease.

As used herein, the term “prophylactic agent” refers to an agent that can be used in the prevention of a disorder or disease prior to the detection of any symptoms of such disorder or disease. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, the term “cancer” refers to a neoplasm or tumor resulting from abnormal uncontrolled growth of cells. As used herein, cancer explicitly includes, leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or web-like matrices in a three-dimensional basement membrane or extracellular matrix preparation. Non-cancer cells do not form colonies in soft agar and form distinct sphere-like structures in three-dimensional basement membrane or extracellular matrix preparations.

As used herein, an “immune cell” refers to any cell from the hemopoietic origin including, but not limited to, T cells, B cells, monocytes, dendritic cells, and macrophages.

As used herein, “valency” refers to the number of binding sites available per molecule.

As used herein, the terms “immunologic,” “immunological” or “immune” response is the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, an “immunogenic agent” or “immunogen” is capable of inducing an immunological response against itself on administration to a mammal, optionally in conjunction with an adjuvant.

As used herein, the terms “individual,” “host,” “subject,” and “patient” are used interchangeably herein, and refer to a mammal, including, but not limited to, humans, rodents, such as mice and rats, and other laboratory animals.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “antigenic determinant” and “epitope” are used interchangeably and refer to the structure recognized by an antibody.

As used herein, a “conformational epitope” is an epitope that includes discontinuous sections of the antigen's amino acid sequence. Antibodies bind a conformational epitope based on 3-D surface features, shape, or tertiary structure of the antigen.

As used herein, a “linear epitope” is an epitope that formed by a continuous sequence of amino acids from the antigen. Linear epitopes typically include about 5 to about 10 continuous amino acid residues. Antibodies bind a linear epitope based on the primary sequence of the antigen.

As used herein, a “paratope,” also called an “antigen-binding site,” is a part of an antibody which recognizes and binds to an antigen.

### II. Compositions

### A. Current Pharmacological Interventions in SCI-Related Bone Loss

### 1. Anti-Resorptive Agents

a. Bisphosphonates (BPs)

BPs reduce osteoclast viability, number, and ability to resorb bone and have been found to increase bone mass and reduce fractures in postmenopausal women and stroke survivors (Iwamoto et al, Curr Med Res Opin, 24(5):1379-84 (2008); Shen et al, Bone, 21(1):71-8 (1997)). They are now appreciated to have an additional mechanism of action by regulating the activity of connexin 43 hemichannels on osteocytes and osteoblasts (Brouwers et al, J Orthop Res, 27(11):1521-7 (2009)). Several trials have evaluated the effect of BPs administration on bone loss after acute SCI (Bauman et al, J Rehabil Res Dev, 42(3):305-13 (2005); Gilchrist et al, J Clin Endocrinol Metab, 92(4):1385-90 (2007); Bubbear et al, Osteoporos Int, 22(1):271-9 (2011)). Treatment with BPs, at best, slow bone loss at some sites after acute motor-complete SCI but do not spare bone at the most clinically relevant sublesional locations. Bauman et al. reported that subjects with motor-complete SCI who were administered pamidronate (Bauman et al, J Rehabil Res Dev, (2005)) or zoledronic acid (Bauman et al, PM&R, 7(2):188-201 (2015)) did not preserve BMD at the knee (the distal femur and proximal tibia), the skeletal site which is at highest risk of fracture. Although BPs suppress osteoclast activity, they also potently inhibit osteoblast activity, ultimately leading to reduced bone formation (Jensen et al, Bone, 145:115850-60 (2021)). This dual inhibition on osteoblasts and osteoclasts is likely why the efficacy of BPs has been significantly limited in treatment of SCI. The use of BPs is also associated with a number of side effects that are similar to the other anti-resorptive agent-RANKL antibody, as discussed in greater details below.

b. RANKL Inhibitor

Denosumab, a human monoclonal antibody (Ab) to receptor activator of nuclear factor kB ligand (RANKL), represents an immunopharmacological approach to the treatment of osteoporosis which has been approved by the FDA. The mechanism of action of denosamub, which is to prevent the recruitment and the development of osteoclasts, is distinctly different from that of BPs. Denosamub has been demonstrated to be an effective agent in PMO in several clinical trials, including the Fracture Reduction Evaluation of Denosumab in Osteoporosis Every 6 Months Trial (Bone et al, 2015 ASBMR Annual Meeting; LB-1157 (2015)). Histomorphometric findings suggest that the effects of denosumab on bone remodeling are more potent than those with BPs (Kendler et al, J Bone Miner Res, 25(1):72-81 (2010); Reid et al, J Bone Miner Res, 25(10):2256-65 (2010)). Emerging findings from our clinical investigators have shown that denosumab greatly reduced bone loss after subacute SCI (Cirnigliaro et al, JBMR Plus, (2020)). While these findings are quite promising, denosumab also potently suppresses osteoblast activity and bone formation when it inhibits the activity of osteoclasts (Gifre et al, Osteoporos Int, 27(1):405-10 (2016)). The coupled inhibition of bone resorption and formation is believed to be responsible for the rare, but devastating, side effects of denosumab and BPs [e.g., osteonecrosis of the jaw (ONJ) and atypical femoral fractures (AFF) (Reid et al, J Bone Miner Res (2010); Park-Wyllie et al, JAMA, 305(8):783-9 (2011); Lewiecki, Ther Adv Musculoskele Dis, 10(11):209-23 (2018)). In addition, patients prescribed denosumab have also reported trouble breathing, backache, and an ill-defined pain in muscle and/or bone (Khosla and Hofbauer, Lancet Diab Endocrinol, 5(11):898-907 (2017)). Recent evidence suggests that the cessation of denosumab might be accompanied by a period of accelerated bone resorption and increased fracture risk (McDonald et al, Cell, 184(7):1940 (2021)). Whether these adverse effects will be a concern in SCI remains to be seen, nonetheless the efficacy of denosumab leaves significant room for improvement. Thus, safety limitations coupled with the questionable efficacy of current anti-resorptive agents in treating bone point to the need for novel therapies to treat osteoporosis, including disuse osteoporosis and SCI-related osteoporosis (Bauman et al, J Rehabil Res Dev, 2005; Gilchrist et al, J Clin Endocrinol Metab, 2007; Bauman et al, J Bone Miner Metab, 33(4):410-21 (2015)) including disuse osteoporosis, SCI.

### 2. Bone Anabolic Agents

a. Teriparatide and Abaloparatide

Currently, there are three FDA-approved agents with bone anabolic activity: teriparatide, abaloparatide and romosozumab (Romo, a Sclerostin inhibitor is discussed later). Teriparatide is a peptide containing amino acid residues 1-34 of parathyroid hormone (PTH) that is effective in reducing risk of fractures in post-menopausal osteoporosis (Murad et al, J Clin Endocrinol Metab, 97(6):1871-80 (2012); Freemantle et al, Osteoporos Int, Epub 2012/07/27 (2012)). However, two recent studies did not observe significant changes in BMD of the lower extremities of individuals with SCI after 6 months of teriparatide coupled with mechanical loading or vibration (Gordon et al, PM&R, 5(8):663-71 (2013); Edwards et al, J Bone Miner Res, 33(10):1729-40 (2018)), suggesting a lack of efficacy of teriparatide in the treatment of bone loss after SCI. Another potential concern is that even if effective regimens with teriparatide and abaloparatide (PTH-related protein analog) could be worked out over time, treatment would be limited to a 2-year period in patients given the increased potential risk of developing osteosarcoma with longer duration of therapy (Cosman et al, N Engl J Med, 353(6):566-75 (2005); Zhang et al, Nat Med, 18(2):307-14 (2012)).

b. Sclerostin Inhibitors

A monoclonal human antibody against sclerostin that targets the Wnt signaling pathway, Romo, has been recently approved as a new and potent anabolic agent for the treatment of women with post-menopausal osteoporosis (PMO) at high risk for fracture. In women with PMO, Romo can increase BMD and bone formation while decreasing bone resorption (McClung et al, N Engl J Med, 370(17):1664-5 (2014)). Romo significantly reduced the incidence of new vertebral fractures in the FRAME study, but the drug failed to meet the secondary objective of reducing the incidence of non-vertebral fractures (Cosman et al, N Engl J Med, 375(16):1532-43 (2016)). However, Romo significantly reduced non-vertebral fractures by 19% in a separate study. Importantly, this study also reported that patients on Romo had a slightly higher risk of serious cardiovascular events than the comparator drug (alendronate, a BP) (Saag et al, N Engl J Med, 377(15):1417-27 (2017)), a risk that is still under debate (Cummings et al, Osteoporos Int, 31(6):1019-21 (2020)). Recent preclinical work strongly supports sclerostin antibody as an attractive agent to decrease bone loss after acute and chronic SCI (Qin et al, J Bone Miner Res, 30(11):1994-2004 (2015); Qin et al, Osteoporos Int, 27(12):3627-36 (2016); Zhao et al, Calif Tiss Int, 103(4):443-54 (2018)). However, the use of the drug has been limited to one year by the FDA and carries a black box warning from the FDA cautioning its use in patients at higher risk for cardiovascular disease and stroke, two risk factors that are greatly elevated in SCI patients (Cragg et al, Neurology, 81(8):723-8 (2013)). When considering the agent's uncertain efficacy to reduce non-vertebral fractures, the possibility of increased cardiovascular side effects, and relatively short period of safe therapeutic administration, development of an alternative therapeutic approach is necessary. The most frequent fracture in patients with SCI occurs at the distal femur and proximal tibia (e.g., the knee region), which is a non-vertebral site. SCI is an ongoing, life-long condition, and, as such, bone loss that follows SCI is a process that continues for decades (Qin et al, Ann NY Acad Sci, 2010; Bauman et al, Osteoporos Int, 1999). Thus, even if Romo proves to be efficacious, this agent might not fully meet the needs for the treatment of neurogenic bone loss in those with chronic SCI. Similarly, osteoporosis due to other reduced load-bearing and non-ambulatory states are medical conditions that still require more effective therapeutics. The availability of an agent that will reduce non-vertebral fractures and be safely administered over a longer period of time would be of high clinical relevance to improve long-term bone health in the aforementioned patients.

### B. Siglec-15—An Agent for Improving Skeletal Integrity

The anti-Siglec-15 Ab is a novel, potent and potentially safer anti-resorptive and anabolic agent for use in improving skeletal integrity after osteoporosis induced by reduced mechanical loading. Anti-Siglec-15 antibodies or their antigen-binding fragments that specifically bind human Siglec-15 can inhibit osteoclast maturation and activity while preserving osteoblast function, a feature distinct from the current antiresorptive agents (e.g., RANKL Ab and biophosphates (BPs)) that inhibit the activity of both osteoclasts and osteoblasts and the questionable efficacy of BPs in treating bone disease in those with SCI. Thus, Siglec-15 Ab can be more potent and potentially safer than strictly anti-resorptive agents. In addition, the potentially improved safety profile of Siglec-15 Ab, as evidenced in preclinical toxicology studies with our anti-S15 mAb and given safety profile for several other Siglec-15 mAbs that have advanced in the clinic to date, should for long-term treatment of patients with SCI, unlike bone anabolic agents (teriparatide, abaloparatide, and romosozumab), which are limited in treatment length of 1 to 2 years due to the safety concerns or other adverse effects. This invention provides methods of use of anti-Siglec-15 antibodies to mitigate bone loss in immobilizing conditions, such as SCI, with applications that should extend to other reduced mechanical loading conditions that result in osteoporosis.

Collectively, there are two major advantages of using Siglec-15 Ab for neurogenic bone loss after SCI. First, the unique and favorable features of Siglec-15 neutralizing Ab, as described earlier, make it distinct from the current antiresorptive agents RANKL Ab and BPs that inhibit the activity of both osteoclasts and osteoblasts. As such, Siglec-15 Ab is capable of offering better efficacy than that of BPs and RANKL Ab in SCI-induced bone loss because Siglec-15 Ab prevents bone loss and promotes bone formation, whereas BPs and RANKL Ab result in suppression of bone formation, an effect that may have resulted in questionable efficacy of BPs in treating bone disease in those with SCI (Bauman et al, J Rehabil Res Dev, (2005); Gilcrhist et al, J Clin Endocrinol Metab, (2007); Bauman et al, J Bone Miner Metab, 33(4):410-21 (2015)).

Sialic-acid-binding immunoglobulin-like lectin (Siglec)-15 is a cell surface receptor that regulates the function of immune cells through glycan recognition. Siglec-15 is expressed on a subset of osteoclast precursor cells and is highly expressed on mature osteoclasts (Humphrey et al, Clin Rev Allergy Immunol, 51(1):48-58 (2016); Kameda et al, Bone, 71:217-26 (2015); Zhu et al, Immunity, 34(4):466-78 (2011)). When pre-cursor cells begin to differentiate toward mature osteoclasts in the presence of RANKL, Siglec-15 forms a complex with the adaptor protein DAP12 to promote osteoclast fusion (Stuible et al, J Biol Chem, 2014; Ishida-Kitagawa et al, J Biol Chem, 2012). This role of Siglec-15 as a promoter of osteoclast fusion is further demonstrated by the Siglec-15 knock-out mice, which display a mild osteopetrotic phenotype and reduced multinucleated osteoclasts. These data suggest that the absence or blocking of Siglec-15 can increase bone formation while simultaneously decreasing bone resorption (Hiruma et al, Bone, 2013).

These unique and favorable features make a blocking antibody against Siglec-15 distinct from current anti-resorptive agents that inhibit the activity of both osteoclasts and osteoblasts, as well as that of other bone anabolic agents that have major safety concerns with long-term use. For these reasons, Siglec-15 Ab holds greater promise to improve skeletal health in Veterans or other individuals with chronic SCI than that of currently available agents.

The anti-Siglec-15 monoclonal antibody has been tested in an acute SCI model to support the use as a new treatment option for osteoporosis as a result of non-ambulatory states. To this end, 12-week-old male Wistar rats underwent complete spinal cord transection. Immediately after SCI, the rats were treated with either vehicle or anti-Siglec-15 antibody at 20 mg/kg once every 2 weeks for 8 weeks until euthanasia. Treatment with the anti-Siglec-15 antibody almost completely prevented sublesional bone loss and preserved trabecular micro-architecture and bone strength after acute SCI in rats (Peng et al, ABMR 2021 Annual Meeting VPL-415). Mechanistically, anti-Siglec-15 antibody significantly suppressed osteoclast maturation and bone resorption while increasing osteoblastogenesis to maintain bone formation for a net anabolic outcome.

### C. Siglec-15 Sequences

Sialic acid-binding Ig-like lectin 15 (“Siglec-15”, also referred to as CD33 antigen-like 3, and CD33L3) is a type 1 transmembrane protein expressed on macrophages and/or dendritic cells of human spleen and lymph nodes (Angata, et al., Glycobiology, 17(8):838-46 (2007), which is specifically incorporated by reference herein in its entirety). The extracellular domain of Siglec-15 binds to sialylated glycoproteins and preferentially recognizes the Neu5Aca2-6GalNAcα-structure.

Siglec-15 associates with the activating adaptor proteins DNAX activation protein (DAP)12 and DAP10 via its lysine residue (residue K274) in the transmembrane domain, indicating that it functions as an activating signaling molecule. Orthologs of Siglec-15 are present not only in mammals but also in other branches of vertebrates, and believed to play a conserved, regulatory role in the immune system of vertebrates.

Siglec-15 directly regulates T cell function by inhibiting T cell proliferation and proinflammatory cytokine production. Siglec-15 indirectly affects T cell function via myeloid cells. Siglec-15 expressed on tumor cells or M2 macrophages interacts with its binding partner on myeloid cells providing survival and differentiation signal resulting in a unique myeloid cell population that produces TNF-α, IL-6 and IL-1β. The secreted cytokines further promote tumor growth. This subset of myeloid cells may affect T cell function by reducing IFN-7 production in T cells.

Amino acid sequences for human Siglec-15 are known in the art and include, for example,

UniProtKB - Q6ZMC9 (SIG15_HUMAN),
(SEQ ID NO: 1)
MEKSIWLLACLAWVLPTGSFVRTKIDTTENLLNTEVHSSPAQRWSMQVPP
EVSAEAGDAAVLPCTFTHPHRHYDGPLTAIWRAGEPYAGPQVERCAAARG
SELCQTALSLHGRFRLLGNPRRNDLSLRVERLALADDRRYFCRVEFAGDV
HDRYESRHGVRLHVTAA

[symbol]

[symbol]

[symbol]

[symbol]

[symbol]

LFRFHGASGASTVALLLGALGFKALLLLGVLAARAARRRPEHLDTPDTPP
RSQAQESNYENLSQMNPRSPPATMCSP,

and which is specifically incorporated by reference in its entirety.

Human Siglec-15 includes a signal peptide sequence from amino acids 1-19 of SEQ ID NO:1, an extracellular domain from amino acids 20-263 of SEQ ID NO:1 (illustrated with bold and italic lettering), a transmembrane domain from amino acids 264-284 of SEQ ID NO:1, and a cytoplasmic domain from amino acids 285-328 of SEQ ID NO:1. The Ig-like V-type domain is predicted to be from amino acids 40-158 of SEQ ID NO:1 (illustrated with single underlining) and the Ig-like C2-type domain is predicted to be from amino acids 168-251 of SEQ ID NO:1 (illustrated with double underlining). Disulfide bonds are believed to form between residues 64 and 142; 95 and 104; and 187 and 237, and glycosylation is predicted at residue 172. Amino acids 276-279 has been referred to as a poly-leucine domain. A known variant is a F273L substitution variant.

Amino acid sequences for mouse Siglec-15 are known in the art and include, for example,

(SEQ ID NO: 2)
MEGSLQLLACLACVLQMGSLVKTRRDASGDLLNTEAHSAPAQRWSMQVPA
EVNAEAGDAAVLPCTFTHPHRHYDGPLTAIWRSGEPYAGPQVERCTAAPG
SELCQTALSLHGRFRLLGNPRRNDLSLRVERLALADSGRYFCRVEFTGDA
HDRYESRHGVRLRVTAAA

[symbol]

[symbol]

[symbol]

[symbol]

[symbol]

[symbol]

LFRFHGAPGTSTLALLLGALGLKALLLLGILGA
RATRRRLDHLVPQDTPPRSQAQESNYENLSQMSPPGHQLPRVCCEELLSH
HHLVIHHEK,

UniProtKB—A7E1W8 (A7E1W8_MOUSE), and which is specifically incorporated by reference in its entirety.

Mouse Siglec-15 includes a signal peptide sequence from amino acids 1-23 of SEQ ID NO:2, an extracellular domain from amino acids 24-262 of SEQ ID NO:2 (illustrated with bold and italic lettering), a transmembrane domain from amino acids 263-283 of SEQ ID NO:2, and a cytoplasmic domain from amino acids 284-342 of SEQ ID NO:2. The Ig-like V-type domain is predicted to be from amino acids 40-145 of SEQ ID NO:2 (illustrated with single underlining) and the Ig-like C2-type domain is predicted to be from amino acids 169-250 of SEQ ID NO:2 (illustrated with double underlining).

### D. Siglec-15-Binding Molecules

Siglec-15-binding molecules, such as antibodies and antigen binding fragments thereof and other polypeptides that bind to Siglec-15 are provided. The sequences of the heavy and light chain variable regions, and CDRs thereof, from mouse anti-Siglec-15 antibodies are provided below. Antibodies, antigen binding fragments and other polypeptides including one or more of the sequences below, and variants thereof are provided. For example, antibodies, antigen binding fragments, and polypeptides including one, two, or three CDRs of an anti-Siglec-15 antibody light chain variable region and/or one, two, or three CDRs of an anti-Siglec-15 antibody heavy chain variable region that bind to Siglec-15 are provided. In some embodiments, the antibodies, antigen binding fragments, and polypeptides include the light chain variable region of an anti-Siglec-15 antibody, the heavy chain variable region of an anti-Siglec-15, or a combination thereof, and can bind to Siglec-15.

For example, the disclosed molecules can immunospecifically bind to Siglec-15 (e.g., SEQ ID NO:1, SEQ ID NO:2, etc.). For example, molecules are provided that can immunospecifically bind to human Siglec-15:
(I) arrayed on the surface of a cell (preferably a live cell);
(II) arrayed on the surface of a cell (preferably a live cell) at an endogenous concentration;
(III) arrayed on the surface of a live cell, and modulates binding between Siglec-15 (e.g., SEQ ID NO:1, SEQ ID NO:2, etc.) and Neu5Aca2-6GalNAcα, LRRC4C, an Siglec-15-counter-receptor (S15-CR), or a combination thereof;
(IV) arrayed on the surface of a live cell, and reduces, prevents, or inhibits TGF-β secretion;
(V) arrayed on the surface of a live cell, wherein the cell is a myeloid cell such as a macrophage or dendritic cell, or a cancer cell (e.g., brain cancer cell, renal cell carcinoma cell (RCC), Ewing sarcoma cell, breast cancer cell, or ovarian cancer cell);
(VI) combinations thereof.

### 1. Mouse Anti-Human Siglec-15 Antibody Sequences

As described in the Examples below, Siglec-15 knock out mice (n=2) were immunized with hS15.mIg (human Siglec 15 extracellular domain [ECD] fused with mouse IgG2a) emulsified with CFA (complete Freund adjuvant) to generate a panel of mouse anti-human Siglec-15 mAbs.

The sequences of light and heavy chain variable regions for monoclonal antibodies produced by hybridomas, referred to herein as 1H3 and 5G12 are provided below. CDRs are underlined and bolded in the context of the light and heavy chain sequences.

### a. 1H3 Sequences:

i. Light Chain

1H3 Light Chain Variable Region Amino Acid Sequence is:

(SEQ ID NO: 3)
DIQMTQASSSLSVSLGGRVTITCKASDHINNWLAWYQQKPGNAPRLLISG
ATSLETGVPSRFSGSGSGKDYTLSITSLQTEDVATYYCQQYWSSPLTFGA
GTKLELK,

with

| | 1H3 Light Chain CDR1: |
|---|---|
| (SEQ ID NO: 4) | |
| | KASDHINNWLA |
| | 1H3 Light Chain CDR2: |
| (SEQ ID NO: 5) | |
| | GATSLET |
| | 1H3 Light Chain CDR3: |
| (SEQ ID NO: 6) | |
| | QQYWSSPLT. |

A nucleic acid sequence encoding the 1H3 light chain variable region is:

(SEQ ID NO: 7)
GACATCCAGATGACACAGGCTTCATCCTCCTTGTCTGTATCTCTAGGAGG
CAGAGTCACCATTACTTGCAAGGCAAGTGACCACATTAATAATTGGTTGG
CCTGGTATCAGCAGAAACCAGGAAATGCTCCTAGGCTCTTAATATCTGGT
GCAACCAGTTTGGAAACTGGGGTTCCTTCAAGATTCAGTGGCAGTGGATC
TGGAAAGGATTACACTCTCAGCATTACCAGTCTTCAGACTGAAGATGTTG
CTACTTATTACTGTCAACAGTATTGGAGTTCTCCTCTCACGTTCGGTGCT
GGGACCAAGCTGGAGCTGAAA.

ii. Humanized Light Chain
aa. 1H3 hVL1

One embodiment provides a humanized anti-SIGLEC-15 antibody 1H3 hVL1 having a variable light chain amino acid sequence of

(SEQ ID NO: 8)
DIQMTQSPSSLSASVGDRVTITCKASDHINNWLAWYQQKPGKAPKLLISG
ATSLETGVPSRFSGSGSGTDYTFTISSLQPEDIATYYCQQYWSSPLTFGG
GTKVEIK

with

| | 1H3 hVL1 CDR1: |
|---|---|
| (SEQ ID NO: 4) | |
| | KASDHINNWLA |
| | 1H3 hVL1 CDR2: |
| (SEQ ID NO: 5) | |
| | GATSLET |
| | 1H3 hVL1 CDR3: |
| (SEQ ID NO: 6) | |
| | QQYWSSPLT. |

The underlined amino acids are changed with regard to the parent sequence.

bb. 1H3 hVL2

Another embodiment provides a humanized anti-SIGLEC-15 1H3 hVL2 antibody having a variable light chain amino acid sequence of

(SEQ ID NO: 9)
DIQMTQSPSSLSASVGDRVTITCKASDHINNWLAWYQQKPGKVPKLLISG
ATSLETGVPSRFSGSGSGTDYTLTISSLQPEDVATYYCQQYWSSPLTFGG
GTKVEIK

with

| | 1H3 hVL2 CDR1: |
|---|---|
| (SEQ ID NO: 4) | |
| | KASDHINNWLA |
| | 1H3 hVL2 CDR2: |
| (SEQ ID NO: 5) | |
| | GATSLET |
| | 1H3 hVL2 CDR3: |
| (SEQ ID NO: 6) | |
| | QQYWSSPLT. |

The underlined amino acids are changed with regard to the parent sequence.

cc. 1H3 hVL3

Still another embodiment provides a humanized anti-SIGLEC-15 1H3 hVL3 antibody having a variable light chain amino acid sequence of

(SEQ ID NO: 10)
DIQMTQSPSSLSASVGDRVTITCKASDHINNWLAWYQQKPGKAPKLLISG
ATSLETGVPSRFSGSGSGTDYTLTISSLQPEDVATYYCQQYWSSPLTFGG
GTKVEIK

with

| | 1H3 hVL3 CDR1: |
|---|---|
| (SEQ ID NO: 4) | |
| | KASDHINNWLA |
| | 1H3 hVL3 CDR2: |
| (SEQ ID NO: 5) | |
| | GATSLET |
| | 1H3 hVL3 CDR3: |
| (SEQ ID NO: 6) | |
| | QQYWSSPLT. |

The underlined amino acids are changed with regard to the parent sequence.

iii. Heavy Chain

1H3 Heavy Chain Variable Region Amino Acid Sequence is:

(SEQ ID NO: 11)
QVQLKESGPGLVAPSQSLSITCTVSGFSLSNYGVHWVRQPPGKGLEWLVL
IWSDGSTTYNSALKSRLSISKDNSKSQVFLKMNSLQTGDTAMYYCARHPY
DDYSGYYYTMDYWGQGTSVTVSS,

with

(SEQ ID NO: 12)
1H3 Heavy Chain CDR1: NYGVH
(SEQ ID NO: 13)
1H3 Heavy Chain CDR2: LIWSDGSTTYNSALKS
(SEQ ID NO: 14)
1H3 Heavy Chain CDR3: HPYDDYSGYYYTMDY.

A nucleic acid sequence encoding the 1H3 heavy chain variable region is:

(SEQ ID NO: 15)
CAGGTGCAGCTGAAGGAGTCAGGACCTGGCCTGGTGGCGCCCTCACAGAG
CCTGTCCATCACATGCACCGTCTCAGGGTTCTCATTAAGCAATTATGGTG
TACACTGGGTTCGCCAGCCTCCAGGAAAGGGTCTGGAGTGGCTGGTACTG
ATATGGAGTGATGGAAGCACAACCTATAATTCAGCTCTCAAATCCAGACT
GAGCATCAGCAAGGACAACTCCAAGAGCCAAGTTTTCTTAAAAATGAACA
GTCTCCAAACTGGTGACACAGCCATGTACTACTGTGCCAGACATCCCTAT
GATGATTATTCCGGCTATTACTATACTATGGACTACTGGGGTCAAGGAAC
CTCAGTCACCGTCTCCTCA.

iv. Humanized Heavy Chains
aa. 1H3 hVH1

One embodiment provides a humanized anti-SIGLEC 15 antibody 1H3 hVH1 having a variable heavy chain amino acid sequence of

(SEQ ID NO: 16)
QVQLQESGPGLVKPSETLSLTCTVSGFSLSNYGVHWVRQPPGKGLEWIVL
IWSDGSTTYNSALKSRVTISKDTSKNQVSLKLSSVTAADTAVYYCARHPY
DDYSGYYYTMDYWGQGTLVTVSS

with

(SEQ ID NO: 12)
1H3 hVH1 CDR1: NYGVH
(SEQ ID NO: 13)
1H3 hVH1 CDR2: LIWSDGSTTYNSALKS
(SEQ ID NO: 14)
1H3 hVH1 CDR3: HPYDDYSGYYYTMDY.

The underlined amino acids are changed with regard to the parent sequence.

bb. 1H3 hVH2

Another embodiment provides a humanized anti-SIGLEC 15 1H3 hVH2 antibody having a variable heavy chain amino acid sequence of

(SEQ ID NO: 17)
QVQLQESGPGLVKPSETLSLTCTVSGFSLSNYGVHWVRQPPGKGLEWIGL
IWSDGSTTYASALKSRVTISKDTSKNQVSLKLSSVTAADTAVYYCARHPY
DDYSGYYYTMDYWGQGTLVTVS

with

(SEQ ID NO: 12)
1H3 hVH2 CDR1: NYGVH
(SEQ ID NO: 18)
1H3 hVH2 CDR2: LIWSDGSTTYASALKS
(SEQ ID NO: 14)
1H3 hVH2 CDR3: HPYDDYSGYYYTMDY.

The underlined amino acids are changed with regard to the parent sequence.

cc. 1H3 hVH3

Still another embodiment provides a humanized anti-SIGLEC 15 1H3 hVH3 antibody having a variable heavy chain amino acid sequence of

(SEQ ID NO: 19)
QVQLQESGPGLVKPSETLSLTCTVSGFSLSNYGVHWVRQPPGKGLEWIGL
IWSDGSTTYNPSLKSRVTISKDTSKNQVSLKLSSVTAADTAVYYCARHPY
DDYSGYYYTMDYWGQGTLVTVS

with

1H3 hVH3 CDR1:
(SEQ ID NO: 12)
NYGVH
1H3 hVH3 CDR2:
(SEQ ID NO: 20)
LIWSDGSTTYNPSLKS
1H3 hVH3 CDR3:
(SEQ ID NO: 14)
HPYDDYSGYYYTMDY.

The underlined amino acids are changed with regard to the parent sequence.

dd. 1H3 hVH4

Another embodiment provides a humanized anti-SIGLEC 15 antibody 1H3 hVH4 having a variable heavy chain amino acid sequence of

(SEQ ID NO: 21)
QVQLQESGPGLVKPSETLSLTCTVSGFSLSNYGVHWVRQPPGKGLEWIGL
IWSEGSTTYASALKSRVTISKDTSKNQVSLKLSSVTAADTAVYYCARHPY
DDYSGYYYTMDYWGQGTLVTVS

with

(SEQ ID NO: 12)
1H3 hVH4 CDR1: NYGVH
(SEQ ID NO: 22)
1H3 hVH4 CDR2: LIWSEGSTTYASALKS
(SEQ ID NO: 14)
1H3 hVH4 CDR3: HPYDDYSGYYYTMDY.

### b. 5G12 Sequences:

i. Light Chain

5G12 Light Chain Variable Region Amino Acid Sequence is:

(SEQ ID NO: 23)
DIKMTQSPSSMYASLGERVTITCKASQDINSYLSWFQQKPGKSPK
TLIYRANRLVDGVPSRFSGSGSGQDYSLTISSLEYEDMGIYYCLQ
YDEFPYTFGGGTKLEIKR,

with

(SEQ ID NO: 24)
5G12 Light Chain CDR1: KASQDINSYLS
(SEQ ID NO: 25)
5G12 Light Chain CDR2: RANRLVD
(SEQ ID NO: 26)
5G12 Light Chain CDR3: LQYDEFPYT

A nucleic acid sequence encoding the 5G12 light chain variable region is:

(SEQ ID NO: 27)
GACATCAAGATGACCCAGTCTCCATCTTCCATGTATGCATCTCTAGGAGA
GAGAGTCACTATCACTTGCAAGGCGAGTCAGGACATTAATAGCTATTTAA
GCTGGTTCCAGCAGAAACCAGGGAAATCTCCTAAGACCCTGATCTATCGT
GCAAACAGATTGGTAGATGGGGTCCCATCAAGGTTCAGTGGCAGTGGATC
TGGGCAAGATTATTCTCTCACCATCAGCAGCCTGGAGTATGAAGATATGG
GAATTTATTATTGTCTACAGTATGATGAGTTTCCGTACACGTTCGGAGGG
GGGACCAAGCTGGAAATAAAA.

ii. Humanized Light Chain

Clone 5G12 was humanized providing three humanized heavy chains and five humanized light chains

aa. 5G12 hVL1

One embodiment provides a humanized anti-SIGLEC-15 antibody 5G12 hVL1 having a variable light chain amino acid sequence of

(SEQ ID NO: 28)
DIQMTQSPSSLSASVGDRVTITCKASQDINSYLSWFQQKPGKAPKTLIYR
ANRLVDGVPSRFSGSGSGTDYTLTISSLQPEDFATYYCLQYDEFPYTFGG
GTKVEIK

(SEQ ID NO: 24)
CDR1 of 5G12 hVL1: KASQDINSYLS
(SEQ ID NO: 25)
CDR2 of 5G12 hVL1: RANRLVD
(SEQ ID NO: 26)
CDR3 of 5G12 hVL1: LQYDEFPYT

bb. 5G12 hVL2

Another embodiment provides a humanized anti-SIGLEC-15 antibody 5G12 hVL2 having a variable light chain amino acid sequence of

(SEQ ID NO: 29)
DIQMTQSPSSLSASVGDRVTITCKASQDINTYLSWFQQKPGKAPK
TLIYRANRLVDGVPSRFSGSGSGTDYTLTISSLQPEDFATYYCLQ
YDEFPYTFGGGTKVEIK

CDR1 of 5G12 hVL2:
(SEQ ID NO: 30)
KASQDINTYLS
CDR2 of 5G12 hVL2:
(SEQ ID NO: 25)
RANRLVD
CDR3 of 5G12 hVL2:
(SEQ ID NO: 26)
LQYDEFPYT

cc. 5G12 hVL3

Another embodiment provides a humanized anti-SIGLEC-15 antibody 5G12 hVL3 having a variable light chain amino acid sequence of

(SEQ ID NO: 31)
DIQMTQSPSSLSASVGDRVTITCKASQDINVYLSWFQQKPGKAPK
TLIYRANRLVDGVPSRFSGSGSGTDYTLTISSLQPEDFATYYCLQ
YDEFPYTFGGGTKVEIK

CDR1 of 5G12 hVL3:
(SEQ ID NO: 32)
KASQDINVYLS
CDR2 of 5G12 hVL3:
(SEQ ID NO: 25)
RANRLVD
CDR3 of 5G12 hVL3:
(SEQ ID NO: 26)
LQYDEFPYT

dd. 5G12 hVL4

Another embodiment provides a humanized anti-SIGLEC-15 antibody 5G12 hVL4 having a variable light chain amino acid sequence of

(SEQ ID NO: 33)
DIQMTQSPSSLSASVGDRVTITCKASQDIQSYLSWFQQKPGKAPK
TLIYRANRLVDGVPSRFSGSGSGTDYTLTISSLQPEDFATYYCLQ
YDEFPYTFGGGTKVEIK

CDR1 of 5G12 hVL4:
(SEQ ID NO: 34)
KASQDIQSYLS
CDR2 of 5G12 hVL4:
(SEQ ID NO: 25)
RANRLVD
CDR3 of 5G12 hVL4:
(SEQ ID NO: 26)
LQYDEFPYT

ee. 5G12 hVL5

Another embodiment provides a humanized anti-SIGLEC-15 antibody 5G12 hVL5 having a variable light chain amino acid sequence of

(SEQ ID NO: 35)
DIQMTQSPSSLSASVGDRVTITCKASQDINVYLSWFQQKPGKAPK
TLIYRANRLTSGVPSRFSGSGSGTDYTLTISSLQPEDFATYYCLQ
YDEFPYTFGGGTKVEIK

CDR1 of 5G12 hVL5:
(SEQ ID NO: 32)
KASQDINVYLS
CDR2 of 5G12 hVL5:
(SEQ ID NO: 36)
RANRLTS
CDR3 of 5G12 hVL5:
(SEQ ID NO: 26)
LQYDEFPYT

iii. Heavy Chain

5G12 Heavy Chain Variable Region Amino Acid Sequence is:

(SEQ ID NO: 37)
QVQLQQPGAELVKPGASVKMSCKASGYTFTSYWITWVIQRPGQGL
EWIGDIYCGSDTMHYNEKFKNKATLTVDTSSSTAYMQLSSLTSED
SAVYYCARWWDYGSSYDYFDYWGQGTTLTVSS,

5G12 Heavy Chain CDR1:
(SEQ ID NO: 38)
GYTFTSYWIT
5G12 Heavy Chain CDR2:
(SEQ ID NO: 39)
DIYCGSDTMHYNEKFKN
5G12 Heavy Chain CDR3:
(SEQ ID NO: 40)
WWDYGSSYDYFDY.

A nucleic acid sequence encoding the 5G12 heavy chain variable region is:

(SEQ ID NO: 41)
CAGGTCCAACTGCAGCAGCCTGGGGCTGAGCTTGTGAAGCCTGGG
GCTTCAGTGAAGATGTCCTGCAAGGCTTCTGGCTACACCTTCACC
AGCTACTGGATAACCTGGGTGATACAGAGGCCGGGACAAGGCCTT
GAGTGGATTGGAGATATTTATTGTGGTAGTGATACTATGCACTAC
AATGAGAAGTTCAAGAACAAGGCCACACTGACTGTAGACACATCC
TCCAGCACAGCCTACATGCAGCTCAGCAGCCTGACATCTGAGGAC
TCTGCGGTCTATTACTGTGCAAGATGGTGGGACTACGGTAGTAGC
TACGACTACTTTGACTACTGGGGCCAAGGCACCACTCTCACAGTC
TCCTCA.

iv. Humanized Heavy Chains
aa. 5G12 hVH1

One embodiment provides a humanized anti-SIGLEC 15 antibody having a 5G12 hVH1 variable heavy chain amino acid sequence of

(SEQ ID NO: 42)
QVQLVQSGAEVKKPGASVKVSCKASGYTFTSYWITWVRQAPGQGL
EWMGDIYSGSDTMHYAEKFQGRVTLTVDTSTSTAYMELSSLRSED
TAVYYCARWWDYGSSYDYFDYWGQGTLVTVSS

CDR1 of 5G12 hVH1:
(SEQ ID NO: 43)
SYWIT
CDR2 of 5G12 hVH1:
(SEQ ID NO: 44)
DIYSGSDTMHYAEKFQG
CDR3 of 5G12 hVH1:
(SEQ ID NO: 40)
WWDYGSSYDYFDY

bb. 5G12 hVH2

Another embodiment provides a humanized anti-SIGLEC 15 antibody having a 5G12 hVH2 variable heavy chain amino acid sequence of

(SEQ ID NO: 45)
QVQLVQSGAEVKKPGASVKVSCKASGYTFTSYWITWVRQAPGQGLEWMGD
IYSGSDTTHYAEKFQGRVTLTVDTSTSTAYMELSSLRSEDTAVYYCARWW
DYGSSYDYFDYWGQGTLVTVSS

| | CDR1 of 5G12 hVH2: |
|---|---|
| (SEQ ID NO: 43) | |
| | SYWIT |
| | CDR2 of 5G12 hVH2: |
| (SEQ ID NO: 44) | |
| | DIYSGSDTMHYAEKFQG |
| | CDR3 of 5G12 hVH2: |
| (SEQ ID NO: 40) | |
| | WWDYGSSYDYFDY |

cc. 5G12 hVH3

Another embodiment provides a humanized anti-SIGLEC 15 antibody having a 5G12 hVH3 variable heavy chain amino acid sequence of

(SEQ ID NO: 46)
QVQLVQSGAEVKKPGASVKVSCKASGYTFTSYWISWVRQAPGQGLEWMGD
IYSGSDTTHYAEKFQGRVTLTVDTSTSTAYMELSSLRSEDTAVYYCARWW
DYGSSYDYFDYWGQGTLVTVSS

| | CDR1 of 5G12 hVH3: |
|---|---|
| (SEQ ID NO: 47) | |
| | SYWIS |
| | CDR2 of 5G12 hVH3: |
| (SEQ ID NO: 44) | |
| | DIYSGSDTMHYAEKFQG |
| | CDR3 of 5G12 hVH3: |
| (SEQ ID NO: 40) | |
| | WWDYGSSYDYFDY. |

### 2. Anti-Siglec-15 Antibodies and Antigen Binding Fragments Thereof

Siglec-15 binding molecules, including antibodies and antigen binding fragments thereof, that bind to one or more Siglec-15 polypeptides or fusion proteins, or fragments or variants thereof are disclosed. The antibodies disclosed herein are typically monoclonal antibodies, or antigen binding fragments thereof, that bind to an epitope present on a Siglec-15 polypeptide, or fragment or fusion thereof. In some embodiments the antibody binds to a conformational epitope. In some embodiments the antibody binds to a linear epitope. A linear epitope can be 4, 5, 6, 7, 8, 9, 10, 11, or more continuous amino acids in length. The epitope can include one or more non-amino acid elements, post-translation modifications, or a combination thereof. Examples of post-translational modifications include, but are not limited to glycosylation, phosphorylation, acetylation, citrullination and ubiquitination. For example, antibodies can bind an epitope that is formed at least in-part by one or more sugar groups.

The antibody or antigen binding fragment thereof can bind to an epitope that is present on an endogenous Siglec-15 polypeptide, or a recombinant Siglec-15 polypeptide, or a combination thereof. In some embodiments, the antibody or antigen binding fragment thereof binds to the extracellular domain, or a fragment thereof, or an epitope formed therefrom of Siglec-15. In some embodiments, the antibody or antigen binding fragment thereof is a function blocking antibody that reduces or prevents Siglec-15 from binding to one or more of its ligands, reduces intracellular signaling modulated by Siglec-15, or a combination thereof.

As discussed above, Siglec-15 sialylated glycoproteins and preferentially recognizes the Neu5Aca2-6GalNAcα-structure. Previous studies have shown that Siglec-15 binds to Leucine-rich repeat-containing protein 4C (LRRC4C) (also referred to as Netrin-G1 ligand, and NGL-1), which may be depend or independent of a Neu5Aca2-6GalNAcα-structure. Nucleic acid and polypeptide sequences for LRRC4C are known in the art and include, for example,

MLNKMTLHPQQIMIGPRFNRALFDPLLVVLLALQLLVVAGLVRAQTCPSV
CSCSNQFSKVICVRKNLREVPDGISTNTRLLNLHENQIQIIKVNSFKHLR
HLEILQLSRNHIRTIEIGAFNGLANLNTLELFDNRLTTIPNGAFVYLSKL
KELWLRNNPIESIPSYAFNRIPSLRRLDLGELKRLSYISEGAFEGLSNLR
YLNLAMCNLREIPNLTPLIKLDELDLSGNHLSAIRPGSFQGLMHLQKLWM
IQSQIQVIERNAFDNLQSLVEINLAHNNLTLLPHDLFTPLHHLERIHLHH
NPWNCNCDILWLSWWIKDMAPSNTACCARCNTPPNLKGRYIGELDQNYFT
CYAPVIVEPPADLNVTEGMAAELKCRASTSLTSVSWITPNGTVMTHGAYK
VRIAVLSDGTLNFTNVTVQDTGMYTCMVSNSVGNTTASATLNVTAATTTP
FSYFSTVTVETMEPSQDEARTTDNNVGPTPVVDWETTNVTTSLTPQSTRS
TEKTFTIPVTDINSGIPGIDEVMKTTKIIIGCFVAITLMAAVMLVIFYKM
RKQHHRQNHHAPTRTVEIINVDDEITGDTPMESHLPMPAIEHEHLNHYNS
YKSPFNHTTTVNTINSIHSSVHEPLLIRMNSKDNVQETQI (SEQ ID
NO: 48, UniProtKB - Q9HCJ2 LRC4C_HUMAN and which
is specifically incorporated by reference herein
in its entirety).

Siglec-15 may also bind to a counter-receptor (S15-CR) on immune cells such as T cells.

In some embodiments, binding of the antibody or antigen binding fragment thereof to Siglec-15 can increase immune activation, reduce immune suppression, or a combination thereof. For example, in particular embodiments, the antibody or antigen binding fragment thereof binds to the Ig-like V-type domain or the Ig-like C2-type domain of Siglec-15. In some embodiments, the epitope includes the sialic acid binding site of Siglec-15, (e.g., the epitope include residue 143 of SEQ ID NO:1).

In some embodiments, the antibody binds to part or the all of the same epitope as monoclonal antibody 1H3 or 5G12. The epitope can be a linear epitope or a conformational epitope. In some embodiments, the antibody has the same epitope specificity as monoclonal antibody 1H3 or 5G12. This can be achieved by producing a recombinant antibody that contains the paratope of monoclonal antibody 1H3 or 5G12. In some embodiments, the Siglec-15 binding molecule includes some or all of the light chain CDRs, the entire light chain variable region, some or all of the heavy chain CDRs, the entire heavy chain variable region, or a combination thereof of any of mouse anti-human Siglec-15 antibody 1H3 or 5G12.

The Siglec-15-binding molecules can include a CDR that is at least 45%, at least 50%, at least 55%, at least 60%, at least 65%, at least 70%, at least 75%, at least 80%, at least 85%, at least 90%, at least 95%, or at least 99% identical to the amino acid sequence of a CDR of the above-listed clones and which exhibit immunospecific binding to Siglec-15.

For example, the disclosed molecules can include one or more of the light chain CDR having the amino acid sequences of any of SEQ ID NO:4-6, 24-23, 30, 32, 34, and 36. The molecule can include at least one light chain CDR1, one light chain CDR2, and one light chain CDR3. For example, the molecule can include a light chain CDR1 including an amino acid sequence selected from the group consisting of SEQ ID NO: 4, 24, 30, 32, and 34. The molecule can include a light chain CDR2 including an amino acid sequence selected from the group consisting of SEQ ID NO: 5, 25, and 36. The molecule can include a light chain CDR3 including an amino acid sequence selected from the group consisting of SEQ ID NO: 6, and 26.

In particular embodiments, the molecule includes a light chain CDR1, a light chain CDR2, and a light chain CDR3 wherein the light chain CDR1, the light chain CDR2, and the light chain CDR3 include the amino acid sequences:

TABLE 1
Light Chain CDR Sequences

| | LCDR1 | LCDR2 | LCDR3 |
|---|---|---|---|
| | SEQ ID NO: | SEQ ID NO: | SEQ ID NO: |
| 1H3 | 4 | 5 | 6 |
| 5G12 | 24 | 25 | 26 |

The disclosed molecules can include one or more of the heavy chain CDR having the amino acid sequences of any of SEQ ID NO: 11, 15, 16, 17, 19, 21, 37, 41, 42, 45 and 46. The molecule can include at least one heavy chain CDR1, one heavy chain CDR2, and one heavy chain CDR3. The molecule can include a heavy chain CDR1 including an amino acid sequence selected from the group consisting of SEQ ID NO:12, 38, 43, and 47. The molecule can include a heavy chain CDR2 including an amino acid sequence selected from the group consisting of SEQ ID NO: 13, 18, 20, 22, 39, and 44. The molecule can include a heavy chain CDR3 including an amino acid sequence selected from the group consisting of SEQ ID NO: 14 and 40.

In particular embodiments, the molecule includes a heavy chain CDR1, a heavy chain CDR2, and a heavy chain CDR3 wherein the heavy chain CDR1, the heavy chain CDR2, and the heavy chain CDR3 include the amino acid sequences in Table 2.

TABLE 2
Heavy Chain CDR Sequences

| | HCDR1 | HCDR2 | HCDR3 |
|---|---|---|---|
| | SEQ ID NO: | SEQ ID NO: | SEQ ID NO: |
| 1H3 | 12 | 13 | 14 |
| 5G12 | 38 | 39 | 40 |

The Siglec-15-binding molecules can include an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, at least 99%, or 100% identical to the amino acid sequence of the variable heavy chain and/or light chain of the antibody produced by any of the above clones, and which exhibits immunospecific binding to human Siglec-15.

For example, the disclosed Siglec-15-binding molecules can include a light chain variable region having the amino acids sequence of SEQ ID NO: 3, 7, 8, 9, 10, 23, 27, 28, 29, 31, 33, and 35 or a variant thereof comprising at least 50%, 60%, 70%, 80%, 85%, 90%, 95%, 99%, or more sequence identity to SEQ ID NO: 3, 7, 8, 9, 10, 23, 27, 28, 29, 31, 33, and 35, and which exhibits immunospecifically binding to Siglec-15.

Additionally or alternatively the disclosed Siglec-15-binding molecules can include a heavy chain variable region having the amino acids sequence of SEQ ID NO: 11, 15, 16, 17, 19, 21, 37, 41, 42, 45 and 46, or a variant thereof comprising at least 50%, 60%, 70%, 80%, 85%, 90%, 95%, 99%, or more sequence identity to SEQ ID NO: 11, 15, 16, 17, 19, 21, 37, 41, 42, 45 and 46 and which exhibits immunospecifically binding to Siglec-15.

The Siglec-15-binding molecule can be an immunoglobulin molecule (e.g., an antibody, diabody, fusion protein, etc.) that includes one, two or three light chain CDRs and one, two or three heavy chain CDRs (e.g., in some embodiments, three light chain CDRs and three heavy chain CDRs), wherein the light chain CDRs include:
(1) the light chain CDR1 of murine anti-human Siglec-15 antibody 1H3 or 5G12, or a humanized variant thereof;
(2) the light chain CDR2 of murine anti-human Siglec-15 antibody 1H3 or 5G12, or a humanized variant thereof;
(3) the light chain CDR3 of murine anti-human Siglec-15 antibody 1H3 or 5G12, or a humanized variant thereof;
(4) the light chain CDR1 and the light chain CDR2 of mouse anti-human Siglec-15 antibody 1H3 or 5G12, or a humanized variant thereof;
(5) the light chain CDR1 and the light chain CDR3 of mouse anti-human Siglec-15 antibody 1H3 or 5G12, or a humanized variant thereof;
(6) the light chain CDR2 and the light chain CDR3 of mouse anti-human Siglec-15 antibody 1H3 or 5G12, or a humanized variant thereof; or
(7) the light chain CDR1, the light chain CDR2, and the light chain CDR3 mouse of anti-human Siglec-15 antibody 1H3 or 5G12, or humanized variant thereof.

The molecule can be an immunoglobulin molecule includes one, two or three light chain CDRs and one, two or three heavy chain CDRs (e.g., in some embodiments, three light chain CDRs and three heavy chain CDRs), wherein the heavy chain CDRs include:
(1) the heavy chain CDR1 of murine anti-human Siglec-15 antibody 1H3 or 5G12, or a humanized variant thereof;
(2) the heavy chain CDR2 of murine anti-human Siglec-15 antibody 1H3 or 5G12, or a humanized variant thereof;
(3) the heavy chain CDR3 of murine anti-human Siglec-15 antibody 1H3 or 5G12, or a humanized variant thereof;
(4) the heavy chain CDR1 and the heavy chain CDR2 of murine anti-human Siglec-15 antibody 1H3 or 5G12, or a humanized variant thereof;
(5) the heavy chain CDR1 and the heavy chain CDR3 of murine anti-human Siglec-15 antibody 1H3 or 5G12, or a humanized variant thereof;
(6) the heavy chain CDR2 and the heavy chain CDR3 of murine anti-human Siglec-15 antibody 1H3 or 5G12, or a humanized variant thereof; or
(7) the heavy chain CDR1, the heavy chain CDR2, and the heavy chain CDR3 murine anti-human Siglec-15 antibody 1H3 or 5G12, or a humanized variant thereof.

The molecule can be an immunoglobulin molecule that includes one, two or three light chain CDRs and one, two or three heavy chain CDRs (e.g., in some embodiments, three light chain CDRs and three heavy chain CDRs), wherein the light chain CDRs include:
(1) the light chain CDR1 of murine anti-human Siglec-15 antibody 1H3 or 5G12, or a humanized variant thereof;
(2) the light chain CDR2 of murine anti-human Siglec-15 antibody 1H3 or 5G12, or a humanized variant thereof;
(3) the light chain CDR3 of murine anti-human Siglec-15 antibody 1H3 or 5G12, or a humanized variant thereof;
(4) the light chain CDR1 and the light chain CDR2 of murine anti-human Siglec-15 antibody 1H3 or 5G12, or a humanized variant thereof;
(5) the light chain CDR1 and the light chain CDR3 of murine anti-human Siglec-15 antibody 1H3 or 5G12, or a humanized variant thereof;
(6) the light chain CDR2 and the light chain CDR3 of murine anti-human Siglec-15 antibody 1H3 or 5G12, or a humanized variant thereof; or
(7) the light chain CDR1, the light chain CDR2, and the light chain CDR3 of murine anti-human Siglec-15 antibody 1H3 or 5G12, or a humanized variant thereof,
and wherein the heavy chain CDRs include:

(1) the heavy chain CDR1 of murine anti-human Siglec-15 antibody 1H3 or 5G12, or a humanized variant thereof;
(2) the heavy chain CDR2 of murine anti-human Siglec-15 antibody 1H3 or 5G12, or a humanized variant thereof;
(3) the heavy chain CDR3 of murine anti-human Siglec-15 antibody 1H3 or 5G12, or a humanized variant thereof;
(4) the heavy chain CDR1 and the heavy chain CDR2 of murine anti-human Siglec-15 antibody 1H3 or 5G12, or a humanized variant thereof;
(5) the heavy chain CDR1 and the heavy chain CDR3 of murine anti-human Siglec-15 antibody 1H3 or 5G12, or a humanized variant thereof;
(6) the heavy chain CDR2 and the heavy chain CDR3 of murine anti-human Siglec-15 antibody 1H3 or 5G12, or a humanized variant thereof; or
(7) the heavy chain CDR1, the heavy chain CDR2, and the heavy chain CDR3 of murine anti-human Siglec-15 antibody 1H3 or 5G12, or a humanized variant thereof.

For example, the antibody can have one or more CDR of murine 1H3 or 5G12, or a chimeric antibody thereof, or a humanized variant having the CDR(s) corresponding to the CDR(s) of murine anti-human Siglec-15 antibody 1H3 or 5G12.

One embodiment provides a humanized monoclonal antibody having a variable light chain amino acid sequence that is at least 45%, at least 50%, at least 55%, at least 60%, at least 65%, at least 70%, at least 75%, at least 80%, at least 85%, at least 90%, at least 95%, at least 99%, or 100% identical to the amino acid sequence selected from the group consisting of SEQ ID NO: 4, 5, 6, 24, 25, 26, 30, 32, 34 and 36 and/or a variable heavy chain amino acid sequence that is at least 45%, at least 50%, at least 55%, at least 60%, at least 65%, at least 70%, at least 75%, at least 80%, at least 85%, at least 90%, at least 95%, at least 99%, or 100% identical to the amino acid sequence selected from the group consisting of SEQ ID NO: 12, 13, 14, 18, 20, 22, 43, 44, and 47.

### 3. Antibody Compositions

The disclosed Siglec-15-binding molecules can be antibodies or antigen binding fragments thereof. The disclosed antibodies and antigen binding fragments thereof include whole immunoglobulin (i.e., an intact antibody) of any class, fragments thereof, and synthetic proteins containing at least the antigen binding variable domain of an antibody. In some embodiments, the disclosed molecule contains both an antibody light chain as well as at least the variable domain of an antibody heavy chain. In other embodiments, such molecules can further include one or more of the CH1, hinge, CH2, CH3, and CH4 regions of the heavy chain (especially, the CH1 and hinge regions, or the CH1, hinge and CH2 regions, or the CH1, hinge, CH2 and CH3 regions). The antibody can be selected from any class of immunoglobulins, including IgM, IgG, IgD, IgA and IgE, and any isotype, including IgG₁, IgG₂, IgG₃ and IgG₄. In some embodiments, the constant domain is a complement fixing constant domain where it is desired that the antibody exhibit cytotoxic activity, and the class is typically IgG₁. In other embodiments, where such cytotoxic activity is not desirable, the constant domain can be of the IgG₂ or IgG₄ class. The antibody can include sequences from more than one class or isotype, and selecting particular constant domains to optimize desired effector functions is within the ordinary skill in the art.

The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic protein of the present disclosure. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

A monoclonal antibody is obtained from a substantially homogeneous population of antibodies, i.e., the individual antibodies within the population are identical except for possible naturally occurring mutations that may be present in a small subset of the antibody molecules. Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

a. Chimeric and Humanized Antibodies

Chimeric antibodies and antigen binding fragments thereof including one or more of the disclosed sequences and functional variants thereof are also provided.

Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397. Chimeric antibodies including one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, Proc. Natl. Acad. Sci. USA 91:969), and chain shuffling (U.S. Pat. No. 5,565,332).

The disclosed molecules can be human or humanized antibodies, or antigen binding fragments thereof. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)₂, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also contain residues that are found neither in the recipient antibody nor in the imported CDR or framework sequences. In general, the humanized antibody will contain substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will contain at least a portion of an immunoglobulin constant region (Fc), typically that of a human immunoglobulin.

Methods for humanizing non-human antibodies are well known in the art, see, for example, European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J. Immunol. 169:1119-1125; Caldas et al., 2000, Protein Eng. 13:353-360; Morea et al., 2000, Methods 20:267-79; Baca et al., 1997, J. Biol. Chem. 272:10678-10684; Roguska et al., 1996, Protein Eng. 9:895-904; Couto et al., 1995, Cancer Res. 55 (23 Supp):5973s-5977s; Couto et al., 1995, Cancer Res. 55:1717-22; Sandhu, 1994, Gene 150:409-10; Pedersen et al., 1994, J. Mol. Biol. 235:959-973; Jones et al., 1986, Nature 321:522-525; Reichmann et al., 1988, Nature 332:323-329; and Presta, 1992, Curr. Op. Struct. Biol. 2:593-596).

Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a non-human antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies can be very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

A human, humanized or chimeric antibody derivative can include substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Such antibodies can also include at least a portion of an immunoglobulin constant region (Fc), typically that of a human immunoglobulin. The constant domains of such antibodies can be selected with respect to the proposed function of the antibody, in particular the effector function which may be required. In some embodiments, the constant domains of such antibodies are or can include human IgA, IgD, IgE, IgG or IgM domains. In a specific embodiment, human IgG constant domains, especially of the IgG1 and IgG3 isotypes are used, when the humanized antibody derivative is intended for a therapeutic use and antibody effector functions such as antibody-dependent cell-mediated cytotoxicity (ADCC) and complement-dependent cytotoxicity (CDC) activity are needed. In alternative embodiments, IgG2 and IgG4 isotypes are used when the antibody is intended for therapeutic purposes and antibody effector function is not required. Fc constant domains including one or more amino acid modifications which alter antibody effector functions such as those disclosed in U.S. Patent Application Publication Nos. 2005/0037000 and 2005/0064514.

The framework and CDR regions of a humanized antibody need not correspond precisely to the parental sequences, e.g., the donor CDR or the consensus framework can be mutagenized by substitution, insertion or deletion of at least one residue so that the CDR or framework residue at that site does not correspond to either the consensus or the donor antibody. In some embodiments, such mutations not extensive. Usually, at least 75% of the humanized antibody residues will correspond to those of the parental framework region (FR) and CDR sequences, more often 90%, or greater than 95%. Humanized antibodies can be produced using variety of techniques known in the art, including, but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, Proc. Natl. Acad. Sci. 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp):5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596.

Often, framework residues in the framework regions will be substituted with the corresponding residue from the CDR donor antibody to alter, for example improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., 1988, Nature 332:323).

Human, chimeric or humanized derivatives of the disclosed murine anti-human Siglec-15 antibodies can be used for in vivo methods in humans. Murine antibodies or antibodies of other species can be advantageously employed for many uses (for example, in vitro or in situ detection assays, acute in vivo use, etc.). Such a human or humanized antibody can include amino acid residue substitutions, deletions or additions in one or more non-human CDRs. The humanized antibody derivative can have substantially the same binding, stronger binding or weaker binding when compared to a non-derivative humanized antibody. In specific embodiments, one, two, three, four, or five amino acid residues of the CDR have been substituted, deleted or added (i.e., mutated). Completely human antibodies are particularly desirable for therapeutic treatment of human subjects.

Such human antibodies can be made by a variety of methods known in the art including phage display methods using antibody libraries derived from human immunoglobulin sequences (see U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741). Such human antibodies can be produced using transgenic mice which are incapable of expressing functional endogenous immunoglobulins, but which can express human immunoglobulin genes.

For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen, e.g., all or a portion of a polypeptide. Monoclonal antibodies directed against the antigen can be obtained from the immunized, transgenic mice using conventional hybridoma technology (see, e.g., U.S. Pat. No. 5,916,771). The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93, which is incorporated herein by reference in its entirety). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, CA) and Medarex (Princeton, NJ) can be engaged to provide human antibodies directed against a selected antigen using technology similar to that described above.

DNA sequences coding for human acceptor framework sequences include but are not limited to FR segments from the human germline VH segment VH1-18 and JH6 and the human germline VL segment VK-A26 and JK4. In a specific embodiment, one or more of the CDRs are inserted within framework regions using routine recombinant DNA techniques. The framework regions can be naturally occurring or consensus framework regions, and human framework regions (see, e.g., Chothia et al., 1998, “Structural Determinants In The Sequences Of Immunoglobulin Variable Domain,” J. Mol. Biol. 278: 457-479 for a listing of human framework regions).

i. Humanized Antibodies

One embodiment provides a humanized 1H3 and 5G12 antibodies or antigen binding fragments thereof. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)₂, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also contain residues that are found neither in the recipient antibody nor in the imported CDR or framework sequences. In general, the humanized antibody will contain substantially all of at least one, and typically two, variable domains, in which all or substantially all, of the CDR regions correspond to those of a non-human immunoglobulin and all or substantially all of the FR regions are those of a human immunoglobulin consensus sequence. The humanized antibody optimally also will contain at least a portion of an immunoglobulin constant region (Fc), typically that of a human immunoglobulin.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a nonhuman antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

The antibody can be bound to a substrate or labeled with a detectable moiety or both bound and labeled. The detectable moieties contemplated with the present compositions include fluorescent, enzymatic and radioactive markers.

b. Single-Chain Antibodies

The Siglec-15-binding molecules can be single-chain antibodies. Methods for the production of single-chain antibodies are well known to those of skill in the art. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation. These Fvs lack the constant regions (Fc) present in the heavy and light chains of the native antibody.

c. Monovalent Antibodies

In vitro methods are also suitable for preparing monovalent antibodies. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment, called the F(ab′)₂ fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)₂ fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

d. Hybrid Antibodies

In some embodiments, the antibodies are hybrid antibodies. In hybrid antibodies, one heavy and light chain pair is homologous to that found in an antibody raised against one epitope, while the other heavy and light chain pair is homologous to a pair found in an antibody raised against another epitope. This results in the property of multi-functional valency, i.e., ability to bind at least two different epitopes simultaneously. Such hybrids can be formed by fusion of hybridomas producing the respective component antibodies, or by recombinant techniques. Such hybrids may, of course, also be formed using chimeric chains.

e. Conjugates or Fusions of Antibody Fragments

The targeting function of the antibody can be used therapeutically by coupling the antibody or a fragment thereof with a therapeutic agent. Such coupling of the antibody or fragment (e.g., at least a portion of an immunoglobulin constant region (Fc)) with the therapeutic agent can be achieved by making an immunoconjugate or by making a fusion protein, comprising the antibody or antibody fragment and the therapeutic agent.

Such coupling of the antibody or fragment with the therapeutic agent can be achieved by making an immunoconjugate or by making a fusion protein, or by linking the antibody or fragment to a nucleic acid, such as an siRNA, or a peptide or protein, comprising the antibody or antibody fragment and the therapeutic agent.

In some embodiments, the antibody is modified to alter its half-life. In some embodiments, it is desirable to increase the half-life of the antibody so that it is present in the circulation or at the site of treatment for longer periods of time. For example, it may be desirable to maintain titers of the antibody in the circulation or in the location to be treated for extended periods of time. Antibodies can be engineered with Fc variants that extend half-life, e.g., using Xtend™ antibody half-life prolongation technology (Xencor, Monrovia, CA). In other embodiments, the half-life of the anti-DNA antibody is decreased to reduce potential side effects. The conjugates disclosed can be used for modifying a given biological response. The drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin.

f. Mono and Multi-Specific Antibodies

In some embodiments the disclosed antibodies are monospecific, binding only to Siglec-15. Bispecific derivatives of such antibodies, trispecific derivatives of such antibodies or derivative antibodies of greater multi-specificity, that exhibit specificity to different immune system targets in addition to their specificity for human Siglec-15 are also provided. For example, such antibodies can bind to both human Siglec-15 and to an antigen that is important for targeting the antibody to a particular cell type or tissue (for example, to an antigen associated with a cancer antigen of a tumor being treated). In another embodiment, such multispecific antibody binds to molecules (receptors or ligands) involved in alternative immunomodulatory pathways, such as B7-H1, PD-1, CTLA4, TIM3, TIM4, OX40, CD40, GITR, 4-1-BB, LIGHT or LAG3, in order to enhance the immunomodulatory effects and combine multiple mechanisms of action, such as ligand blocking, immune cell activation and direct tumor targeting, in one molecule.

g. Derivatives

Production and use of “derivatives” of any of the disclosed Siglec-15-binding molecules are also disclosed. A derivative molecule, for example an antibody or antibody fragment, can be modified by chemical modifications using techniques known to those of skill in the art, including, but not limited to, specific chemical cleavage, acetylation, formulation, metabolic synthesis of tunicamycin, etc. The term derivative encompasses non amino acid modifications, for example, amino acids that can be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function.

In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1----6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740).

The disclosed antibodies can be modified by recombinant means to increase greater efficacy of the antibody in mediating the desired function. Thus, antibodies can be modified by substitutions using recombinant means. Typically, the substitutions will be conservative substitutions. For example, at least one amino acid in the constant region of the antibody can be replaced with a different residue. See, e.g., U.S. Pat. Nos. 5,624,821, 6,194,551, Application No. WO 9958572; and Angal, et al., Mol. Immunol. 30:105-08 (1993). The modification in amino acids includes deletions, additions, substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to Siglec-15 polypeptides, or fragments, or fusions thereof. See e.g., Antibody Engineering: A Practical Approach (Oxford University Press, 1996).

In some embodiments, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

Substitutions, additions or deletions in the derivatized antibodies can be in the Fc region of the antibody and can thereby serve to modify the binding affinity of the antibody to one or more FcγR. Methods for modifying antibodies with modified binding to one or more FcγR are known in the art, see, e.g., PCT Publication Nos. WO 04/029207, WO 04/029092, WO 04/028564, WO 99/58572, WO 99/51642, WO 98/23289, WO 89/07142, WO 88/07089, and U.S. Pat. Nos. 5,843,597 and 5,642,821.

In some embodiments, antibodies whose Fc region have been deleted (for example, a Fab or F(ab)2, etc.) or modified so that the molecule exhibits diminished or no Fc receptor (FcR) binding activity, or exhibits enhanced antibody-dependent cell-mediated cytotoxicity (ADCC) or complement dependent cytotoxicity (CDC) activities. In some embodiments, the antibodies have altered affinity for an activating FcγR, e.g., FcγRIIIA. Such modifications can also have an altered Fc-mediated effector function. Modifications that affect Fc-mediated effector function are well known in the art (see U.S. Pat. No. 6,194,551, and WO 00/42072). In one particular embodiment, the modification of the Fc region results in an antibody with an altered antibody-mediated effector function, an altered binding to other Fc receptors (e.g., Fc activation receptors), an altered antibody-dependent cell-mediated cytotoxicity (ADCC) activity, an altered C1q binding activity, an altered complement-dependent cytotoxicity activity (CDC), a phagocytic activity, or any combination thereof.

Derivatized antibodies can be used to alter the half-lives (e.g., serum half-lives) of parental antibodies in a mammal, such as a human. For example, such alteration can result in a half-life of greater than 15 days, greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the humanized antibodies or fragments thereof in a mammal, such as a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor. The humanized antibodies can be engineered to increase biological half-lives (see, e.g. U.S. Pat. No. 6,277,375). For example, humanized antibodies can be engineered in the Fc-hinge domain to have increased in vivo or serum half-lives.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The antibodies can also be modified by the methods and coupling agents described by Davis et al. (See U.S. Pat. No. 4,179,337) in order to provide compositions that can be injected into the mammalian circulatory system with substantially no immunogenic response.

The framework residues of the humanized antibodies can be modified. Residues in the framework regions can be substituted with the corresponding residue from the CDR donor antibody to alter, for example improve, antigen binding. These framework substitutions can be identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann, L. et al. (1988) “Reshaping Human Antibodies For Therapy,” Nature 332:323-327).

The disclosed Siglec-15-binding molecules can be recombinantly fused or chemically conjugated (including both covalently and non-covalently conjugations) to a heterologous molecule (i.e., an unrelated molecule). The fusion does not necessarily need to be direct, but may occur through linker sequences.

In some embodiments such heterologous molecules are polypeptides having at least 10, at least 20, at least 30, at least 40, at least 50, at least 60, at least 70, at least 80, at least 90 or at least 100 amino acids. Such heterologous molecules can alternatively be enzymes, hormones, cell surface receptors, drug moieties, such as: macrophage-specific targeting reagents (such as the intracellular carboxylesterase, hCE1 (Needham, L. A. et al. (2011) “Drug Targeting To Monocytes And Macrophages Using Esterase-Sensitive Chemical Motif,” J. Pharmacol. Exp. Ther. DOI:10.1124/jpet.111.183640), chitin and chitosan (Muzzarelli, R. A. (2010) “Chitins And Chitosans As Immunoadjuvants And Non-Allergenic Drug Carriers,” Mar Drugs 8(2):292-312), galactosylated low-density lipoprotein (Wu, F. et al. (009) “Galactosylated LDL Nanoparticles: A Novel Targeting Delivery System To Deliver Antigen To Macrophages And Enhance Antigen Specific T Cell Responses,” Molec. Pharm. 6(5):1506-1517), N-formyl-Met-Leu-Phe (fMLF), a macrophage-specific chemo-attractant (Wan, L. et al. (2008) “Optimizing Size And Copy Number For PEG-Fmlf (N-Formyl-Methionyl-Leucyl-Phenylalanine) Nanocarrier Uptake By Macrophages,” Bioconjug. Chem. 19(1):28-38), maleylated or mannosylated protein, such as maleylated albumin (Anatelli, F. et al. (2006) “Macrophage-Targeted Photosensitizer Conjugate Delivered By Intratumoral Injection,” Mol Pharm. 3(6):654-664; Bansal, P. et al. (1999) “MHC Class I-Restricted Presentation Of Maleylated Protein Binding To Scavenger Receptors,” J. Immunol. 162(8):4430-4437); see also Mukhopadhyay, A. et al. (2003) “Intracellular Delivery Of Drugs To Macrophages,” Adv. Biochem. Eng. Biotechnol. 84:183-209), toxins (such as abrin, ricin A, pseudomonas exotoxin (i.e., PE-40), diphtheria toxin, ricin, gelonin, or pokeweed antiviral protein), proteins (such as tumor necrosis factor, interferon (e.g., α-interferon, β-interferon), nerve growth factor, platelet derived growth factor, tissue plasminogen activator, or an apoptotic agent (e.g., tumor necrosis factor-α, tumor necrosis factor-β)), biological response modifiers (such as, for example, a lymphokine (e.g., interleukin-1 (“IL-1”), interleukin-2 (“IL-2”), interleukin-6 (“IL-6”)), granulocyte macrophage colony stimulating factor (“GM-CSF”), granulocyte colony stimulating factor (“G-CSF”), or macrophage colony stimulating factor, (“M-CSF”), or growth factors (e.g., growth hormone (“GH”))), cytotoxins (e.g., a cytostatic or cytocidal agent, such as paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof), antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, BiCNU® (carmustine; BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), or anti-mitotic agents (e.g., vincristine and vinblastine).

In another embodiment, the molecules are conjugated to a second antibody to form an antibody heteroconjugate as described by Segal in U.S. Pat. No. 4,676,980. Such heteroconjugate antibodies can additionally bind to haptens (such as fluorescein, etc.), or to cellular markers (e.g., 4-1-BB, B7-H1, PD-1, CD4, CD8, CD14, CD25, CD27, CD40, CD68, CD163, CTLA4, GITR, LAG-3, OX40, TIM3, TIM4, TLR2, LIGHT, etc.) or to cytokines (e.g., IL-4, IL-7, IL-10, IL-12, IL-15, IL-17, TGF-beta, IFNg, Flt3, BLys) or chemokines (e.g., CCL21), etc.

The Fc portion of the fusion protein can be varied by isotype or subclass, can be a chimeric or hybrid, and/or can be modified, for example to improve effector functions, control of half-life, tissue accessibility, augment biophysical characteristics such as stability, and improve efficiency of production (and less costly). Many modifications useful in construction of disclosed fusion proteins and methods for making them are known in the art, see for example Mueller, J. P. et al. (1997) “Humanized Porcine VCAM-Specific Monoclonal Antibodies With Chimeric IgG2/G4 Constant Regions Block Human Leukocyte Binding To Porcine Endothelial Cells,” Mol. Immun. 34(6):441-452, Swann, P. G. (2008) “Considerations For The Development Of Therapeutic Monoclonal Antibodies,” Curr. Opin. Immun. 20:493-499 (2008), and Presta, L. G. (2008) “Molecular Engineering And Design Of Therapeutic Antibodies,” Curr. Opin. Immun. 20:460-470. In some embodiments the Fc region is the native IgG1, IgG2, or IgG4 Fc region. In some embodiments the Fc region is a hybrid, for example a chimeric consisting of IgG2/IgG4 Fc constant regions. Modifications to the Fc region include, but are not limited to, IgG4 modified to prevent binding to Fc gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn. The Fc region can include the entire hinge region, or less than the entire hinge region.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. Therefore, the Fc domain can the disclosed antibodies and fragments contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA).

Another embodiment includes IgG2-4 hybrids and IgG4 mutants that have reduced binding to FcγR, which increases their half-life. Representative IgG2-4 hybrids and IgG4 mutants are described in Angal, S. et al. (1993) “A Single Amino Acid Substitution Abolishes The Heterogeneity Of Chimeric Mouse/Human (Igg4) Antibody,” Molec. Immunol. 30(1):105-108; Mueller, J. P. et al. (1997) “Humanized Porcine VCAM-Specific Monoclonal Antibodies With Chimeric IgG2/G4 Constant Regions Block Human Leukocyte Binding To Porcine Endothelial Cells,” Mol. Immun. 34(6):441-452; and U.S. Pat. No. 6,982,323. In some embodiments the IgG1 and/or IgG2 domain is modified; for example, Angal, S. et al. (1993) describe IgG1 and IgG2 variants in which serine 241 is replaced with proline.

In some embodiments, the Fc domain of such molecules contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890. Exemplary variants of human IgG1 Fc domains with reduced binding to CD32B and/or increased binding to CD16A contain F243L, R929P, Y300L, V305I or P296L substitutions. These amino acid substitutions can be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution. In another embodiment, the human IgG1 Fc domain variant contains an N297Q substitution, as this mutation abolishes FcR binding.

Techniques for conjugating therapeutic moieties to antibodies are well known; see, e.g., Arnon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al. (1982) “The Preparation And Cytotoxic Properties Of Antibody-Toxin Conjugates,” Immunol. Rev. 62:119-158.

Any of the disclosed molecules can be fused to marker sequences, such as a peptide, to facilitate purification. In some embodiments, the marker amino acid sequence is a hexa-histidine peptide, the hemagglutinin “HA” tag, which corresponds to an epitope derived from the influenza hemagglutinin protein (Wilson, I. A. et al. (1984) “The Structure Of An Antigenic Determinant In A Protein,” Cell, 37:767-778) and the “flag” tag (Knappik, A. et al. (1994) “An Improved Affinity Tag Based On The FLAG Peptide For The Detection And Purification Of Recombinant Antibody Fragments,” Biotechniques 17(4):754-761).

The disclosed Siglec-15-binding molecules can be conjugated to a diagnostic or therapeutic agent, or another molecule for which serum half-life is desired to be increased. The antibodies can be used diagnostically (in vivo, in situ or in vitro) to, for example, monitor the development or progression of a disease, disorder or infection as part of a clinical testing procedure to, e.g., determine the efficacy of a given treatment regimen or to select patients more likely to respond to a particular therapy (such as those expressing high levels of Siglec-15).

Detection can be facilitated by coupling the molecule, such as antibody or an antigen binding fragment thereof, to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance can be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹¹³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (3H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The disclosed molecules can be attached to solid supports, which are particularly useful for immunoassays or purification of the target antigen or of other molecules that are capable of binding to target antigen that has been immobilized to the support via binding to an antibody or antigen-binding fragment. Such solid supports include, but are not limited to, glass, cellulose, polyacrylamide, nylon, polystyrene, polyvinyl chloride or polypropylene.

Nucleic acid molecules (DNA or RNA) that encode any such antibodies, fusion proteins or fragments, as well as vector molecules (such as plasmids) that are capable of transmitting or of replicating such nucleic acid molecules are also disclosed. The nucleic acids can be single-stranded, double-stranded, can contain both single-stranded and double-stranded portions.

### 4. Method of Making

The Siglec-15-binding molecules can be produced by any method known in the art useful for the production of polypeptides, e.g., in vitro synthesis, recombinant DNA production, and the like. The humanized antibodies are typically produced by recombinant DNA technology. The antibodies can be produced using recombinant immunoglobulin expression technology. The recombinant production of immunoglobulin molecules, including humanized antibodies are described in U.S. Pat. No. 4,816,397 (Boss et al.), U.S. Pat. Nos. 6,331,415 and 4,816,567 (both to Cabilly et al.), U.K. patent GB 2,188,638 (Winter et al.), and U.K. patent GB 2,209,757. Techniques for the recombinant expression of immunoglobulins, including humanized immunoglobulins, can also be found, in Goeddel et al., Gene Expression Technology Methods in Enzymology Vol. 185 Academic Press (1991), and Borreback, Antibody Engineering, W. H. Freeman (1992). Additional information concerning the generation, design and expression of recombinant antibodies can be found in Mayforth, Designing Antibodies, Academic Press, San Diego (1993).

An exemplary process for the production of the recombinant chimeric antibodies can include the following: a) constructing, by conventional molecular biology methods, an expression vector that encodes and expresses an antibody heavy chain in which the CDRs and variable region of an anti-Siglec-15 antibody are fused to an Fc region derived from a human immunoglobulin, thereby producing a vector for the expression of a chimeric antibody heavy chain; b) constructing, by conventional molecular biology methods, an expression vector that encodes and expresses an antibody light chain of the murine anti-human Siglec-15 monoclonal antibody, thereby producing a vector for the expression of chimeric antibody light chain; c) transferring the expression vectors to a host cell by conventional molecular biology methods to produce a transfected host cell for the expression of chimeric antibodies; and d) culturing the transfected cell by conventional cell culture techniques so as to produce chimeric antibodies.

An exemplary process for the production of the recombinant humanized antibodies can include the following: a) constructing, by conventional molecular biology methods, an expression vector that encodes and expresses an anti-human Siglec-15 heavy chain in which the CDRs and a minimal portion of the variable region framework that are required to retain donor antibody binding specificity are derived from the humanized variants of anti-human Siglec-15 antibody(ies), and the remainder of the antibody is derived from a human immunoglobulin, thereby producing a vector for the expression of a humanized antibody heavy chain; b) constructing, by conventional molecular biology methods, an expression vector that encodes and expresses an antibody light chain in which the CDRs and a minimal portion of the variable region framework that are required to retain donor antibody binding specificity are derived from a non-human immunoglobulin, such as the disclosed murine anti-human Siglec-15 antibodies, and the remainder of the antibody is derived from a human immunoglobulin, thereby producing a vector for the expression of humanized antibody light chain; c) transferring the expression vectors to a host cell by conventional molecular biology methods to produce a transfected host cell for the expression of humanized antibodies; and d) culturing the transfected cell by conventional cell culture techniques so as to produce humanized antibodies.

With respect to either exemplary method, host cells can be co-transfected with such expression vectors, which can contain different selectable markers but, with the exception of the heavy and light chain coding sequences, can be identical. This procedure provides for equal expression of heavy and light chain polypeptides. Alternatively, a single vector can be used which encodes both heavy and light chain polypeptides. The coding sequences for the heavy and light chains can include cDNA or genomic DNA or both. The host cell used to express the recombinant antibody can be either a bacterial cell such as Escherichia coli, or a eukaryotic cell (e.g., a Chinese hamster ovary (CHO) cell or a HEK-293 cell). The choice of expression vector is dependent upon the choice of host cell, and can be selected so as to have the desired expression and regulatory characteristics in the selected host cell. Other cell lines that can be used include, but are not limited to, CHO-K1, NSO, and PER.C6 (Crucell, Leiden, Netherlands).

Any of the disclosed antibodies can be used to generate anti-idiotype antibodies using techniques well known to those skilled in the art (see, e.g., Greenspan, N. S. et al. (1989) “Idiotypes: Structure And Immunogenicity,” FASEB J. 7:437-444; and Nisinoff, A. (1991) “Idiotypes: Concepts And Applications,” J. Immunol. 147(8):2429-2438).

### E. Siglec-15 Ligand-Binding Molecules

Molecules that bind to Siglec-15 ligands, such as Siglec-15 proteins, Siglec-15 fusion proteins, and fragments and variants thereof are also provided. The Siglec-15 ligand-binding molecule can bind to Siglec-15 ligand such as a sialylated glycoprotein, LRRC4C, a Siglec-15-counter receptor, etc. In some embodiments, the Siglec-15 ligand-binding molecule can induce signal transduction through the Siglec-15 ligand. In some embodiments, the Siglec-15 ligand-binding molecule blocks or otherwise reduces interaction between Siglec-15 and its ligand, without inducing signal transduction through Siglec-15 or its ligand. Siglec-15 ligand-binding molecules can be used to modulate Siglec-15 activity as discussed in more detail below and illustrated in the Examples, and can be used to therapeutically to treat a subject in need thereof.

### 1. Siglec-15 Polypeptides

In some embodiments, the Siglec-15 ligand-binding molecule is Siglec-15, or fragment or variant thereof. For example, in some embodiments, the Siglec-15 ligand-binding molecules includes a polypeptide at least 95, 96, 97, 98, 99, or 100% identical to SEQ ID NO:1 or 2, or a fragment thereof such as the extracellular domain, or a subdomain thereof such as the IgV domain, the IgC domain or the combination thereof. In some embodiments, the Siglec-15 polypeptide is soluble or otherwise cell-free. For example, in some embodiments, the Siglec-15 lacks one or more of the transmembrane domain, the cytoplasmic domain, or the leader sequence.

### 2. Siglec-15 Fusion Proteins

In some embodiments, the Siglec-15 ligand-binding molecule is a Siglec-15 fusion protein. Fusion proteins containing Siglec-15 polypeptides coupled to other polypeptides to form fusion proteins are provided. Siglec-15 fusion polypeptides can have a first fusion partner comprising all or a part of a Siglec-15 protein fused (i) directly to a second polypeptide or, (ii) optionally, fused to a linker peptide sequence that is fused to the second polypeptide. The fusion proteins optionally contain a domain that functions to dimerize or multimerize two or more fusion proteins. In some embodiments the fusion protein is not or does not dimerize or multimerize. The peptide/polypeptide linker domain can either be a separate domain, or alternatively can be contained within one of one of the other domains (Siglec-15 polypeptide or second polypeptide) of the fusion protein. Similarly, the domain that functions to dimerize or multimerize the fusion proteins can either be a separate domain, or alternatively can be contained within one of one of the other domains (Siglec-15 polypeptide, second polypeptide or peptide/polypeptide linker domain) of the fusion protein. In some embodiments, the dimerization/multimerization domain and the peptide/polypeptide linker domain are the same.

Fusion proteins disclosed herein are of formula I:

N—R₁—R₂—R₃—C

wherein “N” represents the N-terminus of the fusion protein, “C” represents the C-terminus of the fusion protein, “R₁” is a Siglec-15 polypeptide, “R₂” is an optional peptide/polypeptide linker domain, and “R₃” is a second polypeptide. Alternatively, R₃ may be the Siglec-15 polypeptide and R₁ may be the second polypeptide.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric. As discussed above, in some embodiments the fusion protein is not or does not dimerize or multimerize.

In some embodiments, the second polypeptide contains one or more domains of an immunoglobulin heavy chain constant region, for example an amino acid sequence corresponding to the hinge, C_{H}2 and/or C_{H}3 regions of a human immunoglobulin Cγ1 chain, the hinge, C_{H}2 and/or C_{H}3 regions of a murine immunoglobulin Cγ2a chain, C_{H}2 and/or C_{H}3 regions of a human immunoglobulin Cγ1, ect.

The Fc portion of the fusion protein may be varied by isotype or subclass, may be a chimeric or hybrid, and/or may be modified, for example to improve effector functions, control of half-life, tissue accessibility, augment biophysical characteristics such as stability, and improve efficiency of production (and less costly). Many modifications useful in construction of disclosed fusion proteins and methods for making them are known in the art, see for example Mueller, et al., Mol. Immun., 34(6):441-452 (1997), Swann, et al., Cur. Opin. Immun., 20:493-499 (2008), and Presta, Cur. Opin. Immun. 20:460-470 (2008). In some embodiments the Fc region is the native IgG1, IgG2, or IgG4 Fc region. In some embodiments the Fc region is a hybrid, for example a chimeric consisting of IgG2/IgG4 Fc constant regions. Modifications to the Fc region include, but are not limited to, IgG4 modified to prevent binding to Fc gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn. The Fc region may include the entire hinge region, or less than the entire hinge region.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA).

Another embodiment includes IgG2-4 hybrids and IgG4 mutants that have reduced binding to FcR which increase their half-life. Representative IG2-4 hybrids and IgG4 mutants are described in Angal, S. et al., Molecular Immunology, 30(1):105-108 (1993); Mueller, J. et al., Molecular Immonology, 34(6): 441-452 (1997); and U.S. Pat. No. 6,982,323 to Wang et al. In some embodiments the IgG1 and/or IgG2 domain is deleted for example, Angal et al. describe IgG1 and IgG2 having serine 241 replaced with a proline.

In some embodiments, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, et al., Cancer Res., 57(18):8882-90 (2007). Exemplary variants of human IgG1 Fc domains with reduced binding to CD32B and/or increased binding to CD16A contain F243L, R929P, Y300L, V305I or P296L substitutions. These amino acid substitutions may be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution. In another embodiment, the human IgG1 Fc domain variant contains an N297Q substitution, as this mutation abolishes FcR binding.

The disclosed fusion proteins optionally contain a peptide or polypeptide linker domain that separates the Siglec-15 polypeptide from the second polypeptide. In some embodiments, the linker domain contains the hinge region of an immunoglobulin. In a preferred embodiment, the hinge region is derived from a human immunoglobulin. Suitable human immunoglobulins that the hinge can be derived from include IgG, IgD and IgA. In a preferred embodiment, the hinge region is derived from human IgG. Amino acid sequences of immunoglobulin hinge regions and other domains are well known in the art.

a. Siglec15 ECD-IgG1 Fc Fusion Protein

An exemplary fusion protein is Siglec15 ECD-IgG1 Fc Fusion Protein (L234F/L235E/P331S).

(SEQ ID NO: 49)
MEWSWVFLFFLSVTTGVHSFVRTKIDTTENLLNTEVHSSPAQRWSMQVPP
EVSAEAGDAAVLPCTFTHPHRHYDGPLTAIWRAGEPYAGPQVFRCAAARG
SELCQTALSLHGRFRLLGNPRRNDLSLRVERLALADDRRYFCRVEFAGDV
HDRYESRHGVRLHVTAAPRIVNISVLPSPAHAFRALCTAEGEPPPALAWS
GPALGNSLAAVRSPREGHGHLVTAELPALTHDGRYTCTAANSLGRSEASV
YLFRFHGASGDKTHTCPPCPAPE [symbol] GGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPA [symbol] IEKTISKAKGQPREPQVYTLPPSRDELTK
NQVSLTCLVKGFYPSDIAVEWESNGQPENNYKTTPPVLDSDGSFFLYSKL
TVDKSRWQQGNVFSCSVMHEALHNHYTQKSLSLSPG.

A Murine Leader Sequence is illustrated in underlined. A Siglec-15 extracellular domain (ECD) is in italics. A Hinge Region is double underlined. The remaining sequence is derived from IgG1 Fc. L234F/L235E/P331S mutations in the IgG1 Fc domain bolded and dotted-underlined.

In some embodiments, the leader sequence is cleaved or otherwise missing from fusion protein. For example, the fusion protein can have the sequence:

(SEQ ID NO: 50)
FVRTKIDTTENLLNTEVHSSPAQRWSMQVPPEVSAEAGDAAVLPCTFTHP
HRHYDGPLTAIWRAGEPYAGPQVFRCAAARGSELCQTALSLHGRFRLLGN
PRRNDLSLRVERLALADDRRYFCRVEFAGDVHDRYESRHGVRLHVTAAPR
IVNISVLPSPAHAFRALCTAEGEPPPALAWSGPALGNSLAAVRSPREGHG
HLVTAELPALTHDGRYTCTAANSLGRSEASVYLFRFHGASGDKTHTCPPC
PAPE [symbol] GGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
A [symbol] IEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPG.

In some embodiments, the fusion protein is at least 50, 60, 70, 80, 85, 90, 95, 96, 97, 98, 99, or 100% identical to SEQ ID NO:49 or 50.

In some embodiments, the leader sequence, the linker (e.g., the hinge region), the second fusion partner (e.g., IgG1 Fc domain), or a combination thereof are substitute for another sequence(s) (e.g., an alternative leader sequence, hinge, Fc domain, etc.). Suitable substitutes are well known in the art. See, for example, U.S. Pat. No. 9,005,616, which is specifically incorporated by reference in its entirety.

b. Siglec-15 Fc (IgG1 Fc; FES)

One embodiment provides a fusion protein having 50, 60, 70, 80, 85, 90, 95, 96, 97, 98, 99, or 100% sequence identity to Siglec-15—FES (hIgG1 FES mutation silenced Fc receptor binding) having the following sequence:

(SEQ ID NO: 51)
MEWSWVFLFFLSVTTGVHSFVRTKIDTTENLLNTEVHSSPAQRWSMQVPP
EVSAEAGDAAVLPCTFTHPHRHYDGPLTAIWRAGEPYAGPQVFRCAAARG
SELCQTALSLHGRFRLLGNPRRNDLSLRVERLALADDRRYFCRVEFAGDV
HDRYESRHGVRLHVTAAPRIVNISVLPSPAHAFRALCTAEGEPPPALAWS
GPALGNSLAAVRSPREGHGHLVTAELPALTHDGRYTCTAANSLGRSEASV
YLFRFHGASGDKTHTCPPCPDKTHTCPPCPAPEFEGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPASIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG

The underlined sequence is the leader sequence. The bolded and italicized sequence is the Siglec-15 extracellular domain (ECD). The double underlined sequence is a linker. The unmarked sequence is Fc Domain (IgG1 FES).

Another embodiment provides a fusion protein having 85%, 90%, 95%, or 100% sequence identity to Siglec-15 Fc (IgG1 Fc; FES) having the following sequence:

(SEQ ID NO: 52)
FVRTKIDTTENLLNTEVHSSPAQRWSMQVPPEVSAEAGDAAVLPCTFTHP
HRHYDGPLTAIWRAGEPYAGPQVFRCAAARGSELCQTALSLHGRFRLLGN
PRRNDLSLRVERLALADDRRYFCRVEFAGDVHDRYESRHGVRLHVTAAPR
IVNISVLPSPAHAFRALCTAEGEPPPALAWSGPALGNSLAAVRSPREGHG
HLVTAELPALTHDGRYTCTAANSLGRSEASVYLFRFHGASGDKTHTCPPC
PDKTHTCPPCPAPEFEGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPASIEKTISKAKGQPREPQVYTLPPSRDELTKNQVSLTCLV
KGFYPSDIAVEWESNGQPENNYKTTPPVLDSDGSFFLYSKLTVDKSRWQQ
GNVFSCSVMHEALHNHYTQKSLSLSPG

The bolded and italicized sequence is the Siglec-15 extracellular domain (ECD). The double underlined sequence is a linker. The unmarked sequence is Fc Domain (IgG1 FES).

c. Siglec-15 Fc (IgG4 Fc; G4P)

Another embodiment provides a fusion protein having 85%, 90%, 95%, or 100% sequence identity to Siglec-15 Fc (IgG4 Fc; G4P) having the following sequence:

(SEQ ID NO: 53)
MEWSWVFLFFLSVTTGVHSFVRTKIDTTENLLNTEVHSSPAQRWSMQVPP
EVSAEAGDAAVLPCTFTHPHRHYDGPLTAIWRAGEPYAGPQVFRCAAARG
SELCQTALSLHGRFRLLGNPRRNDLSLRVERLALADDRRYFCRVEFAGDV
HDRYESRHGVRLHVTAAPRIVNISVLPSPAHAFRALCTAEGEPPPALAWS
GPALGNSLAAVRSPREGHGHLVTAELPALTHDGRYTCTAANSLGRSEASV
YLFRFHGASGESKYGPPCPPCPAPEFLGGPSVFLFPPKPKDTLMISRTPE
VTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYS
RLTVDKSRWQEGNVESCSVMHEALHNHYTQKSLSLSLG*.

The underlined sequence is the leader sequence. The bolded and italicized sequence is the Siglec-15 extracellular domain (ECD). The double underlined sequence is the hG4P Fc (G4P sequence).

Another embodiment provides a fusion protein having 85%, 90%, 95%, or 100% sequence identity to Siglec-15 Fc (IgG4 Fc; G4P) having the following sequence:

(SEQ ID NO: 54)
FVRTKIDTTENLLNTEVHSSPAQRWSMQVPPEVSAEAGDAAVLPCTFTHP
HRHYDGPLTAIWRAGEPYAGPQVFRCAAARGSELCQTALSLHGRFRLLGN
PRRNDLSLRVERLALADDRRYFCRVEFAGDVHDRYESRHGVRLHVTAAPR
IVNISVLPSPAHAFRALCTAEGEPPPALAWSGPALGNSLAAVRSPREGHG
HLVTAELPALTHDGRYTCTAANSLGRSEASVYLFRFHGASGESKYGPPCP
PCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVESCSV
MHEALHNHYTQKSLSLSLG*.

The bolded and italicized sequence is the Siglec-15 extracellular domain (ECD). The double underlined sequence is the hG4P Fc (G4P sequence).

### 3. 1H3 Light Chain Fusion Proteins

### a. 1H3 VL-Kappa Constant Domain

One embodiment provides a 1H3 light chain variable domain-IgG1 Fc fusion protein having a sequence of

(SEQ ID NO: 55)
DIQMTQSPSSLSASVGDRVTITCKASDHINNWLAWYQQKPGKAPKLLISG
ATSLETGVPSRFSGSGSGTDYTFTISSLQPEDIATYYCQQYWSSPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC*.

The 1H3 light chain variable domain is in italics. The remaining sequence is derived from IgG1 kappa constant domain.

d. 1H3 hVL1-Kappa Constant Domain

One embodiment provides a humanized anti-1H3 hVL1-kappa constant domain having an amino acid sequence of

(SEQ ID NO: 56)
DIQMTQSPSSLSASVGDRVTITCKASDHINNWLAWYQQKPGKAPKLLISG
ATSLETGVPSRFSGSGSGTDYTFTISSLQPEDIATYYCQQYWSSPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC*

The 1H3 hVL1 light chain variable domain is in italics. The remaining sequence is derived from IgG1 kappa constant domain.

g. 1H3 hVL2-Kappa Constant Domain

One embodiment provides a humanized anti-1H3 hVL2-kappa constant domain antibody having an amino acid sequence of

(SEQ ID NO: 57)
DIQMTQSPSSLSASVGDRVTITCKASDHINNWLAWYQQKPGKVPKLLISG
ATSLETGVPSRFSGSGSGTDYTLTISSLQPEDVATYYCQQYWSSPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC*.

The humanized 1H3 hVL2 light chain variable domain is in italics. The remaining sequence is derived from IgG1 kappa constant domain.

j. 1H3 hVL3-Kappa Constant Domain

One embodiment provides a humanized anti-1H3 hVL3-kappa constant domain antibody having an amino acid sequence of

(SEQ ID NO: 58)
DIQMTQSPSSLSASVGDRVTITCKASDHINNWLAWYQQKPGKAPKLLISG
ATSLETGVPSRFSGSGSGTDYTLTISSLQPEDVATYYCQQYWSSPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC*.

The humanized 1H3 hVL3 light chain variable domain is in italics. The remaining sequence is derived from IgG1 kappa constant domain.

### 4. 1H3 Heavy Chain Fusion Proteins

### a. 1H3 VH-IgG1 Fc

One embodiment provides a 1H3 heavy chain variable domain-IgG1 Fc fusion protein having a sequence of

(SEQ ID NO: 59)
QVQLKESGPGLVAPSQSLSITCTVSGFSLSNYGVHWVRQPPGKGLEWLVL
IWSDGSTTYNSALKSRLSISKDNSKSQVFLKMNSLQTGDTAMYYCARHPY
DDYSGYYYTMDYWGQGTSVTVSSDKTHTCPPCPAPE [symbol] GGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPA [symbol] IEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG.

The 1H3 heavy chain variable domain is in italics. A hinge region is double underlined. The remaining sequence is derived from IgG1 Fc. L234F/L235E/P331S mutations in the IgG1 Fc domain bolded and dotted-underlined.

### b. 1H3 VH-Fc (IgG1 Fc; FES)

One embodiment provides a fusion protein having 50, 60, 70, 80, 85, 90, 95, 96, 97, 98, 99, or 100% sequence identity to 1H3 heavy chain variable domain Fc (IgG1 Fc; FES) having the following sequence:

(SEQ ID NO: 60)
QVQLKESGPGLVAPSQSLSITCTVSGFSLSNYGVHWVRQPPGKGLEWLVL
IWSDGSTTYNSALKSRLSISKDNSKSQVFLKMNSLQTGDTAMYYCARHPY
DDYSGYYYTMDYWGQGTSVTVSSDKTHTCPPCPDKTHTCPPCPAPEFEGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYT
QKSLSLSPG.

The bolded and italicized sequence is the 1H3 heavy chain variable domain. The double underlined sequence is a linker. The unmarked sequence is Fc Domain (IgG1 FES).

### c. 1H3 VH-Fc (IgG4 Fc; G4P)

Another embodiment provides a fusion protein having 85%, 90%, 95%, or 100% sequence identity to 1H3 LC-Fc (IgG4 Fc; G4P) having the following sequence:

(SEQ ID NO: 61)
QVQLKESGPGLVAPSQSLSITCTVSGFSLSNYGVHWVRQPPGKGLEWLVL
IWSDGSTTYNSALKSRLSISKDNSKSQVFLKMNSLQTGDTAMYYCARHPY
DDYSGYYYTMDYWGQGTSVTVSSESKYGPPCPPCPAPEFLGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSL
G*.

The bolded and italicized sequence is the 1H3 heavy variable domain. The double underlined sequence is the hG4P Fc (G4P sequence).

d. 1H3 hVH1-IgG1 Fc

One embodiment provides a humanized 1H3 hVH1 heavy chain variable domain-IgG1 Fc fusion protein having a sequence of

(SEQ ID NO: 62)
QVQLQESGPGLVKPSETLSLTCTVSGFSLSNYGVHWVRQPPGKGLEWIVL
IWSDGSTTYNSALKSRVTISKDTSKNQVSLKLSSVTAADTAVYYCARHPY
DDYSGYYYTMDYWGQGTLVTVSSDKTHTCPPCPAPE [symbol] GGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPA [symbol] IEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG.

The 1H3 hVH1 heavy chain variable domain is in italics. A hinge region is double underlined. The remaining sequence is derived from IgG1 Fc. L234F/L235E/P331S mutations in the IgG1 Fc domain bolded and dotted-underlined.

### e. 1H3 VH1-Fc (IgG1 Fc; FES)

One embodiment provides a fusion protein having 50, 60, 70, 80, 85, 90, 95, 96, 97, 98, 99, or 100% sequence identity to 1H3 hVH1 heavy chain variable domain Fc (IgG1 Fc; FES) having the following sequence:

(SEQ ID NO: 63)
QVQLQESGPGLVKPSETLSLTCTVSGFSLSNYGVHWVRQPPGKGLEWIVL
IWSDGSTTYNSALKSRVTISKDTSKNQVSLKLSSVTAADTAVYYCARHPY
DDYSGYYYTMDYWGQGTLVTVSSDKTHTCPPCPDKTHTCPPCPAPEFEGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPASIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYT
QKSLSLSPG

The bolded and italicized sequence is the 1H3 hVH1 heavy chain variable domain. The double underlined sequence is a linker. The unmarked sequence is Fc Domain (IgG1 FES).

### f. 1H3 VH1-Fc (IgG4 Fc; G4P)

Another embodiment provides a fusion protein having 85%, 90%, 95%, or 100% sequence identity to 1H3 hVH1-Fc (IgG4 Fc; G4P) having the following sequence:

(SEQ ID NO: 64)
QVQLQESGPGLVKPSETLSLTCTVSGFSLSNYGVHWVRQPPGKGLEWIVL
IWSDGSTTYNSALKSRVTISKDTSKNQVSLKLSSVTAADTAVYYCARHPY
DDYSGYYYTMDYWGQGTLVTVSSESKYGPPCPPCPAPEFLGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFFLYSRLTVDKSRWQEGNVESCSVMHEALHNHYTQKSLSLSL
G*.

The bolded and italicized sequence is the 1H3 hVH1 heavy variable domain. The double underlined sequence is the hG4P Fc (G4P sequence).

g. 1H3 hVH2-IgG1 Fc

One embodiment provides a humanized 1H3 hVH2 heavy chain variable domain-IgG1 Fc fusion protein having a sequence of

(SEQ ID NO: 65)
QVQLQESGPGLVKPSETLSLTCTVSGFSLSNYGVHWVRQPPGKGLEWIGL
IWSDGSTTYASALKSRVTISKDTSKNQVSLKLSSVTAADTAVYYCARHPY
DDYSGYYYTMDYWGQGTLVTVSDKTHTCPPCPAPE [symbol] GGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPA [symbol] IEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG.

The 1H3 hVH2 heavy chain variable domain is in italics. A hinge region is double underlined. The remaining sequence is derived from IgG1 Fc. L234F/L235E/P331S mutations in the IgG1 Fc domain bolded and dotted-underlined.

h. 1H3 hVH2-Fc (IgG1 Fc; FES)

One embodiment provides a fusion protein having 50, 60, 70, 80, 85, 90, 95, 96, 97, 98, 99, or 100% sequence identity to 1H3 hVH2 heavy chain variable domain Fc (IgG1 Fc; FES) having the following sequence:

(SEQ ID NO: 66)
QVQLQESGPGLVKPSETLSLTCTVSGFSLSNYGVHWVRQPPGKGLEWIGL
IWSDGSTTYASALKSRVTISKDTSKNQVSLKLSSVTAADTAVYYCARHPY
DDYSGYYYTMDYWGQGTLVTVSDKTHTCPPCPDKTHTCPPCPAPEFEGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPASIEKTISK
AKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQ
KSLSLSPG

The bolded and italicized sequence is the 1H3 hVH2 heavy chain variable domain. The double underlined sequence is a linker. The unmarked sequence is Fc Domain (IgG1 FES).

i. 1H3 hVH2-Fc (IgG4 Fc; G4P)

Another embodiment provides a fusion protein having 85%, 90%, 95%, or 100% sequence identity to 1H3 hVH2-Fc (IgG4 Fc; G4P) having the following sequence:

(SEQ ID NO: 67)
QVQLQESGPGLVKPSETLSLTCTVSGFSLSNYGVHWVRQPPGKGLEWIGL
IWSDGSTTYASALKSRVTISKDTSKNQVSLKLSSVTAADTAVYYCARHPY
DDYSGYYYTMDYWGQGTLVTVSESKYGPPCPPCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSLG
*.

The bolded and italicized sequence is the 1H3 hVH2 heavy variable domain. The double underlined sequence is the hG4P Fc (G4P sequence).

j. 1H3 hVH3-IgG1 Fc

One embodiment provides a humanized 1H3 hVH3 heavy chain variable domain-IgG1 Fc fusion protein having a sequence of

(SEQ ID NO: 68)
QVQLQESGPGLVKPSETLSLTCTVSGFSLSNYGVHWVRQPPGKGLEWIGL
IWSDGSTTYNPSLKSRVTISKDTSKNQVSLKLSSVTAADTAVYYCARHPY
DDYSGYYYTMDYWGQGTLVTVSDKTHTCPPCPAPE [symbol] GGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPA [symbol] IEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG.

The 1H3 hVH3 heavy chain variable domain is in italics. A hinge region is double underlined. The remaining sequence is derived from IgG1 Fc. L234F/L235E/P331S mutations in the IgG1 Fc domain bolded and dotted-underlined.

k. 1H3 hVH3-Fc (IgG1 Fc; FES)

One embodiment provides a fusion protein having 50, 60, 70, 80, 85, 90, 95, 96, 97, 98, 99, or 100% sequence identity to 1H3 hVH3 heavy chain variable domain Fc (IgG1 Fc; FES) having the following sequence:

(SEQ ID NO: 69)
QVQLQESGPGLVKPSETLSLTCTVSGFSLSNYGVHWVRQPPGKGLEWIGL
IWSDGSTTYNPSLKSRVTISKDTSKNQVSLKLSSVTAADTAVYYCARHPY
DDYSGYYYTMDYWGQGTLVTVSDKTHTCPPCPDKTHTCPPCPAPEFEGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPASIEKTISK
AKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQ
KSLSLSPG

The bolded and italicized sequence is the 1H3 hVH3 heavy chain variable domain. The double underlined sequence is a linker. The unmarked sequence is Fc Domain (IgG1 FES).

l 1H3 hVH3-Fc (IgG4 Fc; G4P)

Another embodiment provides a fusion protein having 85%, 90%, 95%, or 100% sequence identity to 1H3 hVH3-Fc (IgG4 Fc; G4P) having the following sequence:

(SEQ ID NO:70)
QVQLQESGPGLVKPSETLSLTCTVSGFSLSNYGVHWVRQPPGKGLEWIGL
IWSDGSTTYNPSLKSRVTISKDTSKNQVSLKLSSVTAADTAVYYCARHPY
DDYSGYYYTMDYWGQGTLVTVSESKYGPPCPPCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSLG
*.

The bolded and italicized sequence is the 1H3 hVH3 heavy variable domain. The double underlined sequence is the hG4P Fc (G4P sequence).

m. 1H3 hVH4-IgG1 Fc

One embodiment provides a humanized 1H3 hVH4 heavy chain variable domain-IgG1 Fc fusion protein having a sequence of

(SEQ ID NO: 71)
QVQLQESGPGLVKPSETLSLTCTVSGFSLSNYGVHWVRQPPGKGLEWIGL
IWSEGSTTYASALKSRVTISKDTSKNQVSLKLSSVTAADTAVYYCARHPY
DDYSGYYYTMDYWGQGTLVTVSDKTHTCPPCPAPE [symbol] GGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPA [symbol] IEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG.

The 1H3 hVH4 heavy chain variable domain is in italics. A hinge region is double underlined. The remaining sequence is derived from IgG1 Fc. L234F/L235E/P331S mutations in the IgG1 Fc domain bolded and dotted-underlined.

n. 1H3 hVH4-Fc (IgG1 Fc; FES)

One embodiment provides a fusion protein having 50, 60, 70, 80, 85, 90, 95, 96, 97, 98, 99, or 100% sequence identity to 1H3 hVH4 heavy chain variable domain Fc (IgG1 Fc; FES) having the following sequence:

(SEQ ID NO: 72)
QVQLQESGPGLVKPSETLSLTCTVSGFSLSNYGVHWVRQPPGKGLEWIGL
IWSEGSTTYASALKSRVTISKDTSKNQVSLKLSSVTAADTAVYYCARHPY
DDYSGYYYTMDYWGQGTLVTVSDKTHTCPPCPDKTHTCPPCPAPEFEGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPASIEKTISK
AKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQ
KSLSLSPG

The bolded and italicized sequence is the 1H3 hVH4 heavy chain variable domain. The double underlined sequence is a linker. The unmarked sequence is Fc Domain (IgG1 FES).

o. 1H3 hVH4-Fc (IgG4 Fc; G4P)

Another embodiment provides a fusion protein having 85%, 90%, 95%, or 100% sequence identity to 1H3 hVH4-Fc (IgG4 Fc; G4P) having the following sequence:

(SEQ ID NO: 73)
QVQLQESGPGLVKPSETLSLTCTVSGFSLSNYGVHWVRQPPGKGLEWIGL
IWSEGSTTYASALKSRVTISKDTSKNQVSLKLSSVTAADTAVYYCARHPY
DDYSGYYYTMDYWGQGTLVTVSESKYGPPCPPCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSLG
*.

The bolded and italicized sequence is the 1H3 hVH4 heavy variable domain. The double underlined sequence is the hG4P Fc (G4P sequence).

### 3. Siglec-15 Nucleic Acids and Cells

Vectors encoding Siglec-15 polypeptides, fragments and fusions thereof are also provided. Nucleic acids, such as those described above, can be inserted into vectors for expression in cells. Thus cells containing and expressing Siglec-15 polypeptides, fragments and fusions thereof are also provided. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, CT), maltose E binding protein and protein A. In some embodiments, a nucleic acid molecule encoding a Siglec-15 fusion polypeptide is present in a vector containing nucleic acids that encode one or more domains of an Ig heavy chain constant region, for example, an amino acid sequence corresponding to the hinge, C_{H}2 and C_{H}3 regions of a human immunoglobulin Cγ1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the Siglec-15 fusion polypeptides described herein.

The vectors described can be used to express Siglec-15 in cells. An exemplary vector includes, but is not limited to, an adenoviral vector. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject. In one embodiment, expression vectors containing nucleic acids encoding fusion proteins are transfected into cells that are administered to a subject in need thereof.

In vivo nucleic acid therapy can be accomplished by direct transfer of a functionally active DNA into mammalian somatic tissue or organ in vivo. For example, nucleic acids encoding polypeptides disclosed herein can be administered directly to lymphoid tissues or tumors. Alternatively, lymphoid tissue specific targeting can be achieved using lymphoid tissue-specific transcriptional regulatory elements (TREs) such as a B lymphocyte-, T lymphocyte-, or dendritic cell-specific TRE. Lymphoid tissue specific TREs are known in the art.

Nucleic acids may also be administered in vivo by viral means. Nucleic acid molecules encoding fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors.

Nucleic acids may also be delivered by other carriers, including liposomes, polymeric micro- and nanoparticles and polycations such as asialoglycoprotein/polylysine.

In addition to virus- and carrier-mediated gene transfer in vivo, physical means well-known in the art can be used for direct transfer of DNA, including administration of plasmid DNA and particle-bombardment mediated gene transfer.

### D. Pharmaceutical Compositions

Pharmaceutical compositions including the disclosed Siglec-15-binding molecules are provided. Pharmaceutical compositions containing a Siglec-15-binding molecule can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed Siglec-15-binding molecules, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally, for intravenous injection or infusion, dosage may be lower.

The dosage administered to a patient is typically 0.01 mg/kg to 100 mg/kg of the patient's body weight. The dosage administered to a patient can be, for example, between 0.01 mg/kg and 20 mg/kg, 0.01 mg/kg and 10 mg/kg, 0.01 mg/kg and 5 mg/kg, 0.01 and 2 mg/kg, 0.01 and 1 mg/kg, 0.01 mg/kg and 0.75 mg/kg, 0.01 mg/kg and 0.5 mg/kg, 0.01 mg/kg to 0.25 mg/kg, 0.01 to 0.15 mg/kg, 0.01 to 0.10 mg/kg, 0.01 to 0.05 mg/kg, or 0.01 to 0.025 mg/kg of the patient's body weight. Exemplary specific dosages include, but are not limited to 0.2 mg/kg, 0.3 mg/kg, 1 mg/kg, 3 mg/kg, 6 mg/kg or 10 mg/kg. A dose as low as 0.01 mg/kg is believed to be suitable to have appreciable pharmacodynamic effects. Dose levels of 0.10-1 mg/kg are predicted to be most appropriate. Higher doses (e.g., 1-30 mg/kg) would also be expected to be active.

Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In certain embodiments, the Siglec-15-binding molecule is administered locally, for example by injection directly into a site to be treated. Typically, the injection causes an increased localized concentration of the Siglec-15-binding molecule composition which is greater than that which can be achieved by systemic administration. The Siglec-15-binding molecule compositions can be combined with a matrix as described below to assist in creating an increased localized concentration of the polypeptide compositions by reducing the passive diffusion of the polypeptides out of the site to be treated.

### 1. Formulations for Parenteral Administration

In certain embodiments, compositions disclosed herein, are administered in an aqueous solution, by parenteral injection or infusion. The formulation may also be in the form of a suspension or emulsion. In general, pharmaceutical compositions are provided including effective amounts of a Siglec-15-binding molecule, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Controlled Delivery Polymeric Matrices

The Siglec-15-binding molecules disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of fusion polypeptides or nucleic acids encoding the fusion polypeptides. These may be natural or synthetic polymers. Synthetic polymers typically have a better characterization of degradation and release profiles. The polymer is selected based on the period over which release is desired. In some cases, linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### III. Method of Use

Methods of using the disclosed anti-Siglec 15 antibodies are provided here. Such antibodies may be used to reduce bone loss, increase bone formation, and improve new bone quality for persons with limited mobility or reduced mechanical loading due to limited mobility. Normal bone undergoes a continuous remodeling process triggered by weight-bearing activities that leads to bone degradation followed by new bone formation. Non-ambulatory or limited mobility patients are subject to significant bone loss due to reduced pressure and activity that is required for homeostatic bone maintenance. This often leads to greater risk for fracture in the spine, hip, and forearm. In older patients, hip fracture increases the mortality rate 3-4× within one year (Morri et al, Sci Rep, 9:18718 (2019)). Immobilization osteoporosis occurs as a result of a wide variety of causes, including but not limited to spinal cord injury, cerebral palsy, stroke, paralysis, comas, time spent at zero gravity (e.g. in outer space) and other medical conditions that lead to prolonged bed rest.

Bone loss is frequently diagnosed by bone mineral density (BMD) using dual energy X-ray absorptiometry (DXA). Osteoporosis is diagnosed as a DXA Z-score of lower than −2.5, which is indicative of lower bone mineral density than other healthy individuals in the same age group. While oral bisphosphonates may provide slight benefit to non-ambulatory patients, the effects are moderate at best and often do not lead to high quality bone generation. Hormonal therapies, while available, still do not address the ability to maintain bone with significantly reduced load-bearing activities, as discussed previously.

Spinal cord injury (SCI) causes extreme immobilization with immediate and irreversible unloading of the skeleton that leads to marked bone loss that can exceed 50% at the distal femur and proximal tibia (Qin et al, Curr Osteoporos Rep, 2010; Qin et al, Ann NY Acad Sci, 2010). In neurologically motor-complete SCI, bone loss proceeds at a rate of 1% per week for the first 6-12 months (Szollar et al, Am J Phys Med Rehabil, 1998; Garland et al, J Spinal Cord Med, 2004; Warden et al, Osteoporos Int, 2002), a rate that is substantially greater than that observed in post-menopausal women (3-5%/year), which is a non-immobilizing condition of bone loss with heightened awareness in the medical community (Recker et al, J Bone Miner Res 2000). While bone loss is most rapid in the first 6-12 months post injury, bone loss continues for at least 5 to 8 years, and possibly for decades. At present, there is no practical treatment to delay or prevent bone loss, or promote rebuilding of bone, in individuals with SCI. Bisphosphonates prevent osteoporosis in the elderly, but appear, at best, to transiently reduce SCI-related bone loss in individuals with SCI who are ambulatory; in those with motor-complete SCI, bisphosphonates have been shown to be of transient benefit at the hip but had no effect to preserve BMD at the knee, which is the site most prone to fracture (Bauman et al, J Rehabil Res Dev, 2005; Gilchrist et al, J Clin Endocrinol Metab, 2007; Bauman et al, J Bone Miner Metab, 2015). There are more than 275,000 persons with SCI patients in the United States, and approximately 12,000 new injuries annually. Of the more than 200,000 patients, only about 10 percent have been able to rehabilitate enough to return to work or school (Fehlings et al, J Neurotrauma, 28(8): p. 1329-33 (2011)). Therefore, development of a safe and effective therapy for bone boss after SCI is a high priority.

In addition, other medical conditions that also lead to non-ambulatory states have increased rates of osteoporosis, which is not adequately addressed by current therapeutics. A cross-sectional study on adults with cerebral palsy concluded that 25% of patients had low BMD at the lumbar spine and femoral neck, which had a higher correlation in the non-ambulatory versus ambulatory groups (Won and Jung et al, Front Neurol, 12 (2021)). Similarly, a study showed that non-ambulatory children and adults with spina bifida had a 9.8 times higher risk of fracture than those that were ambulatory (Trinh et al, Osteoporos Int, 28(1): p. 399-406 (2017)). Each of these debilitating conditions requires the development of novel therapeutics to address bone loss. An anti-Siglec-15 antibody could provide a safe, effective approach for decreasing bone resorption and increasing bone formation.

Outside of medical conditions, microgravity in space is another reason for reduced mechanical loading that leads to disuse osteoporosis. Cosmonauts at the International Space Station were studied to understand the effects of reduced mechanical load on bones due to space. After a few months in space, significant bone resorption and decreased BMD at the tibia were measured. Although cortical size and density recovered over the course of one year after landing, the trabecular bone did not recover, leading to increased bone fragility (Vico et al, J Bone Miner Res, 32(10): p. 2010-2021 (2017)). This study highlights the need for therapeutics to prevent initial bone loss and improve bone formation upon loss of mechanical loading (LeBlanc et al, J Musculoskelet Neuronal Interact, 7(1): p. 33-47 (2007)). The spinal cord injury-induced immobilization rodent models are frequently used to model bone loss due to microgravity. Studies suggest that osteoporosis due to microgravity are likely caused by increased bone loss rather than decrease in bone formation (Rolvien and Amling, Calif Tissue Int, 110(5): p. 592-604 (2022)). This wide variety of conditions described here all require therapeutics to address the bone loss due to non-ambulatory or reduced load-bearing states.

Sialic acid-binding immunoglobulin-like lectin (Siglec)-15 is expressed on the cell surface of mature osteoclasts (Kitagawa et al, J Biol Chem, 2012). Numerous studies have identified the critical role for Siglec-15 on osteoclastogenesis using a knock-out mouse model (Hiruma et al, Bone, 2013; Kameda et al, Bone, 2015). Mice lacking Siglec-15 are largely unable to fuse pre-osteoclast cells to form mature osteoclasts, which leads to decreased bone resorption, and thus increased bone formation. The phenotypes of the Siglec-15 null mouse include abnormal osteoclast differentiation and osteopetrosis, which is defined by abnormal bone growth and increased bone mineral density (Zhen et al, Bone Res, 2021). Since Siglec-15 is expressed on osteoclasts to promote fusion, inhibiting this fusion could lead to increase in mononuclear osteoclasts that are reported to have high potency to support bone formation. Thus, inhibiting Siglec-15 to prevent osteoclast fusion could have both anti-resorptive as well as anabolic effects on bone, without requiring load-bearing weight to promote healthy bone homeostasis.

### A. Therapeutic and Prophylactic Uses

Therapeutic and/or prophylactic use of antibodies or their antigen-binding fragments that specifically bind human Siglec-15 and that are capable of reducing the binding of Siglec-15 to one or more of its ligands or counter-receptors are provided.

An antibody that reduces the binding of Siglec-15 to its ligand(s) or a counter-receptor can lead to reduced signaling within the cell that Siglec-15 is expressed on. Additionally, blocking the binding of Siglec-15 may lead to reduced signal transduction through the counter-receptor. The disclosed molecules may block Siglec-15 binding to various degrees and at different epitopes of Siglec-15, thus having slightly different functional effects. As mentioned above, blocking Siglec-15 in pre-osteoclasts may contribute to inhibit bone loss and simultaneously catalyze new bone formation. Blocking Siglec-15 inhibits the fusion of mature osteoclasts and leads to accumulation of pre-osteoclasts that secrete the growth factor PDGF-BB (Platelet-derived growth factor-BB). This growth factor is critical for recruitment of osteoblasts and angiogenesis, which are two key components of new bone formation (Xie et al, Nat Med, 20, 1270-1278 (2014); Zhen et al, Bone Research, 2021).

### B. Subjects to be Treated

### 1. Treatment of Osteoporosis Due to a Non-Ambulatory State or Reduction of Mechanical Load Bearing.

Anti-Siglec-15 antibodies disclosed here may be used to treat osteoporosis or to prevent bone loss that results from a non-ambulatory state. As described above, normal bone homeostasis relies on the gravity-dependent load-bearing state to promote bone resorption and new bone formation. Situations where the load-bearing state is compromised include, but not limited to, spinal cord injury, hip fracture, cerebral palsy, stroke, paralysis, comas, time spent at zero gravity (e.g., in outer space) and other medical conditions that lead to prolonged bed rest. This would also include any disease or syndromes by which a person is dependent on a wheelchair for mobility purposes, including but not limited to Guillain-Barre syndrome, Ehlers-Danlos syndrome, Multiple Sclerosis, Muscular Dystrophy, Amyotrophic Lateral Sclerosis, Spina bifida, polio, Parkinson's disease, or activity-limiting rheumatological diseases. This may include people that are non-ambulatory and have a bone mineral density T-score or Z-score less than or equal to −2.0. A bone mineral T-score is the deviation of bone strength from the average 30-year-old. A Z-score reflects the standard deviation of bone strength from a matched age and gender population.

In each of these conditions, there is reduced load bearing, which ultimately leads to increased bone loss. With use of the method described here, treatment with an anti-Siglec-15 antibody would block osteoclast maturation, thus accumulating pre-osteoclasts. As overall bone resorption would decrease due to the lack of mature osteoclasts, pre-osteoclasts will continue to regulate osteoblasts, thus promoting new bone formation. Therapeutic blocking of Siglec-15 would balance bone homeostasis, even in the absence of mechanical loading of the bone.

### C. Combination Therapies

The disclosed Siglec-15-binding and Siglec-15 ligand-binding molecules can be administered to a subject in need thereof alone or in combination with one or more additional therapeutic agents. In some embodiments, the Siglec-15-binding or Siglec-15 ligand-binding molecule and the additional therapeutic agent are administered separately, but simultaneously. The Siglec-15-binding or Siglec-15 ligand-binding molecule and the additional therapeutic agent can also be administered as part of the same composition. In other embodiments, the Siglec-15-binding or Siglec-15 ligand-binding molecule and the second therapeutic agent are administered separately and at different times, but as part of the same treatment regime.

The subject can be administered a first therapeutic agent 1, 2, 3, 4, 5, 6, or more days, or 1, 2, 3, 4, 5, 6, 7, or more months before administration of a second therapeutic agent. In some embodiments, the subject can be administered one or more doses of the first agent every 1, 2, 3, 4, 5, 6 7, 14, 21, 28, 35, or 48 days prior to a first administration of second agent. The Siglec-15-binding or Siglec-15 ligand-binding molecule can be the first or the second therapeutic agent. In some embodiments, one or more Siglec-15 binding molecules and one or more Siglec-15 ligand-binding molecules are administered in combination.

The Siglec-15-binding and/or Siglec-15 ligand-binding molecule and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

Exemplary molecules include, but are not limited to bisphosphonates, cytokines, other immunotherapeutics, enzymes, antibiotics, growth factors, growth inhibitors, hormones (including testosterone and parathyroid hormone), hormone antagonists, antibodies and bioactive fragments thereof (including humanized, single chain, and chimeric antibodies), antigen and vaccine formulations (including adjuvants), peptide drugs, anti-inflammatories, molecules that target additional bone metabolic pathways and modulate osteoclastogenesis or osteoblastogenesis. The additional therapeutic agents are selected based on the condition, disorder or disease to be treated. For example, the Siglec-15-binding molecule can be co-administered with one or more additional agents that function to enhance bone formation or inhibit bone loss.

Male hormone testosterone is an important determinant of bone mass and metabolism through its direct effects on osteoblasts and osteocytes (Notelovitz, Fertil Steril, 2002; Francis, Clin Endocrinol, 1999; Anderson et al, Calif Tissue Int, 1998; Pederson et al, Proc Natl Acad Sci USA, 1999; Zaidi, Nat Med, 2007; Saita et al, Horm Metab Res, 2009). The levels of circulating testosterone are commonly reduced after acute SCI and in those with chronic injury (Tsitouras et al, Horm Metab Res, 1995; Schopp et al, Am J Phys Med Rehabil, 2006; Bauman et al, J Spinal Cord Med, 2014). More than 80% of SCI patients are men (Qin et al, Ann NY Acad Sci, 2010). Absolute or relative hypogonadism would be expected to accelerate bone loss after SCI (Qin et al, Ann NY Acad Sci, 2010; Tsitouras et al, Horm Metab Res, 1995; Kostovski et al, Spinal Cord, 2008). Recently, we found that nandrolone, an anabolic steroid, reduced bone loss after SCI (Sun et al, J Spinal Cord Med, 2013) or after nerve transaction (Cardozo et al, Ann NY Acad Sci, 2010). Interestingly, nandrolone increased the expression of OPG and Runx2, and activated the Wnt signaling pathway, in bone marrow-derived osteoblasts from SCI rats (Sun et al, J Spinal Cord Med, 2013). These findings suggest that one effect of nandrolone on unloaded bone is to stimulate Wnt signaling in osteoblasts and promote bone formation. Because Wnt signaling is anabolic to bone, these findings raise the possibility that this action of nandrolone may underlie some or all of its protective effects on bone after SCI. If this is the case, it is notable that androgen acts through a distinct and complimentary mechanism from that of anti-Siglec-15 antibody. Thus, synergistic effects to bone may occur when anti-Siglec-15 antibodies and an androgen are combined as a dual therapeutic approach.

As described above, Denosumab (anti-RANKL antibody) and bisphosphonates have been used clinically to inhibit osteoclast maturation and activity. As these therapeutics act through a distinct mechanism than that of anti-Siglec-15, they may be used in combination with anti-Siglec-15 to generate a synergistic effect on bone formation. While Denosumab and bisphosphonates have an effect on early osteoclast development, anti-Siglec-15 targets the pre-osteoclasts that allows for better regulation between osteoclasts and osteoblasts. Thus, a combination of the two could help further reduce bone resorption while also providing increased bone formation. In addition, the anti-Sclerostin antibodies that have a similar anti-resorptive and anabolic effect on bone may be used in combination with an anti-Siglec-15 antibody. Since the use of anti-Sclerostin antibodies has been associated with cardiac toxicities, a potential treatment regimen may begin with the anti-Sclerostin, followed by an anti-Siglec-15 antibody to target bone loss through a mechanistically distinct pathway. The sclerostin antibodies specifically target the Wnt signaling pathway in the osteoblasts, while Siglec-15 antibodies target osteoclast maturation. Given the distinct signaling mechanisms, a combination of the two therapeutics could be beneficial.

### IV. Diagnostic Methods

The Siglec-15-binding molecules, particularly antibodies and their antigen-binding fragments, can be used for diagnostic purposes, such as to detect, diagnose, or monitor diseases, disorders or infections associated with Siglec-15 expression, or to determine or assist in the determination or identification of suitable patient populations or profiles. Any of the methods can be coupled with a method of treating the subject, for example, by administering the subject an effective amount of one or more therapeutic Siglec-15-binding molecules.

The detection or diagnosis of a disease, disorder or infection, including, but not limited to, cancer can include: (a) assaying the expression of Siglec-15 or derivatives thereof in cells, serum, plasma, blood or in a tissue sample (e.g., a tumor sample) of a subject using one or more antibodies (or fragments thereof) that immunospecifically bind to such antigens; and (b) comparing the level of the antigen with a control level, e.g., levels in normal tissue samples, whereby an increase in the assayed level of antigen compared to the control level of the antigen is indicative of the disease, disorder or infection. Such antibodies and fragments can be employed in immunoassays, such as the enzyme linked immunosorbent assay (ELISA), the radioimmunoassay (RIA) and fluorescence-activated cell sorting (FACS).

In some embodiments, the antibodies or fragments are used for IHC analysis in cells of an in vitro or in situ tissue sample or in vivo. Thus, the antibodies and fragments can be used in the detection and diagnosis of a disease, disorder, or infection in a human. In one embodiment, such diagnosis includes: a) administering to a subject (for example, parenterally, subcutaneously, or intraperitoneally) an effective amount of such labeled antibody or antigen-binding fragment; b) waiting for a time interval following the administration for permitting the labeled molecule to preferentially concentrate at sites in the subject where Siglec-15 is expressed (and for unbound labeled molecule to be cleared to background level); c) determining background level; and d) detecting the labeled antibody in the subject, such that localized detection of labeled antibody above or below the background level indicates that the subject has the disease, disorder, or infection and/or shows the location and relative expression level of Siglec-15+ tissue. In accordance with this embodiment, the antibody can be labeled with an imaging moiety which is detectable in vivo using an imaging system known to one of skill in the art. Background level can be determined by various methods including, comparing the amount of labeled molecule detected to a standard value previously determined for a particular system.

Other methods include, for example, monitoring the progression of a disease, disorder or infection, by (a) assaying the expression of Siglec-15 in cells or in a tissue sample of a subject obtained at a first time point and later time point using a Siglec-15-binding molecule and (b) comparing the level of expression of Siglec-15 in the cells or in the tissue sample of the subject at the first and later times points, wherein an increase in the assayed level of Siglec-15 at the later time point compared to the first time point is indicative of the progression of disease, disorder or infection.

A method for monitoring a response to a treatment, can include, (a) assaying the expression of Siglec-15 in cells or in a tissue sample of a subject prior and after the treatment using a Siglec-15-binding molecule; and (b) comparing the level of Siglec-15 over time, whereby a decrease in the assayed level of Siglec-15 after treatment compared to the level of Siglec-15 prior to treatment is indicative of a favorable response to the treatment.

It will be understood in the art that the size of the subject and the imaging system used will determine the quantity of imaging moiety needed to produce diagnostic images.

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In one embodiment, monitoring of a disease, disorder or infection is carried out by repeating the method for diagnosing the disease, disorder or infection, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that can be used in the diagnostic methods include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### V. Kits

The disclosed Siglec-15-binding or Siglec-15 ligand-binding molecules can be packaged in a hermetically sealed container, such as an ampoule or sachette, indicating the quantity. The molecules can be supplied as a dry sterilized lyophilized powder or water free concentrate in a hermetically sealed container and can be reconstituted, e.g., with water or saline to the appropriate concentration for administration to a subject. For example, the molecules can be supplied as a dry sterile lyophilized powder in a hermetically sealed container at a unit dosage of at least 5 mg, or at least 10 mg, at least 15 mg, at least 25 mg, at least 35 mg, at least 45 mg, at least 50 mg, or at least 75 mg. The lyophilized molecules can be stored at between 2 and 8° C. in their original container and are typically administered within 12 hours, or within 6 hours, or within 5 hours, or within 3 hours, or within 1 hour after being reconstituted.

In an alternative embodiment, molecules supplied in liquid form in a hermetically sealed container indicating the quantity and concentration. In some embodiments, the liquid form of the molecules supplied in a hermetically sealed container including at least 1 mg/ml, or at least 2.5 mg/ml, at least 5 mg/ml, at least 8 mg/ml, at least 10 mg/ml, at least 15 mg/ml, at least 25 mg/ml, at least 50 mg/ml, at least 100 mg/ml, at least 150 mg/ml, at least 200 mg/ml of the molecules.

Pharmaceutical packs and kits including one or more containers filled with Siglec-15-binding or Siglec-15 ligand-binding molecules are also provided. Additionally, one or more other prophylactic or therapeutic agents useful for the treatment of a disease can also be included in the pharmaceutical pack or kit. The pharmaceutical pack or kit can also include one or more containers filled with one or more of the ingredients of the disclosed pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

Kits designed for the above-described methods are also provided. Embodiments typically include one or more Siglec-15-binding or Siglec-15 ligand-binding molecules. In particular embodiments, a kit also includes one or more other prophylactic or therapeutic agents useful for the treatment of cancer, in one or more containers.

### EXAMPLES

### Example 1: SIGLEC-15 Antibodies and Heavy and Light Chains Sequences Thereof

### Materials and Methods

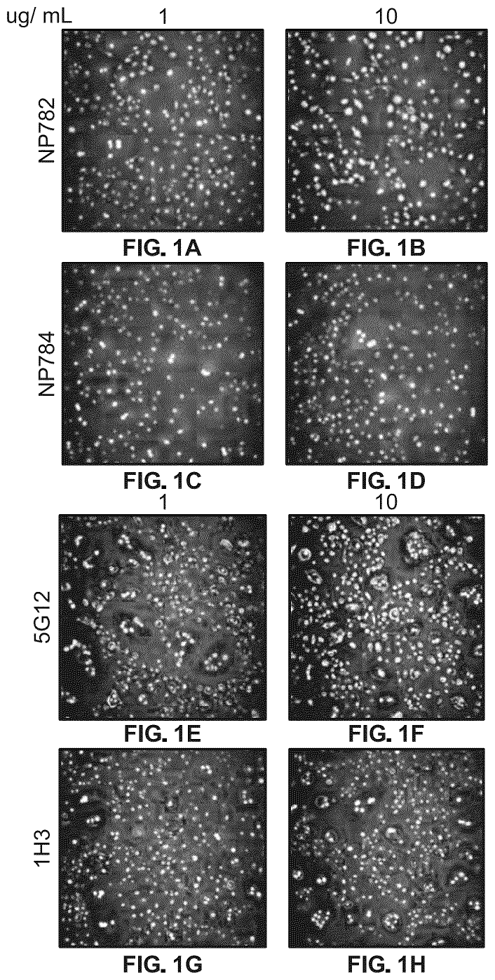

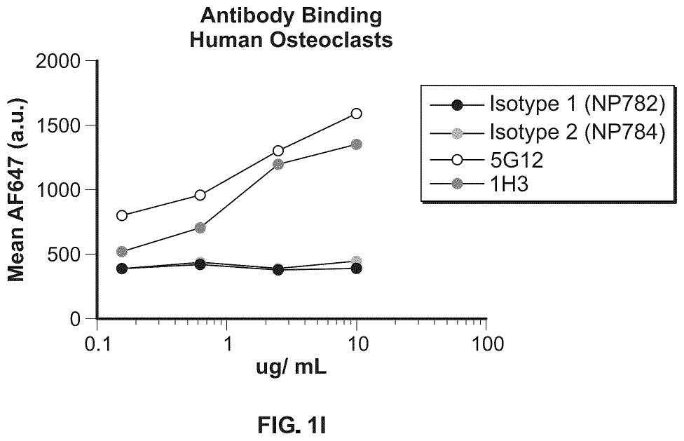

Mouse anti-human Siglec-15 monoclonal antibodies: Siglec-15 knock out mice (n=2) were immunized with hS15.mIg (human Siglec 15 extracellular domain [ECD] fused with mouse IgG2a) emulsified with CFA (complete Freund adjuvant). Mice also received injection of GM-CSF and anti-CD40. Mice were challenged with the same immunogen 2 weeks later. Antisera titer was assessed by testing serum collected from tail bleeding in hS15.hIg (human Siglec 15 ECD fused with human IgG1) coated ELISA plate at various dilution, 1:1000 up to 1:100,000,000. FIG. 1 shows that anti-hS15 antibodies were detected at >1:100,000 dilution. Mice received a 3^{rd} dose of antigen two weeks later. Three days after the final boost, mouse splenocytes were harvested and resuspended in RPMI supplemented with 10% FBS and glutamine, and later fused to form hybridomas.

Electrofusion of Siglec-15 knockout (S15 KO) splenocytes: Fused cells were plated in methylcellulose gel/media; left over fused cells were cryopreserved and can be thawed for another batch of cloning. Single clones were picked and placed in 10×96 well plates (960 clones). Supernatant were collected 2 weeks later.

RACE: RACE (Rapid Amplification of cDNA Ends) identification of the heavy and light chains was performed according to the following protocol: (1) mRNA denaturing, (2) cDNA synthesis, (3) 5′RACE Reaction, (4) analyzed PCR results (on an agarose gel to visualize the amplified DNA fragment—the correct antibody variable region DNA fragments should have a size between 500-700 base pairs, (5) TOPO cloned PCR positive bands; (6) PCR-amplified TOPO clones, followed by gel electrophoresis and recovery from agarose gel, (7) sequenced 218 clones in total, (8) performed CDR analysis using sequencing data (CDR regions were defined using VBASE2).

### Results

Antibodies were cloned using RACE methods. After sequencing 218 cloned DNA fragments, antibody sequence analysis identified one heavy chain and one light chain for antibody samples referred to herein as 1H3 and 5G12. The antibody nucleotide sequences, heavy and light chain sequences and CDRs are provided as SEQ ID NOs: 3-48 above. Humanized antibodies are provided as SEQ ID NO: 8, 9, 10, 16, 17, 19, 21, 28, 29, 31, 33, 35, 42, 45 and 46. Fusion proteins are provided as SEQ ID NO: 49, 50, 51, 52, 53, and 55.

### Example 2: Anti-huS15 Antibodies Bind to Cells Expressing Human S15 or Mouse S15

### Materials and Methods

Human osteoclasts were differentiated from monocytes isolated from fresh human PBMCs. StemCell monocyte isolation kit was used to negatively select for monocytes, which were cultured in complete RPMI (RPMI, 10% FBS, 2% glutamine, 2% HEPES, 2% sodium pyruvate, 2% Penicillin and Streptomycin, and 2% Normocin) with 25 ng/mL human recombinant MCSF. After four days of culture, human recombinant RANKL was added at 66 ng/mL. After 4 to 7 days, cells are washed in FACS buffer (PBS with 2% FBS), blocked with Human TruStainFx at 1:100, and stained with anti-Siglec-15 antibodies conjugated with AlexaFluor 647. Cells were incubated with antibodies for 1 hour on ice, washed twice in FACS buffer, and imaged on the Opera Phenix to determine binding of antibodies to osteoclasts.

Mouse osteoclasts were generated from differentiating RAW 264.7 macrophages with MCSF and RANKL for 3 days.

### Results

Anti-human Siglec15 antibodies including 1H3 and 5G12 were tested for binding to in vitro differentiated human osteoclasts. The results are illustrated in FIGS. 1A-1I. Both antibodies bind to osteoclasts in a dose titration manner in comparison to the control isotype antibodies (NP782 and NP784).

### Example 3: Anti-Siglec-15 Antibody Significantly Prevented Sublesional Bone Loss at Distal Femur and Proximal Tibia in a Rat Model of Severe Immobilization Secondary to Acute SCI

### Materials and Methods

Male Wistar rats (275 g) underwent a complete spinal cord transection (T3-T4). A laminectomy was performed on controls for a “sham” surgery. Immediately after spinal cord transection, SCI rats were treated with vehicle or anti-muSiglec-15 antibody at 20 mg/kg, once every two weeks for eight weeks before sacrifice. Bone loss and formation was measured by bone mineral density (BMD), bone mineral content (BMC), and histomorphometry. The effects of the anti-Siglec-15 antibody was also measured by numbers of osteoclasts and osteoblasts isolated from the bone marrow and gene expression changes in these cells to determine alterations in differentiation and activity. The groups studied included:
Groups 1: Sham SCI, vehicle (0.9% saline) (15 animals)
Groups 2: SCI, vehicle (0.9% saline) (15 animals)
Groups 3: SCI, anti-muSiglec-15 (SEQ ID NO: 3 and 11) at 20 mg/kg/biweekly (15 animals)

Spinal cord transection: A well-established model of SCI in rodents was used for this study. These animals have been engineered to display extensive sublesional bone loss in the knee joint, analogous to the clinical setting. Spontaneous emptying of the bladder function developed within a few weeks after SCI. The survival rate was greater than 90%. This preclinical model of bone loss has been successfully used to evaluate the efficacy of several interventions, including androgen, sclerostin antibody, and electrical stimulation (Sun et al, J Spinal Cord Med, 36(6):616-22 (2013); Qin et al, J Bone Miner Res, 2015; Qin et al, J Biol Chem, 288(19):13511-21 (2013)). Briefly, animals were anesthetized by inhalation of isofluorane, hair was removed with a clipper, and skin over the back was cleaned with betadine and isopropyl alcohol. After making a midline incision the spinal cord at the site of transection, viewing the area by laminectomy, the spinal cord was transected with microscissors. The space between the transected ends of the spinal cord was filled with surgical sponge and the wound was closed in two layers with suture. Urine was expressed three times daily until automaticity developed, then as needed. Baytril was administered for the first three to five days postoperatively then as indicated for cloudy or bloody urine or for overt wound infection. Sham-transected animals received an identical surgery, including a laminectomy, except that the spinal cord was not cut. Rats were injected with saline or 20 mg/kg anti-muSiglec-15 subcutaneously (s.c) once every two weeks for eight weeks.

Double labeling: Newly formed bone was labeled with fluorochromes by subcutaneous injection of calcein (10 mg/kg) and xylenol orange (90 mg/kg) on days −6 and −2 before euthanasia.

Euthanasia and Sample Collection: Animals were anesthetized by inhalation of isofluorane. Blood was collected by cardiac puncture and centrifuged to separate serum from whole blood, and stored at −20° C. The lumbar spine was dissected out and hindlimb was removed using sterile technique and careful dissection to free the head of the femur from the pelvis. Hindlimbs and lumbar spine were immediately imaged for DXA scanning then processed for additional analysis by one of the following procedures:
a) A subset of bone samples (left femur and tibia, N=6-7/group) were fixed in 10% formalin for 48 h and stored in 70% ethanol for micro-CT, SEM, histomorphometry and immunohistochemistry.
b) A subset of bone samples (right femur, N=6-7/group) were wrapped with gauze soaked with PBS and stored at −20° C. for bone mechanical testing (bone strength).
c) A subset of bones (left and right femur and tibia, N=5 per group) were placed into sterile tubes (50 ml) containing ice-cold Minimum Essential Alpha Medium (MEM, 30 ml), then kept at 4° C. until collection of marrow cells. For primary culture of marrow progenitor cells, bone samples were flushed out with MEM medium to obtain bone marrow cells and processed for ex vivo osteoblastogenesis and osteoclastogenesis assays.

### Bone Biology Analyses:

a) Determinations of areal bone mineral content (BMC) and BMD of the distal femur and proximal tibia was performed by DXA scanning.
b) Bone architecture was assessed by microCT scanning.
c) Blood levels of markers of bone resorption (e.g., CTX) and formation (e.g., Osteocalcin, OCN) by ELISA.
d) Histomorphometry was performed to determine rates of bone formation (double-labeling), and surface area for osteoblasts and osteoclasts (AP and TRAP staining).
e) Osteoblastogenesis and osteoclastogenesis assays: Osteoblasts (OB) and osteoclasts (OC) differentiation assays (CFU-F and CFU-OB for OB, TRAP staining for OC) were used to determine the numbers of osteoblasts and osteoclasts.
f) Real-time PCR was used to determine the expression of differentiation markers (e.g., TRAP, calcitonin receptor, and miR-183) for osteoclasts.
g) Measurement of bone strength by mechanical testing (4 points bending test). Femurs were loaded into the loading block anterior surface down and the distal end on the right of the block. The loading block position was adjusted at 0.03 mm/sec at a sampling rate for load of 25 samples per second until a load of 1500 N was achieved or a fracture occurs (decrease in load to 50% of previous maximum). MatLab was used to analyze the data to calculate failure load and stiffness (Chandrasekhar et al, Calif Tissue Int, 89(5): p. 347-57 (2011)).
h) These approaches to determine the changes of bone biology have been described in previous publications in greater detail (Qin et al, J Bone Miner Res, (2015); Qin et al, Osteoporos Int, 2016; Sun et al, J Spinal Cord Med, (2013); Cardozo et al, Ann NY Acad Sci 1192: p. 303-6 (2010), Qin et al, J Biol Chem, (2013); Bramlett et al, Osteoporos Int, 25(9):2209-19 (2014)).

### Results

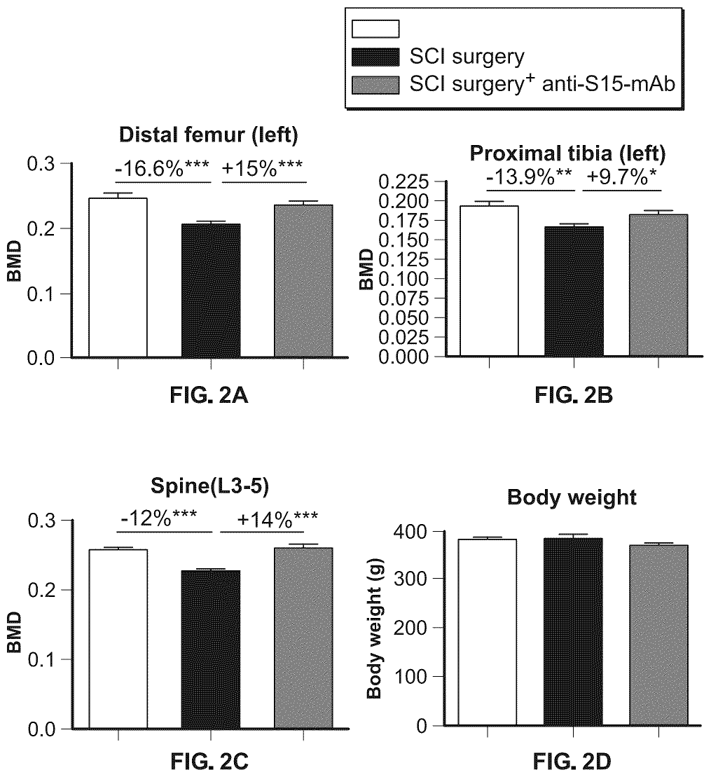

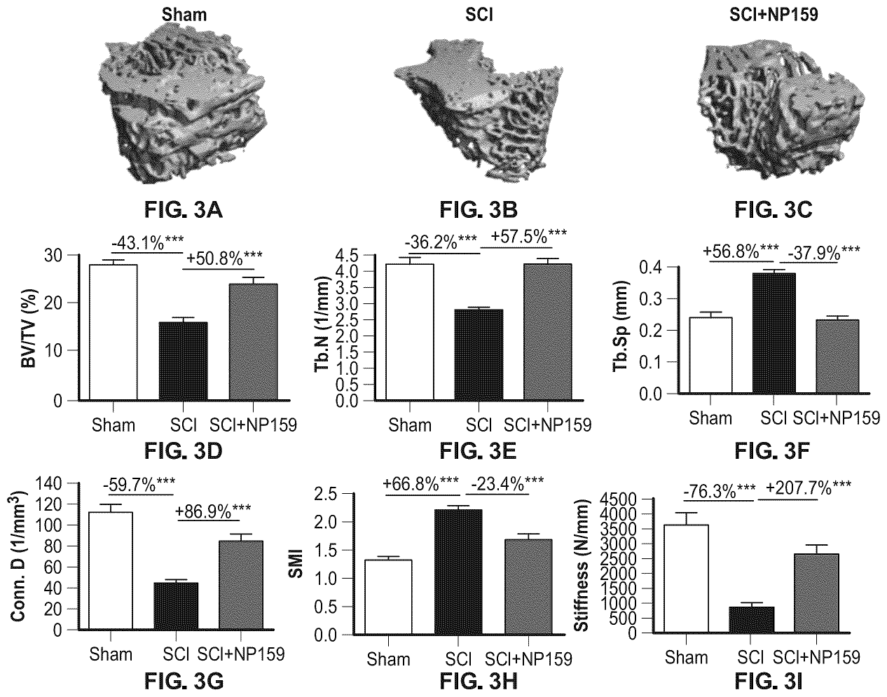

Effects of anti-Siglec-15 antibody on Bone Mass and Bone Micro-architecture: Eight weeks after SCI, bone mineral density (BMD) of the distal femur and proximal tibia were diminished by 16.6% and 13.9%, respectively. Of note, anti-Siglec-15 antibody treatment completely prevented the loss of BMD at these two skeletal sites after acute SCI (FIG. 2A-2B). High-resolution microCT analysis (FIGS. 3A-3I) revealed that after SCI, trabecular bone volume at the distal femur was reduced by 43.1% due largely to decreased trabecular number with an increase in trabecular space (FIG. 3D). Trabecular connectivity was greatly reduced and associated with transformation from plate-like to rod-like structures. Administration of the therapeutic almost completely prevented the declines in trabecular bone volume and connectivity, primarily by increasing trabecular number and preserving trabecular space. While SCI resulted in significantly decreased bone stiffness (by 76.3%) by finite element analysis, anti-Siglec-15-treated SCI rats dramatically increased bone stiffness by 207.7% compared to SCI-vehicle animals (FIG. 3I).

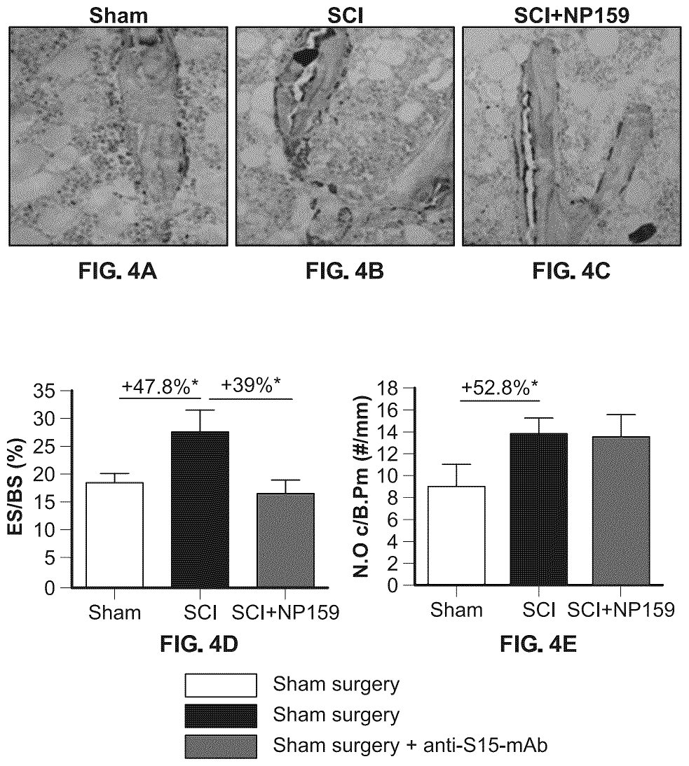

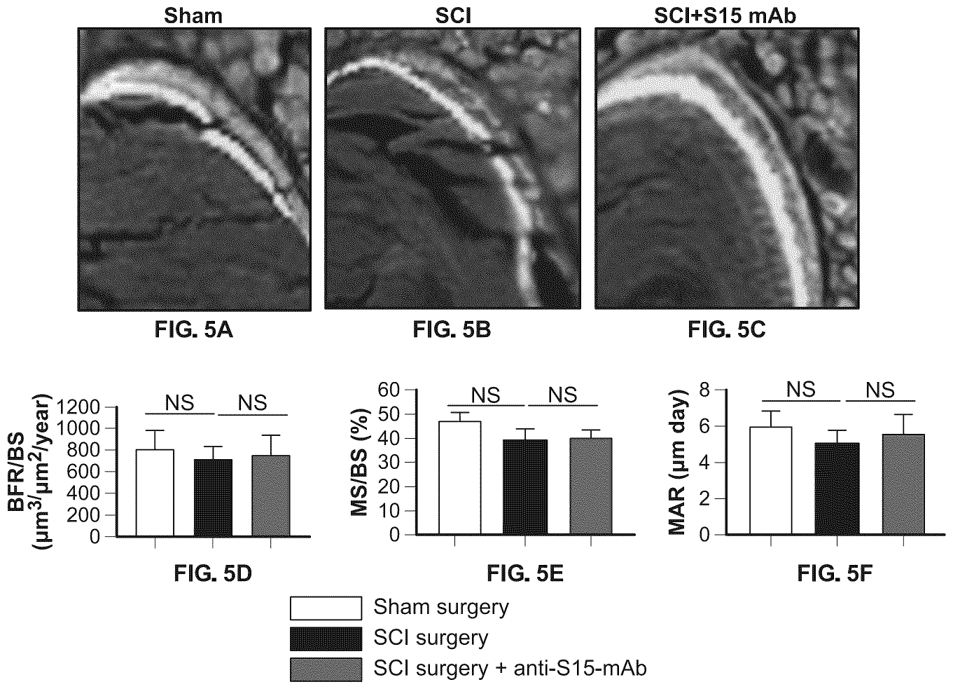

Effects of anti-Siglec-15 antibody on Bone Resorption and Bone Formation: Static histomorphometric analysis was conducted to evaluate bone resorption. Sections of trabecular bone from the femur were immuno-stained for TRAP (tartrate-resistant acid phosphatase, a marker for osteoclasts) (FIGS. 4A-4C). SCI resulted in increased eroded surface/bone surface (ES/BS (FIG. 4D)) and osteoclast number (N.Oc/B.Pm (FIG. 4E)). anti-Siglec-15 treatment in SCI rats greatly reduced ES/BS (39%) when compared with controls, suggesting inhibition of bone resorption. Because TRAP staining can detect both mononuclear (immature) and multinuclear osteoclasts (MUC, mature), no obvious changes of N.Oc/B.Pm in SCI+ treatment were detected, suggesting that anti-Siglec-15 mainly reduces the number of functional MUC for resorbing bone, but does not alter the total number of mononuclear pre-osteoclasts and osteoclasts (FIGS. 4A-4E), an observation that is consistent with previous studies (Sato et al, Bone, 135:115331 (2020); Zhen et al, Bone Res, 9(1):47 (2021)). Dynamic histomorphometric analysis (FIGS. 5A-5F) revealed no significant changes in MS/BS (FIG. 5E), MAR (FIG. 5F) and BFR/BS (FIG. 5D) in treatment group when compared to SCI group. These results indicate that treatment with an anti-Siglec-15 antibody maintains bone formation while inhibiting bone resorption, in contrast to other anti-resorptive drugs (e.g., bisphosphonates and Denosumab) which suppress bone formation (Tsukazaki et al, Bone, 152:116095 (2021)).

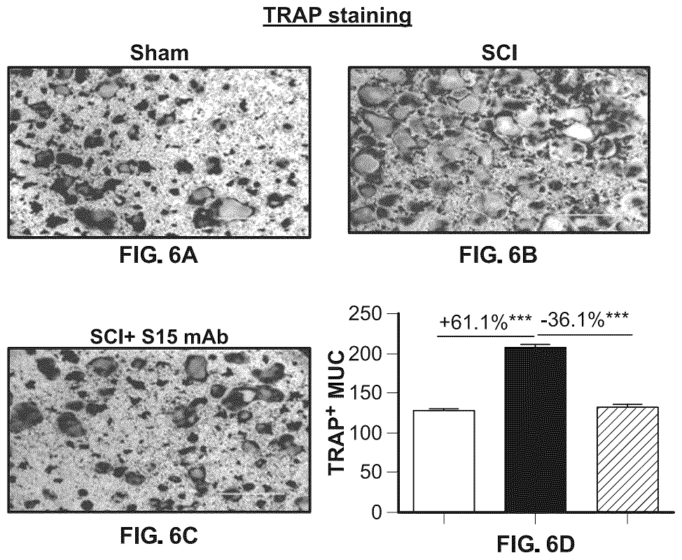

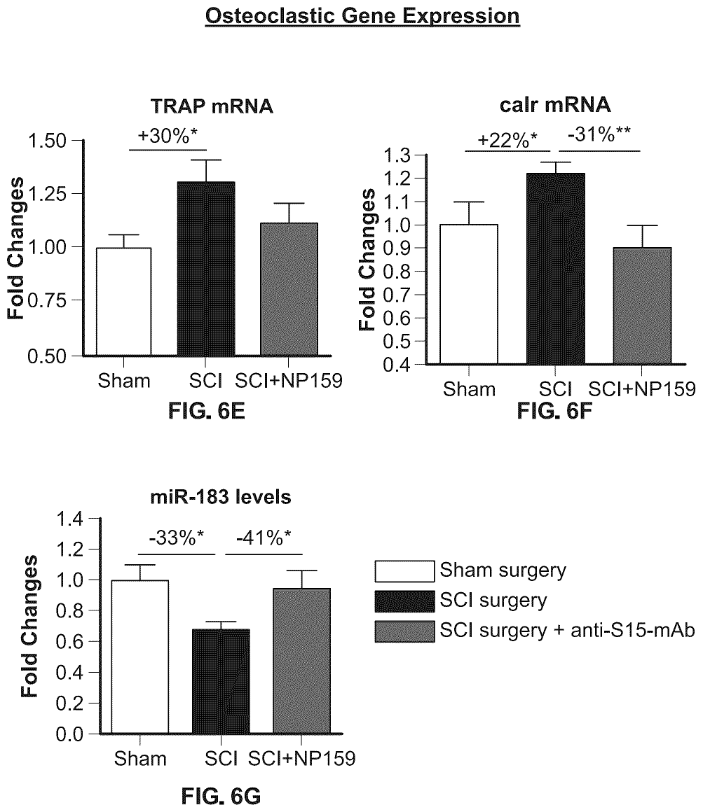

Effects of anti-Siglec-15 antibody on Osteoclastogenesis: Bone marrow cells can be separated into HPC and MSC populations which can then be differentiated into multinucleated TRAP⁺ osteoclasts capable of resorbing bone and alkaline phosphatase (ALP) positive osteoblasts that form and mineralize osteoid. Following immobilization, the potential of MSCs to undergo osteoblastogenic differentiation is reduced (Kondo et al, J Biol Chem, 280(34):30192-200 (2005); Basso et al, Bone, 37(3):370-8 (2005)). At 21 or 56 days after SCI, marrow HPCs demonstrate an increased ability for differentiation into osteoclasts (Qin et al, J Bone Miner Res, (2015); Qin et al, Osteoporos Int, (2016); Jiang et al, Osteoporos Int, 18(3):339-49 (2007)). We thus performed osteoclastogenesis and osteoblastogenesis assays to examine the effects of anti-Siglec-15 antibody on the ability of marrow cells to differentiate in cell cultures into osteoclasts or osteoblasts (FIGS. 6A-6G). Consistent with our previous findings (Qin et al, J Bone Miner Res, 2015; Qin et al, J Biol Chem, 2013; Bramlett et al, Osteoporos Int, 2014), after SCI, both the number of TRAP⁺ MUC (+61.1%) and the expression of osteoclast marker genes TRAP and calcitonin receptor (Calr) were significantly increased (FIGS. 6A-6G). Of note, treatment with the anti-Siglec-15 antibody markedly decreased TRAP⁺ MUC (−36.1% (FIG. 6D)) and TRAP & Calr mRNAs (FIG. 6E-6F), indicating the reduction of osteoclast maturation and osteoclastogenesis.

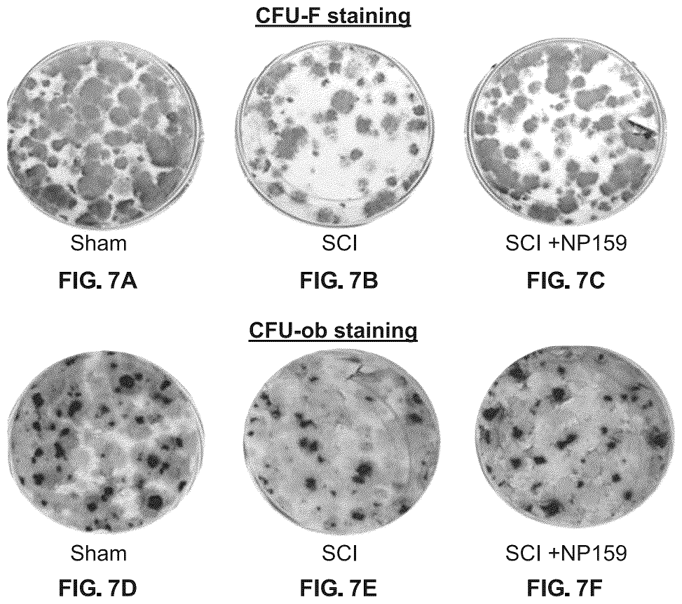

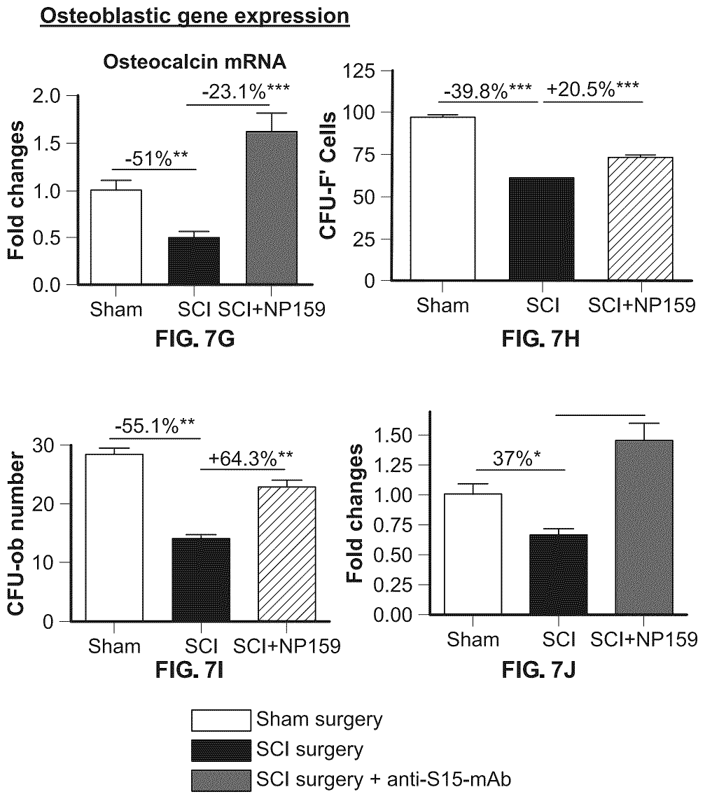

Effects of 1H3 on Osteoblastogenesis: Consistent with our previous findings (Qin et al, J Bone Miner Res, (2015); Zhao et al, Calif Tiss Int, 2018; Sun et al, J Spinal Cord Med, 2013), the number of osteoblast-forming cells (examined by CFU-F staining (FIGS. 7A-7C, 7G)), the number of colonies producing mineralized bone matrix (examined by von Kossa staining, CFU-ob (FIGS. 7D-7F, 7H), and the levels for transcripts encoding the osteoblast differentiation markers osteocalcin (OCN (FIG. 7I)) and BSP (FIG. 7J) were all decreased in rats following SCI. Notably, in anti-Siglec-15 antibody-treated SCI rats, there was a significant increase in the numbers of CFU-F⁺ staining (20.5%) or mineralized nodules (CFU-ob) cells (64.3%). In ex vivo cultures of osteoblasts derived from MSCs, mRNA levels of OCN and BSP were significantly increased in the SCI+1H3 group compared to SCI group (FIGS. 7I-7J). Collectively, our data indicates that anti-Siglec-15 antibody induced osteoblastogenesis while reducing osteoclast maturation and osteoclastogenesis (FIGS. 6A-6G).

In summary, treatment with the anti-Siglec-15 antibody fully prevented sublesional loss of BMD and metaphysis trabecular bone volume and preserved bone strength in a rat model of acute SCI. Because of its unique ability to reduce osteoclastogenesis and bone resorption while promoting osteoblastogenesis to maintain bone formation, the Siglec-15 Ab holds greater promise than prior anti-resorptive agents alone to mitigate the striking bone loss associated with SCI or other forms of severe immobilization.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### REFERENCES

Anderson, F. H., et al., Sex hormones and osteoporosis in men. Calcif Tissue Int, 1998. 62(3): p. 185-8.
Asselin P, Spungen A M, Muir J W, Rubin C T, Bauman W A. Transmission of low-intensity vibration through the axial skeleton of persons with spinal cord injury as a potential intervention for preservation of bone quantity and quality. J Spinal Cord Med. 2011; 34(1):52-9. Epub 2011/05/03. PubMed PMID: 21528627; PubMed Central PMCID: PMC3066482.
Basso N, Jia Y, Bellows C G, Heersche J N. The effect of reloading on bone volume, osteoblast number, and osteoprogenitor characteristics: studies in hind limb unloaded rats. Bone. 2005; 37(3):370-8. Epub 2005/07/12. doi: 10.1016/j.bone.2005.04.033. PubMed PMID: 16005699.
Battaglino R, Lazzari A, Garshick E, Morse L. Spinal Cord Injury-Induced Osteoporosis: Pathogenesis and Emerging Therapies. Current Osteoporosis Reports. 2012; 10(4):278-85. doi: 10.1007/s11914-012-0117-0. PubMed PMID: 22983921; PubMed Central PMCID: PMC3508135.
Bauman W A, Spungen A M, Wang J, Pierson R N, Jr., Schwartz E. Continuous loss of bone during chronic immobilization: a monozygotic twin study. Osteoporos Int. 1999; 10(2):123-7.
Bauman W A, Wecht J M, Kirshblum S, Spungen A M, Morrison N, Cirnigliaro C, et al. Effect of pamidronate administration on bone in patients with acute spinal cord injury. J Rehabil Res Dev. 2005; 42(3):305-13. Epub 2005/09/28. PubMed PMID: 16187243.
Bauman W A, Cardozo C. Immobilization Osteoporosis. In: Marcus R, Nelson D, Rosen C J, editors. Osteoporosis Fourth Edition: Academic Press; 2013.
Bauman, W. A., M. F. La Fountaine, and A. M. Spungen, Age-related prevalence of low testosterone in men with spinal cord injury. J Spinal Cord Med, 2014. 37(1): p. 32-9
Bauman W A, Cirnigliaro C M, La Fountaine M F, Martinez L, Kirshblum S C, Spungen A M. Zoledronic acid administration failed to prevent bone loss at the knee in persons with acute spinal cord injury: an observational cohort study. J Bone Miner Metab. 2015; 33(4):410-21. Epub 2014/08/28. doi: 10.1007/s00774-014-0602-x. PubMed PMID: 25158630.
Bauman W A, Cardozo C P. Osteoporosis in individuals with spinal cord injury. P M R. 2015; 7(2):188-201; quiz Epub 2014/08/31. doi: 51934-1482(14)01358-6 [pii]10.1016/j.pmrj.2014.08.948. PubMed PMID: 25171878.
Bone H G. Ten Years of Denosumab Treatment in Postmenopausal Women With Osteoporosis: Results From the FREEDOM Extension Trial. The 2015 ASBMR Annual Meeting. 2015; LB-1157.
Bramlett H M, Dietrich W D, Marcillo A, Mawhinney L J, Furones-Alonso O, Bregy A, et al. Effects of low intensity vibration on bone and muscle in rats with spinal cord injury. Osteoporos Int. 2014; 25(9):2209-19. Epub 2014/05/28. doi: 10.1007/s00198-014-2748-8. PubMed PMID: 24861907.
Brouwers J E, Lambers F M, van Rietbergen B, Ito K, Huiskes R. Comparison of bone loss induced by ovariectomy and neurectomy in rats analyzed by in vivo micro-C T. J Orthop Res. 2009; 27(11):1521-7. Epub 2009/05/14. doi: 10.1002/jor.20913. PubMed PMID: 19437511.
Bubbear J S, Gall A, Middleton F R, Ferguson-Pell M, Swaminathan R, Keen R W. Early treatment with zoledronic acid prevents bone loss at the hip following acute spinal cord injury. Osteoporos Int. 2011; 22(1):271-9. Epub 2010/04/02. doi: 10.1007/s00198-010-1221-6. PubMed PMID: 20358358.
Cardozo, C. P., et al., Nandrolone slows hindlimb bone loss in a rat model of bone loss due to denervation. Ann N Y Acad Sci, 2010. 1192: p. 303-6.
Chandrasekhar, K. S., et al., Blood vessel wall-derived endothelial colony-forming cells enhance fracture repair and bone regeneration. Calcif Tissue Int, 2011. 89(5): p. 347-57.
Cirnigliaro C M, La Fountaine M F, Parrott J S, Kirshblum S C, McKenna C, Sauer S J, et al. Administration of Denosumab Preserves Bone Mineral Density at the Knee in Persons With Subacute Spinal Cord Injury: Findings From a Randomized Clinical Trial. JBMR Plus. 2020; 4(8):e10375. doi: 10.1002/jbm4.10375. PubMed PMID: 33134767; PubMed Central PMCID: PMCPMC7587457.
Cosman F, Crittenden D B, Adachi J D, Binkley N, Czerwinski E, Ferrari S, et al. Romosozumab Treatment in Postmenopausal Women with Osteoporosis. N Engl J Med. 2016; 375(16):1532-43. doi: 10.1056/NEJMoa1607948. PubMed PMID: 27641143.
Cosman F, Nieves J, Zion M, Woelfert L, Luckey M, Lindsay R. Daily and cyclic parathyroid hormone in women receiving alendronate. N Engl J Med. 2005; 353(6):566-75. Epub 2005/08/12. doi: 353/6/566 [pii]10.1056/NEJMoa050157. PubMed PMID: 16093465.
Cragg J J, Noonan V K, Krassioukov A, Borisoff J. Cardiovascular disease and spinal cord injury: results from a national population health survey. Neurology. 2013; 81(8):723-8. doi: 10.1212/WNL.0b013e3182a1aa68. PubMed PMID: 23884034; PubMed Central PMCID: PMCPMC3776463.
Cummings S R, McCulloch C. Explanations for the difference in rates of cardiovascular events in a trial of alendronate and romosozumab. Osteoporos Int. 2020; 31(6):1019-21. doi: 10.1007/s00198-020-05379-z. PubMed PMID: 32246168.
Edwards W B, Simonian N, Haider I T, Anschel A S, Chen D, Gordon K E, et al. Effects of Teriparatide and Vibration on Bone Mass and Bone Strength in People with Bone Loss and Spinal Cord Injury: A Randomized, Controlled Trial. J Bone Miner Res. 2018; 33(10):1729-40. doi: 10.1002/jbmr.3525. PubMed PMID: 29905973.
Fehlings, M. G., D. W. Cadotte, and L. N. Fehlings, A series of systematic reviews on the treatment of acute spinal cord injury: a foundation for best medical practice. J Neurotrauma, 2011. 28(8): p. 1329-33.
Francis, R. M., The effects of testosterone on osteoporosis in men. Clin Endocrinol (Oxf), 1999. 50(4): p. 411-4.
Freemantle N, Cooper C, Diez-Perez A, Gitlin M, Radcliffe H, Shepherd S, et al. Results of indirect and mixed treatment comparison of fracture efficacy for osteoporosis treatments: a meta-analysis. Osteoporos Int. 2012. Epub 2012/07/27. doi: 10.1007/s00198-012-2068-9. PubMed PMID: 22832638.
Garland D E, Adkins R H, Kushwaha V, Stewart C. Risk factors for osteoporosis at the knee in the spinal cord injury population. J Spinal Cord Med. 2004; 27(3):202-6. PubMed PMID: 15478520.
Gifre L, Vidal J, Carrasco J L, Muxi A, Portell E, Monegal A, et al. Denosumab increases sublesional bone mass in osteoporotic individuals with recent spinal cord injury. Osteoporos Int. 2016; 27(1):405-10. doi: 10.1007/s00198-015-3333-5. PubMed PMID: 26423406.
Gilchrist N L, Frampton C M, Acland R H, Nicholls M G, March R L, Maguire P, et al. Alendronate prevents bone loss in patients with acute spinal cord injury: a randomized, double-blind, placebo-controlled study. J Clin Endocrinol Metab. 2007; 92(4):1385-90. Epub 2007/01/18. doi: jc.2006-2013 [pii] 10.1210/jc.2006-2013. PubMed PMID: 17227802.
Gordon K E, Wald M J, Schnitzer T J. Effect of parathyroid hormone combined with gait training on bone density and bone architecture in people with chronic spinal cord injury. PM R. 2013; 5(8):663-71. Epub 2013/04/06. doi: 51934-1482(13)00161-5 [pii] 10.1016/j.pmrj.2013.03.032. PubMed PMID: 23558091.
Hiruma Y; Tsuda E; Maeda N; Okada A; Kabasawa N; Miyamoto M; Hattori H; Fukuda C. Impaired osteoclast differentiation and function and mild osteopetrosis development in Siglec-15-deficient mice. Bone, 2013.
Humphrey M B, Nakamura M C. A Comprehensive Review of Immunoreceptor Regulation of Osteoclasts. Clin Rev Allergy Immunol. 2016; 51(1):48-58. doi: 10.1007/s12016-015-8521-8. PubMed PMID: 26573914; PubMed Central PMCID: PMCPMC4867136.
Ishida-Kitagawa N, Tanaka K, Bao X, Kimura T, Miura T, Kitaoka Y, et al. Siglec-15 protein regulates formation of functional osteoclasts in concert with DNAX-activating protein of 12 kDa (DAP12). J Biol Chem. 2012; 287(21):17493-502. doi: 10.1074/jbc.M111.324194. PubMed PMID: 22451653; PubMed Central PMCID: PMCPMC3366812.
Iwamoto J, Matsumoto H, Takeda T. Efficacy of risedronate against hip fracture in patients with neurological diseases: a meta-analysis of randomized controlled trials. Curr Med Res Opin. 2008; 24(5):1379-84. doi: 10.1185/030079908X297321. PubMed PMID: 18384711.
Jensen, P. R., et al. Bisphosphonates impair the onset of bone formation at remodeling sites. Bone. 202; 145:115850-60.
Jiang S D, Jiang L S, Dai L Y. Mechanisms of osteoporosis in spinal cord injury. Clin Endocrinol (Oxf). 2006; 65(5):555-65. Epub 2006/10/24. doi: CEN2683 [pii]10.1111/j.1365-2265.2006.02683.x. PubMed PMID: 17054455.
Jiang S D, Jiang L S, Dai L Y. Effects of spinal cord injury on osteoblastogenesis, osteoclastogenesis and gene expression profiling in osteoblasts in young rats. Osteoporos Int. 2007; 18(3):339-49. Epub 2006/10/13. doi: 10.1007/s00198-006-0229-4. PubMed PMID: 17036173.
Kameda Y, Takahata M, Mikuni S, Shimizu T, Hamano H, Angata T, et al. Siglec-15 is a potential therapeutic target for postmenopausal osteoporosis. Bone. 2015; 71:217-26. doi: 10.1016/j.bone.2014.10.027. PubMed PMID: 25460183.
Kendler D L, Roux C, Benhamou C L, Brown J P, Lillestol M, Siddhanti S, et al. Effects of denosumab on bone mineral density and bone turnover in postmenopausal women transitioning from alendronate therapy. J Bone Miner Res. 2010; 25(1):72-81. Epub 2009/07/15. doi: 10.1359/jbmr.090716. PubMed PMID: 19594293.
Khosla S, Hofbauer L C. Osteoporosis treatment: recent developments and ongoing challenges. Lancet Diabetes Endocrinol. 2017; 5(11):898-907. doi: 10.1016/52213-8587(17)30188-2. PubMed PMID: 28689769; PubMed Central PMCID: PMCPMC5798872.
Kondo H, Nifuji A, Takeda S, Ezura Y, Rittling S R, Denhardt D T, et al. Unloading induces osteoblastic cell suppression and osteoclastic cell activation to lead to bone loss via sympathetic nervous system. J Biol Chem. 2005; 280(34):30192-200. Epub 2005/06/18. doi: M504179200 [pii] 10.1074/jbc.M504179200. PubMed PMID: 15961387.
Kostovski, E., et al., Decreased levels of testosterone and gonadotrophins in men with long-standing tetraplegia. Spinal Cord, 2008. 46(8): p. 559-64.
LeBlanc A D, Schneider V S, Evans H J, Engelbretson D A, Krebs J M. Bone mineral loss and recovery after 17 weeks of bed rest. J Bone Miner Res. 1990; 5(8):843-50. doi: 10.1002/jbmr.5650050807. PubMed PMID: 2239368.
LeBlanc, A. D. et al. Skeletal responses to space flight and the bed rest analog: A review. J Musculoskelet Neuronal Interact, 2007. 7(1): p. 33-47.
Lewiecki E M. New and emerging concepts in the use of denosumab for the treatment of osteoporosis. Ther Adv Musculoskelet Dis. 2018; 10(11):209-23. doi: 10.1177/1759720X18805759. PubMed PMID: 30386439; PubMed Central PMCID: PMCPMC6204627.
M. L P. Data on disability from the National health Interview Survey, 1983-85. An InfoUse Report Washington, DC. National Institute on Disability and Rehabilitation Research. 1988.
McClung M R, Grauer A. Romosozumab in postmenopausal women with osteopenia. N Engl J Med. 2014; 370(17):1664-5. doi: 10.1056/NEJMc1402396. PubMed PMID: 24758630.
McDonald M M, Khoo W H, Ng P Y, Xiao Y, Zamerli J, Thatcher P, et al. Osteoclasts recycle via osteomorphs during RANKL-stimulated bone resorption. Cell. 2021; 184(7):1940. doi: 10.1016/j.cell.2021.03.010. PubMed PMID: 33798441; PubMed Central PMCID: PMCPMC8024244.
Minaire P, Neunier P, Edouard C, Bernard J, Courpron P, Bourret J. Quantitative histological data on disuse osteoporosis: comparison with biological data. Calcif Tissue Res. 1974; 17(1):57-73. Epub 1974/01/01. PubMed PMID: 4451877.
Morri, M., Ambrosi, E., Chiari, P. et al. One-year mortality after hip fracture surgery and prognostic factors: a prospective cohort study. Sci Rep 9, 18718 (2019).
Morse L R, Battaglino R A, Stolzmann K L, Hallett L D, Waddimba A, Gagnon D, et al. Osteoporotic fractures and hospitalization risk in chronic spinal cord injury. Osteoporos Int. 2009; 20(3):385-92. Epub 2008/06/27. doi: 10.1007/s00198-008-0671-6. PubMed PMID: 18581033; PubMed Central PMCID: PMC2640446.
Morse L R, Giangregorio L, Battaglino R A, Holland R, Craven B C, Stolzmann K L, et al. VA-based survey of osteoporosis management in spinal cord injury. PM R. 2009; 1(3):240-4. doi: 10.1016/j.pmrj.2008.10.008. PubMed PMID: 19627901; PubMed Central PMCID: PMCPMC2791704.
Murad M H, Drake M T, Mullan R J, Mauck K F, Stuart L M, Lane M A, et al. Clinical review. Comparative effectiveness of drug treatments to prevent fragility fractures: a systematic review and network meta-analysis. J Clin Endocrinol Metab. 2012; 97(6):1871-80. Epub 2012/04/03. doi: 10.1210/jc.2011-3060. PubMed PMID: 22466336.
National Spinal Cord Injury Statistical Center. Spinal cord injury facts and figures at a glance. 2018.
Notelovitz, M., Androgen effects on bone and muscle. Fertil Steril, 2002. 77 Suppl 4: p. S34-41.
Park-Wyllie L Y, Mamdani M M, Juurlink D N, Hawker G A, Gunraj N, Austin P C, et al. Bisphosphonate use and the risk of subtrochanteric or femoral shaft fractures in older women. JAMA. 2011; 305(8):783-9. doi: 10.1001/jama.2011.190. PubMed PMID: 21343577.
Pederson, L., et al., Androgens regulate bone resorption activity of isolated osteoclasts in vitro. Proc Natl Acad Sci USA, 1999. 96(2): p. 505-10.
Peng Y, Langermann, S., Liu, L., Zhao, W., Hu, Y., Guo, X. E, Chen, L., Bauman, W. A., Qin, W. Efficacy of a Novel Antiresorptive Agent-anti-Siglec-15 Antibody to Prevent Bone Loss after Acute Spinal Cord Injury in Rats. ASBMR 2021 Annual Meeting. 2021; VPL-415 (Virtual Plenary Poster Presentations).
Qin W, Bauman W A, Cardozo C. Bone and muscle loss after spinal cord injury: organ interactions. Ann N Y Acad Sci. 2010; 1211:66-84. Epub 2010/11/11. doi: 10.1111/j.1749-6632.2010.05806.x. PubMed PMID: 21062296.
Qin W, Bauman W A, Cardozo C P. Evolving concepts in neurogenic osteoporosis. Curr Osteoporos Rep. 2010; 8(4):212-8. Epub 2010/09/08. doi: 10.1007/s11914-010-0029-9. PubMed PMID: 20820963.
Qin W, Sun L, Cao J, Peng Y, Collier L, Wu Y, et al. The Central Nervous System (CNS)-independent Anti-bone-resorptive Activity of Muscle Contraction and the Underlying Molecular and Cellular Signatures. J Biol Chem. 2013; 288(19):13511-21. Epub 2013/03/27. doi: M113.454892 [pii]10.1074/jbc.M113.454892. PubMed PMID: 23530032; PubMed Central PMCID: PMC3650388.
Qin W, Li X, Peng Y, Harlow L M, Ren Y, Wu Y, et al. Sclerostin antibody preserves the morphology and structure of osteocytes and blocks the severe skeletal deterioration after motor-complete spinal cord injury in rats. J Bone Miner Res. 2015; 30(11):1994-2004. doi: 10.1002/jbmr.2549. PubMed PMID: 25974843.
Qin W, Zhao W, Li X, Peng Y, Harlow L M, Li J, et al. Mice with sclerostin gene deletion are resistant to the severe sublesional bone loss induced by spinal cord injury. Osteoporos Int. 2016; 27(12):3627-36. doi: 10.1007/s00198-016-3700-x. PubMed PMID: 27436301.
Recker R, Lappe J, Davies K, Heaney R. Characterization of perimenopausal bone loss: a prospective study. J Bone Miner Res. 2000; 15(10):1965-73. doi: 10.1359/jbmr.2000.15.10.1965. PubMed PMID: 11028449.
Rolvien, T. and Amling, M. Disuse Osteoporosis: Clinical and Mechanistic Insights. Calif Tissue Int. 2022; 110(5): p. 592-604.
Reid I R, Miller P D, Brown J P, Kendler D L, Fahrleitner-Pammer A, Valter I, et al. Effects of denosumab on bone histomorphometry: the FREEDOM and STAND studies. J Bone Miner Res. 2010; 25(10):2256-65. Epub 2010/06/10. doi: 10.1002/jbmr.149. PubMed PMID: 20533525.
Saag K G, Petersen J, Brandi M L, Karaplis A C, Lorentzon M, Thomas T, et al. Romosozumab or Alendronate for Fracture Prevention in Women with Osteoporosis. N Engl J Med. 2017; 377(15):1417-27. doi: 10.1056/NEJMoa1708322. PubMed PMID: 28892457.
Saita, Y., et al., Combinatory effects of androgen receptor deficiency and hind limb unloading on bone. Horm Metab Res, 2009. 41(11): p. 822-8.
Sato D, Takahata M, Ota M, Fukuda C, Hasegawa T, Yamamoto T, et al. Siglec-15-targeting therapy protects against glucocorticoid-induced osteoporosis of growing skeleton in juvenile rats. Bone. 2020; 135:115331. doi: 10.1016/j.bone.2020.115331. PubMed PMID: 32217159.
Schopp, L. H., et al., Testosterone levels among men with spinal cord injury admitted to inpatient rehabilitation. Am J Phys Med Rehabil, 2006. 85(8): p. 678-84; quiz 685-7.
Shen V, Liang X G, Birchman R, Wu D D, Healy D, Lindsay R, et al. Short-term immobilization-induced cancellous bone loss is limited to regions undergoing high turnover and/or modeling in mature rats. Bone. 1997; 21(1):71-8. Epub 1997/07/01. PubMed PMID: 9213010.
Stuible M, Moraitis A, Fortin A, Saragosa S, Kalbakji A, Filion M, et al. Mechanism and function of monoclonal antibodies targeting siglec-15 for therapeutic inhibition of osteoclastic bone resorption. J Biol Chem. 2014; 289(10):6498-512. doi: 10.1074/jbc.M113.494542. PubMed PMID: 24446437; PubMed Central PMCID: PMCPMC3945315.
Sun L, Pan J, Peng Y, Wu Y, Li J, Liu X, et al. Anabolic steroids reduce spinal cord injury-related bone loss in rats associated with increased Wnt signaling. J Spinal Cord Med. 2013; 36(6):616-22. Epub 2013/10/05. doi: jscm-d-12-00035 [pii] 10.1179/2045772312Y.0000000020. PubMed PMID: 24090150; PubMed Central PMCID: PMC3831322.
Szollar S M, Martin E M, Sartoris D J, Parthemore J G, Deftos L J. Bone mineral density and indexes of bone metabolism in spinal cord injury. Am J Phys Med Rehabil. 1998; 77(1):28-35. PubMed PMID: 9482376.
Trinh, A., et al. Fractures in spina bifida from childhood to young adulthood. Osteoporos. Int., 2017. 28(1): p. 399-406.
Tsitouras, P. D., et al., Serum testosterone and growth hormone/insulin-like growth factor-I in adults with spinal cord injury. Horm Metab Res, 1995. 27(6): p. 287-92.
Tsukazaki H, Kikuta J, Ao T, Morimoto A, Fukuda C, Tsuda E, et al. Anti-Siglec-15 antibody suppresses bone resorption by inhibiting osteoclast multinucleation without attenuating bone formation. Bone. 2021; 152:116095. doi: 10.1016/j.bone.2021.116095. PubMed PMID: 34216837.
US Department of Veteran Affairs. VA and Spinal Cord Injury: Fact Sheet. 2022; https://www.research.va.gov/topics/sci.cfm.
Vestergaard P, Krogh K, Rejnmark L, Mosekilde L. Fracture rates and risk factors for fractures in patients with spinal cord injury. Spinal Cord. 1998; 36(11):790-6. Epub 1998/12/16. PubMed PMID: 9848488.
Vico, L., et al. Cortical and Trabecular Bone Microstructure Did Not Recover at Weight-Bearing Skeletal Sites and Progressively Deteriorated at Non-Weight-Bearing Sites During the Year Following International Space Station Missions. J Bone Miner Res., 2017. 32(10): p. 2010-2021.
Vico L, Collet P, Guignandon A, Lafage-Proust M H, Thomas T, Rehaillia M, et al. Effects of long-term microgravity exposure on cancellous and cortical weight-bearing bones of cosmonauts. Lancet. 2000; 355(9215):1607-11. PubMed PMID: 10821365.
Warden S J, Bennell K L, Matthews B, Brown D J, McMeeken J M, Wark J D. Quantitative ultrasound assessment of acute bone loss following spinal cord injury: a longitudinal pilot study. Osteoporos Int. 2002; 13(7):586-92. doi: 10.1007/s001980200077. PubMed PMID: 12111020.
Won, J. H. and Jung, S. H. Bone Mineral Density in Adults with Cerebral Palsy. Front. Neurol., 2021(12).
Xie, H., et al. PDGF-BB secreted by preosteoclasts induces angiogenesis during coupling with osteogenesis. Nat. Med. 20, 1270-1278 (2014).
Yarrow J F, Kok H J, Phillips E G, Conover C F, Lee J, Bassett T E, et al. Locomotor training with adjuvant testosterone preserves cancellous bone and promotes muscle plasticity in male rats after severe spinal cord injury. J Neurosci Res. 2020; 98(5):843-68. doi:
Zaidi, M., Skeletal remodeling in health and disease. Nat Med, 2007. 13(7): p. 791-801.
Zehnder Y, Luthi M, Michel D, Knecht H, Perrelet R, Neto I, et al. Long-term changes in bone metabolism, bone mineral density, quantitative ultrasound parameters, and fracture incidence after spinal cord injury: a cross-sectional observational study in 100 paraplegic men. Osteoporos Int. 2004; 15(3):180-9. Epub 2004/01/15. doi: 10.1007/s00198-003-1529-6. PubMed PMID: 14722626.
Zhang G, Guo B, Wu H, Tang T, Zhang B T, Zheng L, et al. A delivery system targeting bone formation surfaces to facilitate RNAi-based anabolic therapy. Nat Med. 2012; 18(2):307-14. Epub 2012/01/31. doi: nm.2617 [pii]10.1038/nm.2617. PubMed PMID: 22286306.
Zhao W, Li X, Peng Y, Qin Y, Pan J, Li J, et al. Sclerostin Antibody Reverses the Severe Sublesional Bone Loss in Rats After Chronic Spinal Cord Injury. Calcif Tissue Int. 2018; 103(4):443-54. doi: 10.1007/s00223-018-0439-8. PubMed PMID: 29931461.
Zhen G, Dan Y, Wang R, Dou C, Guo Q, Zarr M, et al. An antibody against Siglec-15 promotes bone formation and fracture healing by increasing TRAP(+) mononuclear cells and PDGF-BB secretion. Bone Res. 2021; 9(1):47. doi: 10.1038/s41413-021-00161-1. PubMed PMID: 34719673; PubMed Central PMCID: PMCPMC8558327.
Zhu Y, Yao S, Chen L. Cell surface signaling molecules in the control of immune responses: a tide model. Immunity. 2011; 34(4):466-78. doi: 10.1016/j.immuni.2011.04.008. PubMed PMID: 21511182; PubMed Central PMCID: PMCPMC3176719.10.1002/jnr.24564. PubMed PMID: 31797423; PubMed Central PMCID: PMCPMC7072003.

What is claimed is:

1. A method of treating bone loss in a subject in need thereof, comprising administering an effective amount of a composition comprising an immunomodulatory agent that modulates Siglec-15 expression, ligand binding, crosslinking, Siglec-15 mediated signaling or a combination thereof selected from a group consisting of
(i) a soluble Siglec-15 polypeptide or fusion protein,
(ii) a function blocking or function activating anti-Siglec 15 antibody,
(iii) a monoclonal antibody or antigen-binding fragment thereof that depletes Siglec-15 positive cells, and
(iv) combinations thereof;
to modulate Siglec-15 mediated signaling in subject in need thereof.

2. The method of claim 1, wherein the monoclonal antibody or antigen-binding fragment thereof comprises a light chain variable region (LCVR) having an amino acids sequence of at least 98%, 99% or more sequence identity to SEQ ID NO: 3, 7, 8, 9, 10, 23, 27, 28, 29, 31, 33, 35, or a variants thereof, and wherein the monoclonal antibody or antigen-binding fragment thereof exhibits binding to Siglec-15.

3. The method of claim 1, wherein the monoclonal antibody or antigen-binding fragment thereof comprises a heavy chain variable region (HCVR) having an amino acid sequence of least 98%, 99% or more sequence identity to SEQ ID NO: 11, 15, 16, 17, 19, 21, 37, 41, 42, 45, 46 or variants thereof, and wherein the monoclonal antibody or antigen-binding fragment thereof exhibits binding to Siglec-15.

4. The method of claim 1, wherein the monoclonal antibody or antigen-binding fragment thereof is a humanized anti-Siglec 15 antibody having a variable heavy chain amino acid sequence with 98%, 99% or more sequence identity to SEQ ID NOs: 16, 17, 19, 21, 42, 45, 46, or variants thereof; and a variable light chain amino acid sequence with 98%, 99% or more sequence identity to SEQ ID NOs: 8, 9, 10, 28, 29, 31, 33, 35, or variants thereof.

5. The method of claim 4, wherein the monoclonal antibody or antigen-binding fragment thereof comprises one or more light chain CDR (LCDR) sequences having an amino acid sequences with 98%, 99% or 100% sequence identity to SEQ ID NO:4, 5, 6, 24, 25, 26, 30, 32, 34, 36 or combinations thereof; and one or more heavy chain CDR (HCDR) sequences having an amino acid sequences with 98%, 99% or 100% sequence identity to SEQ ID NO: 12, 13, 14, 18, 20, 22, 38, 39, 40, 43, 44, 47, or combinations thereof.

6. The method of claim 4, wherein the monoclonal antibody or antigen-binding fragment thereof comprises:
a) a HCDR1 domain having 98%, 99% or more sequence identity to sequences selected from the group consisting of SEQ ID NOs: 12, 38, 43, and 47;
b) a HCDR2 domain having 98%, 99% or more sequence identity to sequences selected from the group consisting of SEQ ID NO: 13, 18, 20, 22, 39, and 44;
c) a HCDR3 domain having 98%, 99% or more sequence identity to sequences selected from the group consisting of SEQ ID NO: 14 and 40;
d) a LCDR1 domain having 98%, 99% or more sequence identity to sequences selected from the group consisting of SEQ ID NOs: 4, 24, 30, 32 and 34;
e) a LCDR2 domain having 98%, 99% or more sequence identity to sequences selected from the group consisting SEQ ID NOs: 5, 25, and 36;
f) a LCDR3 domain having 98%, 99% or more sequence identity to sequences selected from the group consisting SEQ ID NO: 6, and 26; or combinations thereof.

7. The method of claim 1, wherein the monoclonal antibody or antigen-binding fragment thereof comprises a HCVR sequence having 98%, 99% or more sequence identity to:
a) SEQ ID NO: 11 having (a) an HCDR1 domain having 98%, 99% or more sequence identity to SEQ ID NOs: 12; (b) a HCDR2 domain having 98%, 99% or more sequence identity to SEQ ID NO: 13; (c) an HCDR3 domain having 98%, 99% or more sequence identity to SEQ ID NO: 14;
b) SEQ ID NO: 16 having (a) an HCDR1 domain having 98%, 99% or more sequence identity to SEQ ID NOs: 12; (b) a HCDR2 domain having 98%, 99% or more sequence identity to SEQ ID NO: 13; (c) an HCDR3 domain having 98%, 99% or more sequence identity to SEQ ID NO: 14;
c) SEQ ID NO: 17 having (a) an HCDR1 domain having 98%, 99% or more sequence identity to SEQ ID NOs: 12; (b) a HCDR2 domain having 98%, 99% or more sequence identity to SEQ ID NO: 18; (c) an HCDR3 domain having 98%, 99% or more sequence identity to SEQ ID NO: 14;
d) SEQ ID NO: 19 having (a) an HCDR1 domain having 98%, 99% or more sequence identity to SEQ ID NOs: 12; (b) a HCDR2 domain having 98%, 99% or more sequence identity to SEQ ID NO: 20; (c) an HCDR3 domain having 98%, 99% or more sequence identity to SEQ ID NO: 14;
e) SEQ ID NO: 21 having (a) an HCDR1 domain having 98%, 99% or more sequence identity to SEQ ID NOs: 12; (b) a HCDR2 domain having 98%, 99% or more sequence identity to SEQ ID NO: 22; (c) an HCDR3 domain having 98%, 99% or more sequence identity to SEQ ID NO: 14;
f) SEQ ID NO: 37 having (a) an HCDR1 domain having 98%, 99% or more sequence identity to SEQ ID NOs: 38; (b) a HCDR2 domain having 98%, 99% or more sequence identity to SEQ ID NO: 39; (c) an HCDR3 domain having 98%, 99% or more sequence identity to SEQ ID NO: 40;
g) SEQ ID NO: 42 having (a) an HCDR1 domain having 98%, 99% or more sequence identity to SEQ ID NOs: 43; (b) a HCDR2 domain having 98%, 99% or more sequence identity to SEQ ID NO: 44; (c) an HCDR3 domain having 98%, 99% or more sequence identity to SEQ ID NO: 40;
h) SEQ ID NO: 45 having (a) an HCDR1 domain having 98%, 99% or more sequence identity to SEQ ID NOs: 43; (b) a HCDR2 domain having 98%, 99% or more sequence identity to SEQ ID NO: 44; (c) an HCDR3 domain having 98%, 99% or more sequence identity to SEQ ID NO: 40; or
i) SEQ ID NO: 46 having (a) an HCDR1 domain having 98%, 99% or more sequence identity to SEQ ID NOs: 47; (b) a HCDR2 domain having 98%, 99% or more sequence identity to SEQ ID NO: 44; (c) an HCDR3 domain having 98%, 99% or more sequence identity to SEQ ID NO: 40.

8. The method of claim 1, wherein the monoclonal antibody or antigen-binding fragment thereof comprises a LCVR sequence having 98%, 99% or more sequence identity to:
a) SEQ ID NO: 3 having (a) an LCDR1 domain having 98%, 99% or more sequence identity to SEQ ID NOs: 4; (b) a LCDR2 domain having 98%, 99% or more sequence identity to of SEQ ID NO: 5; (c) an LCDR3 domain having 98%, 99% or more sequence identity to SEQ ID NO: 6;
b) SEQ ID NO: 8 having (a) an LCDR1 domain having 95%, 96%, 97%, 98%, 99% or more sequence identity to SEQ ID NOs: 4; (b) a LCDR2 domain having 95%, 96%, 97%, 98%, 99% or more sequence identity to SEQ ID NO: 5; (c) an LCDR3 domain having 95%, 96%, 97%, 98%, 99% or more sequence identity to SEQ ID NO: 6;
c) SEQ ID NO: 9 having (a) an LCDR1 domain having 95%, 96%, 97%, 98%, 99% or more sequence identity to SEQ ID NOs: 4; (b) a LCDR2 domain having 95%, 96%, 97%, 98%, 99% or more sequence identity to SEQ ID NO: 5; (c) an LCDR3 domain having 95%, 96%, 97%, 98%, 99% or more sequence identity to SEQ ID NO: 6;
d) SEQ ID NO: 10 having (a) an LCDR1 domain having 95%, 96%, 97%, 98%, 99% or more sequence identity to SEQ ID NOs: 4; (b) a LCDR2 domain having 95%, 96%, 97%, 98%, 99% or more sequence identity to SEQ ID NO: 5; (c) an LCDR3 domain having 95%, 96%, 97%, 98%, 99% or more sequence identity to SEQ ID NO: 6;
e) SEQ ID NO: 23 having (a) an LCDR1 domain having 95%, 96%, 97%, 98%, 99% or more sequence identity to SEQ ID NOs: 24; (b) a LCDR2 domain having 95%, 96%, 97%, 98%, 99% or more sequence identity to SEQ ID NO: 25; (c) an LCDR3 domain having 95%, 96%, 97%, 98%, 99% or more sequence identity to SEQ ID NO: 26;
f) SEQ ID NO: 28 having (a) an LCDR1 domain having 95%, 96%, 97%, 98%, 99% or more sequence identity to SEQ ID NOs: 24; (b) a LCDR2 domain having 95%, 96%, 97%, 98%, 99% or more sequence identity to SEQ ID NO: 25; (c) an LCDR3 domain having 95%, 96%, 97%, 98%, 99% or more sequence identity to SEQ ID NO: 26;
g) SEQ ID NO: 29 having (a) an LCDR1 domain having 95%, 96%, 97%, 98%, 99% or more sequence identity to SEQ ID NOs: 30; (b) a LCDR2 domain having 95%, 96%, 97%, 98%, 99% or more sequence identity to SEQ ID NO: 25; (c) an LCDR3 domain having 95%, 96%, 97%, 98%, 99% or more sequence identity to SEQ ID NO: 26;
h) SEQ ID NO: 31 having (a) an LCDR1 domain having 95%, 96%, 97%, 98%, 99% or more sequence identity to SEQ ID NOs: 32; (b) a LCDR2 domain having 95%, 96%, 97%, 98%, 99% or more sequence identity to SEQ ID NO: 25; (c) an LCDR3 domain having 95%, 96%, 97%, 98%, 99% or more sequence identity to SEQ ID NO: 26;
i) SEQ ID NO: 33 having (a) an LCDR1 domain having 95%, 96%, 97%, 98%, 99% or more sequence identity to SEQ ID NOs: 34; (b) a LCDR2 domain having 95%, 96%, 97%, 98%, 99% or more sequence identity to SEQ ID NO: 25; (c) an LCDR3 domain having 95%, 96%, 97%, 98%, 99% or more sequence identity to SEQ ID NO: 26; or
j) SEQ ID NO: 35 having (a) an LCDR1 domain having 95%, 96%, 97%, 98%, 99% or more sequence identity to SEQ ID NOs: 32; (b) a LCDR2 domain having 95%, 96%, 97%, 98%, 99% or more sequence identity to SEQ ID NO: 36; (c) an LCDR3 domain having 95%, 96%, 97%, 98%, 99% or more sequence identity to SEQ ID NO: 26.

9. The method of claim of claim 1, wherein the fusion protein comprises an extracellular domain of Siglec-15 for functional variant thereof linked to an immunoglobulin domain, wherein the fusion protein inhibits, reduces, or blocks Siglec-15 mediated signal transduction.

10. The method of claim of claim 9, wherein the fusion protein comprises the amino acid sequence of any one of SEQ ID NO:1 or 2, or a functional variant thereof linked to an immunoglobulin domain, wherein the fusion modulates Siglec-15 mediated signal transduction.

11. The method of claim of claim 10, wherein the fusion protein has 98%, 99%, or 100% sequence identity to any one of SEQ ID NO: 49, 50, 51, 52, 53, and 54.

12. The method of claim 1, wherein the immunomodulatory agent is administered with an additional therapeutic agent that functions to enhance bone formation or inhibit bone loss.

13. The method of claim 12, wherein the therapeutic agent is selected from a group consisting of bisphosphonates, cytokines, other immunotherapeutics, enzymes, antibiotics, growth factors, growth inhibitors, hormones (including testosterone and parathyroid hormone), hormone antagonists, antibodies and bioactive fragments thereof (including humanized, single chain, and chimeric antibodies), antigen and vaccine formulations (including adjuvants), peptide drugs, anti-inflammatories, molecules that target additional bone metabolic pathways and modulate osteoclastogenesis or osteoblastogenesis.

14. The method of claim 1, wherein the immunomodulatory thereof binds osteoclasts to reduce osteoclast maturation and osteoclastogenesis while inducing osteoblastogenesis.

15. A method of promoting an immune response in a subject having bone loss comprising administering an effective amount of the immunomodulatory agent of claim 1 in an amount effective to inhibit or reduce bone loss, maintain bone formation, or promote rebuilding of the bone for a net anabolic outcome in the subject.

16. The method of claim 15, wherein the subject has disuse osteoporosis due to immobilization, reduction of mechanical loading pressure and activity that is required for homeostatic bone maintenance or remodeling.

17. The method of claim 16, wherein immobilization, reduction of pressure and activity is due to a condition selected from a group including spinal cord injury, hip fracture, cerebral palsy, stroke, paralysis, comas, time spent at zero gravity, medical conditions leading to prolonged bed rest, diseases or syndromes by which a person is dependent on a wheelchair for mobility purposes, including but not limited to Guillain-Barre syndrome, Ehlers-Danlos syndrome, Multiple Sclerosis, Muscular Dystrophy, Amyotrophic Lateral Sclerosis, Spina bifida, Parkinson's disease, polio, or activity-limiting rheumatological diseases.
